SEQUENCE LISTING (51 sequences)

SEQ ID NO: 1 - protein, 5 aa, Artificial Sequence
  Synthetic amino acid sequence
  MISC_FEATURE (1)..(5): Residues 1 to 5 may be repeated one or more times.
GSGGS

SEQ ID NO: 2 - protein, 4 aa, Artificial Sequence
  Synthetic amino acid sequence
  MISC_FEATURE (1)..(4): Residues 1 to 4 may be repeated one or more times.
GGGS

SEQ ID NO: 3 - protein, 5 aa, Artificial Sequence
  Synthetic amino acid sequence
  MISC_FEATURE (1)..(5): Residues 1 to 5 may be repeated one or more times.
GGGGS

SEQ ID NO: 4 - protein, 4 aa, Artificial Sequence
  Synthetic amino acid sequence
GGSG

SEQ ID NO: 5 - protein, 5 aa, Artificial Sequence
  Synthetic amino acid sequence
GGSGG

SEQ ID NO: 6 - protein, 5 aa, Artificial Sequence
  Synthetic amino acid sequence
GSGSG

SEQ ID NO: 7 - protein, 5 aa, Artificial Sequence
  Synthetic amino acid sequence
GSGGG

SEQ ID NO: 8 - protein, 5 aa, Artificial Sequence
  Synthetic amino acid sequence
GGGSG

SEQ ID NO: 9 - protein, 5 aa, Artificial Sequence
  Synthetic amino acid sequence
GSSSG

SEQ ID NO: 10 - protein, 15 aa, Artificial Sequence
  Synthetic amino acid sequence
GGGGSGGGGSGGGGS

SEQ ID NO: 11 - DNA, 45 bp, Artificial Sequence
  Synthetic nucleic acid sequence
ggtggcggtggctcgggcggtggtgggtcgggtggcggcggatct

SEQ ID NO: 12 - protein, 5 aa, Artificial Sequence
  Synthetic amino acid sequence
DKTHT

SEQ ID NO: 13 - protein, 4 aa, Artificial Sequence
  Synthetic amino acid sequence
CPPC

SEQ ID NO: 14 - protein, 15 aa, Artificial Sequence
  Synthetic amino acid sequence
CPEPKSCDTPPPCPR

SEQ ID NO: 15 - protein, 12 aa, Artificial Sequence
  Synthetic amino acid sequence
ELKTPLGDTTHT

SEQ ID NO: 16 - protein, 10 aa, Artificial Sequence
  Synthetic amino acid sequence
KSCDKTHTCP

SEQ ID NO: 17 - protein, 7 aa, Artificial Sequence
  Synthetic amino acid sequence
KCCVDCP

SEQ ID NO: 18 - protein, 7 aa, Artificial Sequence
  Synthetic amino acid sequence
KYGPPCP

SEQ ID NO: 19 - protein, 15 aa, Artificial Sequence
  Synthetic amino acid sequence
EPKSCDKTHTCPPCP

SEQ ID NO: 20 - protein, 12 aa, Artificial Sequence
  Synthetic amino acid sequence
ERKCCVECPPCP

SEQ ID NO: 21 - protein, 17 aa, Artificial Sequence
  ELKTPLGDTTHTCPRCP
ELKTPLGDTTHTCPRCP

SEQ ID NO: 22 - protein, 12 aa, Artificial Sequence
  Synthetic amino acid sequence
SPNMVPHAHHAQ

SEQ ID NO: 23 - protein, 15 aa, Artificial Sequence
  Synthetic amino acid sequence
EPKSCDKTYTCPPCP

SEQ ID NO: 24 - protein, 42 aa, Artificial Sequence
  Synthetic amino acid sequence
KRGRKKLLYIFKQPFMRPVQTTQEEDGCSCRFPEEEEGGCEL

SEQ ID NO: 25 - DNA, 126 bp, Artificial Sequence
  Synthetic nucleic acid sequence
aaacggggcagaaagaaactcctgtatatattcaaacaaccatttatgagaccagtacaaactactcaagaggaagacggctgtagctgccgatttccagaagaagaagaaggaggatgtgaactg

SEQ ID NO: 26 - DNA, 126 bp, Artificial Sequence
  Synthetic nucleic acid sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 27 - protein, 41 aa, Artificial Sequence
  Synthetic amino acid sequence
RSKRSRLLHSDYMNMTPRRPGPTRKHYQPYAPPRDFAAYRS

SEQ ID NO: 28 - DNA, 123 bp, Artificial Sequence
  Synthetic nucleic acid sequence
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 29 - protein, 41 aa, Artificial Sequence
  Synthetic amino acid sequence
RSKRSRLLHSDYMFMTPRRPGPTRKHYQPYAPPRDFAAYRS

SEQ ID NO: 30 - DNA, 123 bp, Artificial Sequence
  Synthetic nucleic acid sequence
aggagtaagaggagcaggctcctgcacagtgactacatgttcatgactccccgccgccccgggcccacccgcaagcattaccagccctatgccccaccacgcgacttcgcagcctatcgctcc

SEQ ID NO: 31 - protein, 35 aa, Artificial Sequence
  Synthetic amino acid sequence
TKKKYSSSVHDPNGEYMFMRAVNTAKKSRLTDVTL

SEQ ID NO: 32 - DNA, 105 bp, Artificial Sequence
  Synthetic nucleic acid sequence
acaaaaaagaagtattcatccagtgtgcacgaccctaacggtgaatacatgttcatgagagcagtgaacacagccaaaaaatccagactcacagatgtgacccta

SEQ ID NO: 33 - DNA, 105 bp, Artificial Sequence
  Synthetic nucleic acid sequence
acaaaaaagaagtattcatccagtgtgcacgaccctaacggtgaatacatgttcatgagagcagtgaacacagccaaaaaatctagactcacagatgtgacccta

SEQ ID NO: 34 - protein, 35 aa, Artificial Sequence
  Synthetic amino acid sequence
TKKKYSSSVHDPNGEYMNMRAVNTAKKSRLTDVTL

SEQ ID NO: 35 - DNA, 105 bp, Artificial Sequence
  Synthetic nucleic acid sequence
acaaaaaagaagtattcatccagtgtgcacgaccctaacggtgaatacatgaacatgagagcagtgaacacagccaaaaaatccagactcacagatgtgacccta

SEQ ID NO: 36 - protein, 117 aa, Artificial Sequence
  Synthetic amino acid sequence
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 37 - DNA, 351 bp, Artificial Sequence
  Synthetic nucleic acid sequence
accaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaat

SEQ ID NO: 38 - protein, 48 aa, Artificial Sequence
  Synthetic amino acid sequence
QRRKYRSNKGESPVEPAEPCRYSCPREEEGSTIPIQEDYRKPEPACSP

SEQ ID NO: 39 - DNA, 144 bp, Artificial Sequence
  Synthetic nucleic acid sequence
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 40 - protein, 42 aa, Artificial Sequence
  Synthetic amino acid sequence
ALYLLRRDQRLPPDAHKPPGGGSFRTPIQEEQADAHSTLAKI

SEQ ID NO: 41 - DNA, 126 bp, Artificial Sequence
  Synthetic nucleic acid sequence
gccctgtacctgctccgcagggaccagaggctgccccccgatgcccacaagccccctgggggaggcagtttcaggacccccatccaagaggagcaggccgacgcccactccaccctggccaagatc

SEQ ID NO: 42 - protein, 112 aa, Artificial Sequence
  Synthetic amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 336 bp, Artificial Sequence
  Synthetic nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 44 - protein, 112 aa, Artificial Sequence
  Synthetic amino acid sequence
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 336 bp, Artificial Sequence
  Synthetic nucleic acid sequence
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 46 - protein, 24 aa, Artificial Sequence
  Synthetic amino acid sequence
IYIWAPLAGTCGVLLLSLVITLYC

SEQ ID NO: 47 - DNA, 72 bp, Artificial Sequence
  Synthetic nucleic acid sequence
atctacatctgggcgcccttggccgggacttgtggggtccttctcctgtcactggttatcaccctttactgc

SEQ ID NO: 48 - protein, 45 aa, Artificial Sequence
  Synthetic amino acid sequence
TTTPAPRPPTPAPTIASQPLSLRPEACRPAAGGAVHTRGLDFACD

SEQ ID NO: 49 - DNA, 135 bp, Artificial Sequence
  Synthetic nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 50 - protein, 27 aa, Artificial Sequence
  Synthetic amino acid sequence
FWVLVVVGGVLACYSLLVTVAFIIFWV

SEQ ID NO: 51 - DNA, 81 bp, Artificial Sequence
  Synthetic nucleic acid sequence
ttttgggtgctggtggtggttggtggagtcctggcttgctatagcttgctagtaacagtggcctttattattttctgggtg

(19) United States
(12) Patent Application Publication
(10) Pub. No.: US 2021/0060070 A1
(43) Pub. Date: Mar. 4, 2021

(54) ADOPTIVE CELL THERAPY AND METHODS OF DOSING THEREOF

(71) Applicant: Tmunity Therapeutics Inc., Philadelphia, PA (US)

(72) Inventor: Christina Coughlin, Philadelphia, PA (US)

(73) Assignee: Tmunity Therapeutics Inc., Philadelphia, PA (US)

(21) Appl. No.: 17/011,636

(22) Filed: Sep. 3, 2020

Related U.S. Application Data
(60) Provisional application No. 62/895,972, filed on Sep. 4, 2019.
(60) Provisional application No. 62/944,884, filed on Dec. 6, 2019.

(51) Int. Cl. A61K 35/17; A61K 31/7076; A61K 31/675; A61P 35/00

(52) U.S. Cl. CPC A61K 35/17; A61P 35/00; A61K 31/675; A61K 31/7076

(57) ABSTRACT

The present disclosure provides methods for the administration of engineered cells, such as T cells, to subjects for adoptive cell therapy. Also provided are compositions and articles of manufacture for use in the methods. The cells express chimeric antigen receptors (CARs) and/or T cell receptors (TCRs), and optionally, other molecules to overcome the immunosuppressive tumor microenvironment. Methods provided herein may employ a fractionated dosing regimen which may further comprise monitoring the development of a toxicity and managing the symptoms thereof.

### CROSS-REFERENCE TO RELATED APPLICATIONS

This application claims the priority benefit under 35 USC § 119 to U.S. provisional Application No. 62/895,972, filed Sep. 4, 2019, and U.S. provisional Application No. 62/944,884, filed Dec. 6, 2019. The entire contents of both applications are incorporated herein by reference in their entirety.

### REFERENCE TO SEQUENCE LISTING SUBMITTED ELECTRONICALLY

The present application contains a Sequence Listing which has been filed electronically in ASCII format and is hereby incorporated by reference in its entirety. The ASCII copy, created Dec. 6, 2019, is named 616485_TMY9-223-2_ST25 and is 18,632 bytes in size.

### BACKGROUND

Novel treatments using T-cells engineered to express chimeric antigen receptors (CAR) have resulted in promising immunotherapies for some types of cancer, primarily hematologic malignancies, with limited data available in solid tumors.

Chimeric antigen receptor (CAR) T cells are effector immune cells that are genetically-modified to recognize a specific tumor-associated antigen and subsequently kill the tumor cell. While success with CAR T therapy has led to approval for use in hematologic malignancy, the effectiveness of CAR T therapy in the treatment of solid tumors, such as breast cancer, remains uncertain. There are several obstacles to CAR T therapy in solid tumors. Foremost, most of the identified and best-studied cell-surface antigens expressed by tumors are also expressed by normal tissue, resulting in non-specific targeting by CAR T cells. Second, solid tumors have a generally immunosuppressive tumor microenvironment, which may inhibit CAR T cell activity once the cells reach the tumor and recognize the antigen. Third, the durability of anti-tumor responses is highly correlated with the persistence of the adoptively-transferred cells and optimal persistence for CAR T cells in solid tumors has yet to match the persistence observed in hematopoietic malignancies.

One of the adverse effects following infusion of CAR T cells is, for example, the onset of immune activation, known as cytokine release syndrome (CRS). CRS is a known on-target toxicity, development of which likely correlates with efficacy. As the efficacy of CAR T therapy improves, the risk of toxicities such as CRS will increase.

Accordingly, a need exists for improved methods in adoptive cell therapy for reducing the risk of toxicities. The present invention provides compositions and methods that address such needs.

### SUMMARY

The present invention is based on novel dosing strategies that address on-target off-tumor toxicities that may result from adoptive cell therapies.

In one aspect, provided is a method of treating a solid tumor in a subject in need thereof, comprising administering to a subject a first dose of cells, wherein the cells comprise a chimeric antigen receptor (CAR) having affinity for a solid tumor antigen, and wherein the first dose comprises about 30% of a total dose of cells; and administering to the subject a consecutive dose of cells comprising the CAR, wherein the consecutive dose comprises about 70% of the total dose of cells, and wherein the consecutive dose is administered at least about five days after the administration of the first dose.

In certain exemplary embodiments, the first dose comprises from about 3×10⁷ cells to about 6×10⁷ cells, and the second dose comprises from about 7×10⁷ cells to about 1.4×10⁸ cells. In certain exemplary embodiments, the first dose comprises from about 1.5×10⁸ cells to about 1.8×10⁸ cells, and the second dose comprises from about 3.5×10⁸ cells to about 4.2×10⁸ cells.

In certain exemplary embodiments, the method further comprises administering to the subject a lymphodepleting chemotherapy. In certain exemplary embodiments, the lymphodepleting chemotherapy comprises a therapeutically effective amount of cyclophosphamide and/or fludarabine. In certain exemplary embodiments, the lymphodepleting chemotherapy comprises a therapeutically effective amount of cyclophosphamide and fludarabine. In certain exemplary embodiments, the therapeutically effective amount of cyclophosphamide is 300 mg/m²/day. In certain exemplary embodiments, the therapeutically effective amount of fludarabine is 30 mg/m²/day.

In certain exemplary embodiments, the lymphodepleting chemotherapy is administered to the subject prior to administering the first dose of cells. In certain exemplary embodiments, the lymphodepleting chemotherapy is administered to the subject four to six days prior to administering the first dose of cells. In certain exemplary embodiments, the lymphodepleting chemotherapy is administered to the subject consecutively for three days.

In certain exemplary embodiments, the solid tumor is a prostate cancer. In certain exemplary embodiments, the prostate cancer is metastatic castrate resistant prostate cancer.

In certain exemplary embodiments, the solid tumor antigen is prostate-specific membrane antigen (PSMA).

In certain exemplary embodiments, the cells comprising the CAR further comprise a dominant negative receptor.

In certain exemplary embodiments, the dominant negative receptor is a truncated variant of a wild-type protein associated with an immunosuppressive signal. In certain exemplary embodiments, the dominant negative receptor is a truncated variant of a TGFβ receptor. In certain exemplary embodiments, the TGFβ receptor is TGFβ receptor type II.

In certain exemplary embodiments, the method further comprises monitoring the development of cytokine release syndrome, immune cell-associated neurologic toxicities, and/or an on-target off-tumor toxicity resulting from the administration of the first dose.

In certain exemplary embodiments, the on-target off-tumor toxicity is parotiditis, and/or a neurologic toxicity associated with the expression of PSMA in a normal tissue. In certain exemplary embodiments, the parotiditis and/or the neurologic toxicity associated with the expression of PSMA in a normal tissue is monitored by a physical examination of the subject, optionally wherein the physical examination comprises assessing the subject for pain or glandular dysfunction.

In certain exemplary embodiments, the consecutive dose is administered at a time when the parotiditis and/or the neurologic toxicity associated with the expression of PSMA in a normal tissue has been treated. In certain exemplary embodiments, the consecutive dose is administered at a time when the parotiditis and/or the neurologic toxicity associated with the expression of PSMA in a normal tissue has subsided.

In certain exemplary embodiments, the normal tissue is a salivary gland and/or the hypothalamus.

In certain exemplary embodiments, the solid tumor is a lung cancer. In certain exemplary embodiments, the lung cancer is non-small cell lung cancer.

In certain exemplary embodiments, the solid tumor is a breast cancer. In certain exemplary embodiments, the breast cancer is triple negative breast cancer.

In certain exemplary embodiments, the solid tumor is a pancreatic cancer. In certain exemplary embodiments, the pancreatic cancer is pancreatic adenocarcinoma.

In certain exemplary embodiments, the solid tumor is an ovarian and fallopian tube cancer.

In certain exemplary embodiments, the solid tumor antigen is mucin-1 (MUC1). In certain exemplary embodiments, the solid tumor antigen is a truncated glycoepitope of MUC1.

In certain exemplary embodiments, the method further comprises monitoring the development of cytokine release syndrome, immune cell-associated neurologic toxicities, and/or an on-target off-tumor toxicity resulting from the administration of the first dose.

In certain exemplary embodiments, the on-target off-tumor toxicity is pancreatitis, renal insufficiency, and/or gastrointestinal inflammation. In certain exemplary embodiments, the pancreatitis, renal insufficiency, and/or gastrointestinal inflammation is monitored by a physical examination or by assessing the blood levels of amylase and/or lipase in the subject after receiving the first dose of cells, compared to the blood levels of amylase and/or lipase of the subject prior to receiving the first dose of cells, optionally wherein the physical examination comprises assessing the subject for abdominal pain.

In certain exemplary embodiments, the consecutive dose is administered at a time when the pancreatitis, renal insufficiency, and/or gastrointestinal inflammation has been treated. In certain exemplary embodiments, the consecutive dose is administered at a time when the pancreatitis, renal insufficiency, and/or gastrointestinal inflammation has subsided.

In another aspect, a method of treating metastatic castrate resistant prostate cancer in a subject in need thereof, comprising: administering to a subject a first dose of cells, wherein the cells comprise a chimeric antigen receptor (CAR) having affinity for prostate-specific membrane antigen (PSMA-CAR), and a truncated variant of TGFβ receptor type II (dnTGFβR2), and wherein the first dose comprises about 30% of a total dose of cells; and administering to the subject a consecutive dose of cells comprising the PSMA-CAR and dnTGFβR2, wherein the consecutive dose comprises about 70% of the total dose of cells, and wherein the consecutive dose is administered at least five days after the administration of the first dose, is provided.

In another aspect, a method of treating metastatic castrate resistant prostate cancer in a subject in need thereof, comprising: administering to a subject a first dose of T cells, wherein the T cells comprise a chimeric antigen receptor (CAR) having affinity for prostate-specific membrane antigen (PSMA-CAR), and a truncated variant of TGFβ receptor type II (dnTGFβR2), and wherein the first dose comprises from about 3×10⁷ cells to about 6×10⁷ cells; and administering to the subject a consecutive dose of T cells comprising the PSMA-CAR and dnTGFβR2, wherein the consecutive dose comprises from about 7×10⁷ cells to about 1.4×10⁸ cells, and wherein the consecutive dose is administered at least five days after the administration of the first dose, is provided

In another aspect, a method of treating metastatic castrate resistant prostate cancer in a subject in need thereof, comprising: administering to a subject a first dose of cells, wherein the cells comprise a chimeric antigen receptor (CAR) having affinity for prostate-specific membrane antigen (PSMA-CAR), and a truncated variant of TGFβ receptor type II (dnTGFβR2), and wherein the first dose comprises from about 1.5×10⁸ cells to about 1.8×10⁸ cells; and administering to the subject a consecutive dose of cells comprising the PSMA-CAR and dnTGFβR2, wherein the consecutive dose comprises from about 3.5×10⁸ cells to about 4.2×10⁸, and wherein the consecutive dose is administered at least five days after the administration of the first dose, is provided.

In certain exemplary embodiments, the method further comprises monitoring the development of cytokine release syndrome, immune cell-associated neurologic toxicities, and/or an on-target off-tumor toxicity resulting from the administration of the first dose.

In certain exemplary embodiments, the on-target off-tumor toxicity is parotiditis, and/or a neurologic toxicity associated with the expression of PSMA in a normal tissue. In certain exemplary embodiments, the parotiditis and/or a neurologic toxicity associated with the expression of PSMA in a normal tissue is monitored by a physical examination of the subject, optionally wherein the physical examination comprises assessing the subject for pain or glandular dysfunction.

In certain exemplary embodiments, the normal tissue is a salivary gland and/or hypothalamus.

In another aspect, a method of treating a solid tumor in a subject in need thereof, comprising: administering to a subject a first dose of cells, wherein the cells comprise a chimeric antigen receptor (CAR) having affinity for a solid tumor antigen, and wherein the first dose comprises about 30% of a total dose of cells; monitoring the development of cytokine release syndrome, immune cell-associated neurologic toxicities, and/or an on-target off-tumor toxicity resulting from the administration of the first dose; and administering to the subject a consecutive dose of cells comprising the CAR, wherein the consecutive dose comprises about 70% of the total dose of cells, and wherein the consecutive dose is administered at least five days after the administration of the first dose, is provided.

In certain exemplary embodiments, the solid tumor is a prostate cancer. In certain exemplary embodiments, the prostate cancer is metastatic castrate-resistant prostate cancer.

In certain exemplary embodiments, the solid tumor antigen is prostate-specific membrane antigen (PSMA).

In certain exemplary embodiments, the cells comprising the CAR further comprise a dominant negative receptor.

In certain exemplary embodiments, the dominant negative receptor is a truncated variant of a wild-type protein associated with an immunosuppressive signal. In certain exemplary embodiments, the dominant negative receptor is a truncated variant of a TGFβ receptor. In certain exemplary embodiments, the TGFβ receptor is TGFβ receptor type II.

In certain exemplary embodiments, the on-target off-tumor toxicity is parotiditis, and/or a neurologic toxicity associated with the expression of PSMA in a normal tissue.

In certain exemplary embodiments, the parotiditis and/or a neurologic toxicity associated with the expression of PSMA in a normal tissue is monitored by a physical examination of the subject, optionally wherein the physical examination comprises assessing the subject for pain or glandular dysfunction.

In certain exemplary embodiments, the normal tissue is a salivary gland and/or hypothalamus.

In certain exemplary embodiments, the solid tumor is a pancreatic cancer. In certain exemplary embodiments, the pancreatic cancer is pancreatic adenocarcinoma.

In certain exemplary embodiments, the solid tumor antigen is mucin-1 (MUC1). In certain exemplary embodiments, the solid tumor antigen is a truncated glycoepitope of MUC1.

In certain exemplary embodiments, the on-target off-tumor toxicity is pancreatitis, renal insufficiency, and/or gastrointestinal inflammation.

In certain exemplary embodiments, the pancreatitis, renal insufficiency, and/or gastrointestinal inflammation is monitored by a physical examination or by assessing the blood levels of amylase and/or lipase in the subject after receiving the first dose of cells, compared to the blood levels of amylase and/or lipase of the subject prior to receiving the first dose of cells, optionally wherein the physical examination comprises assessing the subject for abdominal pain.

In certain exemplary embodiments, the consecutive dose is administered at a time when the on-target off-tumor toxicity has been treated. In certain exemplary embodiments, the consecutive dose is administered at a time when the on-target off-tumor toxicity has subsided.

In another aspect, a method of treating metastatic castrate resistant prostate cancer in a subject in need thereof, comprising: administering to a subject a first dose of cells, wherein the cells comprise a chimeric antigen receptor (CAR) having affinity for prostate-specific membrane antigen (PSMA-CAR), and a truncated variant of TGFβ receptor type II (dnTGFβR2), and wherein the first dose comprises about 30% of a total dose of cells; monitoring the development of parotiditis, and/or a neurologic toxicity associated with the expression of PSMA in a normal tissue resulting from the administration of the first dose; and administering to the subject a consecutive dose of cells comprising the PSMA-CAR and dnTGFβR2, wherein the consecutive dose comprises about 70% of the total dose of cells, and wherein the consecutive dose is administered at least five days after the administration of the first dose, is provided.

In another aspect, a method of treating metastatic castrate resistant prostate cancer in a subject in need thereof, comprising: administering to a subject a first dose of T cells, wherein the T cells comprise a chimeric antigen receptor (CAR) having affinity for prostate-specific membrane antigen (PSMA-CAR), and a truncated variant of TGFβ receptor type II (dnTGFβR2), and wherein the first dose comprises from about 3×10⁷ cells to about 6×10⁷ cells; monitoring the development of parotiditis, and/or a neurologic toxicity associated with the expression of PSMA in a normal tissue resulting from the administration of the first dose; and administering to the subject a consecutive dose of T cells comprising the PSMA-CAR and dnTGFβR2, wherein the consecutive dose comprises from about 7×10⁷ cells to about 1.4×10⁸ cells, and wherein the consecutive dose is administered at least five days after the administration of the first dose, is provided.

In another aspect, a method of treating metastatic castrate resistant prostate cancer in a subject in need thereof, comprising: administering to a subject a first dose of cells, wherein the cells comprise a chimeric antigen receptor (CAR) having affinity for prostate-specific membrane antigen (PSMA-CAR), and a truncated variant of TGFβ receptor type II (dnTGFβR2), and wherein the first dose comprises from about 1.5×10⁸ cells to about 1.8×10⁸ cells; monitoring the development of parotiditis, and/or a neurologic toxicity associated with the expression of PSMA in a normal tissue resulting from the administration of the first dose; and administering to the subject a consecutive dose of cells comprising the PSMA-CAR and dnTGFβR2, wherein the consecutive dose comprises from about 3.5×10⁸ cells to about 4.2×10⁸, and wherein the consecutive dose is administered at least five days after the administration of the first dose, is provided.

In certain exemplary embodiments, the consecutive dose is administered at a time when the parotiditis and/or a neurologic toxicity associated with the expression of PSMA in a normal tissue has been treated. In certain exemplary embodiments, the consecutive dose is administered at a time when the parotiditis and/or a neurologic toxicity associated with the expression of PSMA in a normal tissue has subsided.

In another aspect, a method of treating a cancer in a subject in need thereof, comprising: administering to a subject a first dose of cells, wherein the cells comprise a chimeric antigen receptor (CAR) having affinity for a truncated glycoepitope of mucin-1 (TnMUC1), and wherein the first dose comprises about 30% of a total dose of cells; monitoring the development of pancreatitis, renal insufficiency, and/or gastrointestinal inflammation resulting from the administration of the first dose; and administering to the subject a consecutive dose of cells comprising the TnMUC1-CAR, wherein the consecutive dose comprises about 70% of the total dose of cells, and wherein the consecutive dose is administered at least five days after the administration of the first dose, is provided.

In certain exemplary embodiments, the consecutive dose is administered at a time when the pancreatitis, renal insufficiency, and/or gastrointestinal inflammation has been treated. In certain exemplary embodiments, the consecutive dose is administered at a time when the pancreatitis, renal insufficiency, and/or gastrointestinal inflammation has subsided.

In certain exemplary embodiments, the method further comprises administering to the subject a lymphodepleting chemotherapy. In certain exemplary embodiments, the lymphodepleting chemotherapy comprises a therapeutically effective amount of cyclophosphamide and/or fludarabine. In certain exemplary embodiments, the lymphodepleting chemotherapy comprises a therapeutically effective amount of cyclophosphamide and fludarabine.

In certain exemplary embodiments, the therapeutically effective amount of cyclophosphamide is 300 mg/m²/day.

In certain exemplary embodiments, the therapeutically effective amount of fludarabine is 30 mg/m²/day.

In certain exemplary embodiments, the lymphodepleting chemotherapy is administered to the subject prior to administering the first dose of cells. In certain exemplary embodiments, the lymphodepleting chemotherapy is administered to the subject four to six days prior to administering the first dose of cells. In certain exemplary embodiments, the lymphodepleting chemotherapy is administered to the subject consecutively for three days.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1A and FIG. 1B are schematic representations of the CART-PSMA-TGFβRDN transgene (FIG. 1A) and the structure of the CAR of CART-PSMA-TGFβRDN (FIG. 1B).

FIG. 2 is a schematic depicting the design of the phase I study.

FIG. 3 is a schematic depicting an overview of the CART-PSMA-TGFβRDN manufacturing process.

### DETAILED DESCRIPTION

The present disclosure provides novel methods and compositions for treating a solid tumor in a subject using adoptive cell therapy. In certain embodiments, a method for treating a solid tumor as provided herein comprises administration of a chimeric antigen receptor (CAR) T cell, wherein the CAR has affinity for a solid tumor antigen. In certain embodiments, a method for treating a solid tumor as provided herein comprises a fractionated dosing strategy. Methods of the present disclosure may include monitoring the patient for adoptive cell therapy related side effects that may arise between doses of the fractionated dosing strategy. Such side effects may result generally from adoptive cell therapy, and/or may result from the administration of a CAR T cell directed to a specific solid tumor antigen (e.g., PSMA, MUC1).

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2^{nd} edition).

### A. Chimeric Antigen Receptors

The present invention provides compositions and methods for modified immune cells or precursors thereof, e.g., modified T cells, comprising a chimeric antigen receptor (CAR). Thus, in some embodiments, the immune cell has been genetically modified to express the CAR. CARs of the present invention comprise an antigen binding domain, a transmembrane domain, a hinge domain, and an intracellular signaling domain.

The antigen binding domain may be operably linked to another domain of the CAR, such as the transmembrane domain or the intracellular domain, both described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present invention. A subject CAR of the present invention may also include a spacer domain as described herein. In some embodiments, each of the antigen binding domain, transmembrane domain, and intracellular domain is separated by a linker.

Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g. a tumor associated antigen) on a target cell (e.g. a cancer cell). The target antigen may include any type of protein, or epitope thereof, associated with the target cell. For example, the CAR may comprise affinity to a target antigen on a target cell that indicates a particular status of the target cell.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin.

In an exemplary embodiment, the target cell antigen is a prostate-specific membrane antigen (PSMA). PSMA is a membrane-bound protein expressed on the cell surface and is reported to be highly overexpressed in prostate cancer tissues. PSMA expression is directly correlated with advancing tumor grade and stage, and is believed to confer a selective growth advantage to prostate cancer cells. As such, an exemplary CAR of the present disclosure has affinity for PSMA on a target cell. In an exemplary embodiment, a CAR of the present disclosure having affinity for PSMA on a target cell may comprise a PSMA binding domain. In some embodiments, the PSMA binding domain is a murine PSMA binding domain, e.g., the PSMA binding domain is of murine origin. In some embodiments, the PSMA binding domain is a human PSMA binding domain, e.g., the PSMA binding domain is of human origin.

In some embodiments, the PSMA binding domain is any of the PSMA binding domains disclosed in U.S. Patent Application Ser. No. 62/639,321, or PCT Patent Application No. PCT/US2019/020729, the disclosures of which are incorporated herein by reference in its entirety. Accordingly, a CAR of the present disclosure comprises a PSMA binding domain disclosed in U.S. Patent Application Ser. No. 62/639,321, or PCT Patent Application No. PCT/US2019/020729 (e.g., a J591 PSMA binding domain). In some embodiments, the PSMA binding domain is a humanized PSMA binding domain. In some embodiments, the PSMA binding domain is a humanized PSMA-specific binding domain. In some embodiments, the PSMA binding domain is a humanized J591 PSMA binding domain. In some embodiments, the PSMA binding domain comprises any of the heavy and light chain variable regions disclosed in PCT Publication Nos. WO2017212250A1 and WO2018033749A1, the disclosures of which are hereby incorporated herein by reference in their entirety. For example, a PSMA binding domain of the present invention can comprise an scFv comprising any of the heavy and light chain variable regions disclosed therein. Accordingly, a PSMA-CAR of the present invention comprises a humanized version of the murine J591 antibody which binds human PSMA, as disclosed in WO2017212250A1 and WO2018033749A1. A CAR of the present disclosure may comprise a PSMA binding domain, any transmembrane domain, optionally any hinge domain, any costimulatory domain and any intracellular signaling domain as disclosed herein.

In another exemplary embodiment, the CAR of the invention comprises an antigen binding domain that binds to mucin-1 (MUC1). In certain embodiments, the antigen binding domain binds to a glycosylated form or glycoepitope of MUC1. In certain embodiments, the antigen binding domain is specific for a truncated glycoepitope of mucin-1 (Tn-MUC1). In certain embodiments, the antigen binding domain is specific for Tn-MUC1. In an exemplary embodiment, a CAR of the present disclosure having affinity for Tn-MUC1 on a target cell may comprise a Tn-MUC1 binding domain. In some embodiments, the Tn-MUC1 binding domain is a murine Tn-MUC1 binding domain, e.g., the Tn-MUC1 binding domain is of murine origin. In some embodiments, the Tn-MUC1 binding domain is a humanized Tn-MUC1 binding domain. In some embodiments, the Tn-MUC1 binding domain is a human Tn-MUC1 binding domain, e.g., the Tn-MUC1 binding domain is of human origin.

In some embodiments, the Tn-MUC1 binding domain is derived from the 5E5 antibody disclosed in PCT Publication No. WO2008/040362, the disclosure of which is incorporated herein by reference in its entirety. Accordingly, a CAR of the present disclosure comprises a Tn-MUC1 binding domain derived from the 5E5 antibody disclosed in PCT Publication No. WO2008/040362. In some embodiments, the Tn-MUC1 binding domain is a humanized Tn-MUC1 binding domain. In some embodiments, the humanized Tn-MUC1 binding domain is derived from any one of the humanized 5E5 heavy and light chain sequences disclosed in PCT Publication No. WO2015/159076, the disclosure of which is incorporated herein by reference in its entirety. Accordingly, a CAR of the present disclosure comprises a humanized Tn-MUC1 binding domain derived from any one of the humanized 5E5 heavy and light chain sequences disclosed in PCT Publication No. WO2015/159076. A CAR of the present disclosure may comprise a Tn-MUC1 binding domain, any transmembrane domain, optionally any hinge domain, any costimulatory domain and any intracellular signaling domain as disclosed herein.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof. In some embodiments, the antigen binding domain is selected from the group consisting of an antibody, an antigen binding fragment (Fab), and a single-chain variable fragment (scFv).

In some embodiments, a CAR of the present disclosure may have affinity for one or more target antigens on one or more target cells. In some embodiments, a CAR may have affinity for one or more target antigens on a single target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through a polypeptide linker, an Fc hinge region, or a membrane hinge region.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., Tn-MUC1 binding domain) comprises an scFv having the configuration from N-terminus to C-terminus, VH-linker-VL. In some embodiments, the antigen binding domain (e.g., a Tn-MUC1 binding domain, a PSMA binding domain) comprises an scFv having the configuration from N-terminus to C-terminus, VL-linker-VH. Those of skill in the art would be able to select the appropriate configuration for use in the present invention.

The linker is typically rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 1), (GGGS)ₙ (SEQ ID NO: 2), and (GGGGS)ₙ(SEQ ID NO: 3), where n represents an integer of at least 1. Exemplary linker sequences can comprise amino acid sequences including, without limitation, GGSG (SEQ ID NO: 4), GGSGG (SEQ ID NO: 5), GSGSG (SEQ ID NO: 6), GSGGG (SEQ ID NO: 7), GGGSG (SEQ ID NO: 8), GSSSG (SEQ ID NO: 9), GGGGS (SEQ ID NO: 3), GGGGSGGGGSGGGGS (SEQ ID NO: 10) and the like. Those of skill in the art would be able to select the appropriate linker sequence for use in the present invention. In one embodiment, an antigen binding domain (e.g., a Tn-MUC1 binding domain, a PSMA binding domain) of the present invention comprises a heavy chain variable region (VH) and a light chain variable region (VL), wherein the VH and VL is separated by the linker sequence having the amino acid sequence GGGGSGGGGSGGGGS (SEQ ID NO: 10), which may be encoded by a nucleic acid sequence comprising the nucleotide sequence ggtggcggtggctcgggcggtggtgggtcgggt ggcggcggatct (SEQ ID NO: 11).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Biol Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an SS bond for binding an antigen and where the remaining H chain portions are linked together. A “F(ab′)2” fragment can be split into two individual Fab′ fragments.

In some instances, the antigen binding domain may be derived from the same species in which the CAR will ultimately be used. For example, for use in humans, the antigen binding domain of the CAR may comprise a human antibody as described elsewhere herein, or a fragment thereof.

Transmembrane Domain

With respect to the transmembrane domain, the CAR of the present invention (e.g., a PSMA CAR, a Tn-MUC1 CAR) can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane regions of particular use in this invention include, without limitation, transmembrane domains derived from (i.e., comprise at least the transmembrane region(s) of) the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1B), CD154 (CD40L), CD278 (ICOS), CD357 (GITR), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

In some embodiments, the transmembrane domain further comprises a hinge region. A subject CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. In exemplary embodiments, the hinge region is capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 40 amino acids, or from about 40 amino acids to about 50 amino acids.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

For example, hinge regions include glycine polymers (G)n, glycine-serine polymers (including, for example, (GS)n, (GSGGS)n (SEQ ID NO: 1) and (GGGS)n (SEQ ID NO: 2), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO: 4), GGSGG (SEQ ID NO: 5), GSGSG (SEQ ID NO: 6), GSGGG (SEQ ID NO: 7), GGGSG (SEQ ID NO: 8), GSSSG (SEQ ID NO: 9), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO: 12); CPPC (SEQ ID NO: 13); CPEPKSCDTPPPCPR (SEQ ID NO: 14) (see, e.g., Glaser et al., J. Biol. Chem. (2005) 280:41494-41503); ELKTPLGDTTHT (SEQ ID NO: 15); KSCDKTHTCP (SEQ ID NO: 16); KCCVDCP (SEQ ID NO: 17); KYGPPCP (SEQ ID NO: 18); EPKSCDKTHTCPPCP (SEQ ID NO: 19) (human IgG1 hinge); ERKCCVECPPCP (SEQ ID NO: 20) (human IgG2 hinge); ELKTPLGDTTHTCPRCP (SEQ ID NO: 21) (human IgG3 hinge); SPNMVPHAHHAQ (SEQ ID NO: 22) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 23); see, e.g., Yan et al., J. Biol. Chem. (2012) 287: 5891-5897. In one embodiment, the hinge region can comprise an amino acid sequence derived from human CD8, or a variant thereof.

In one embodiment, the transmembrane domain comprises a CD8a transmembrane domain. In some embodiments, a subject CAR comprises a CD8a transmembrane domain comprising the amino acid sequence set forth in SEQ ID NO: 46, which may be encoded by a nucleic acid sequence comprising the nucleotide sequence set forth in SEQ ID NO: 47.

In another embodiment, a subject CAR comprises a CD8a hinge domain and a CD8a transmembrane domain. In one embodiment, the CD8a hinge domain comprises the amino acid sequence set forth in SEQ ID NO: 48, which may be encoded by a nucleic acid sequence comprising the nucleotide sequence set forth in SEQ ID NO: 49.

In one embodiment, the transmembrane domain comprises a CD28 transmembrane domain. In some embodiments, a subject CAR comprises a CD28 transmembrane domain comprising the amino acid sequence set forth in SEQ ID NO: 50, which may be encoded by a nucleic acid sequence comprising the nucleotide sequence set forth in SEQ ID NO: 51.

Tolerable variations of the transmembrane and/or hinge domain will be known to those of skill in the art, while maintaining its intended function. For example, in some embodiments a transmembrane domain or hinge domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 46, 48, and 50. For example, in some embodiments a transmembrane domain or hinge domain is encoded by a nucleic acid sequence comprising the nucleotide sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleotide sequences set forth in SEQ ID NO: 47, 49, and 51.

The transmembrane domain may be combined with any hinge domain and/or may comprise one or more transmembrane domains described herein.

The transmembrane domains described herein, such as a transmembrane region of alpha, beta or zeta chain of the T-cell receptor, CD28, CD2, CD3 epsilon, CD45, CD4, CD5, CD7, CD8, CD9, CD 16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9, can be combined with any of the antigen binding domains described herein, any of the costimulatory signaling domains or intracellular domains or cytoplasmic domains described herein, or any of the other domains described herein that may be included in the CAR.

In one embodiment, the transmembrane domain may be synthetic, in which case it will comprise predominantly hydrophobic residues such as leucine and valine. In exemplary embodiments, a triplet of phenylalanine, tryptophan and valine will be found at each end of a synthetic transmembrane domain.

In some embodiments, a subject CAR may further comprise, between the extracellular domain and the transmembrane domain of the CAR, or between the intracellular domain and the transmembrane domain of the CAR, a spacer domain. As used herein, the term “spacer domain” generally means any oligo- or polypeptide that functions to link the transmembrane domain to, either the extracellular domain or, the intracellular domain in the polypeptide chain. A spacer domain may comprise up to 300 amino acids, e.g., 10 to 100 amino acids, or 25 to 50 amino acids. In some embodiments, the spacer domain may be a short oligo- or polypeptide linker, e.g., between 2 and 10 amino acids in length. For example, glycine-serine doublet provides a particularly suitable linker between the transmembrane domain and the intracellular signaling domain of the subject CAR.

Accordingly, a subject CAR of the present disclosure may comprise any of the transmembrane domains, hinge domains, or spacer domains described herein.

Intracellular Domain

A subject CAR of the present invention also includes an intracellular domain. The intracellular domain of the CAR is responsible for activation of at least one of the effector functions of the cell in which the CAR is expressed (e.g., immune cell). The intracellular domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

In certain embodiments, the intracellular domain comprises a costimulatory signaling domain. In certain embodiments, the intracellular domain comprises an intracellular signaling domain. In certain embodiments, the intracellular domain comprises a costimulatory signaling domain and an intracellular signaling domain. In certain embodiments, the intracellular domain comprises 4-1BB and CD3 zeta. In certain embodiments, the costimulatory signaling domain comprises 4-1BB. In certain embodiments, the intracellular signaling domain comprises CD3 zeta.

In one embodiment, the intracellular domain of the CAR comprises a costimulatory signaling domain which includes any portion of one or more co-stimulatory molecules, such as at least one signaling domain from CD2, CD3, CD8, CD27, CD28, OX40, ICOS, 4-1BB, PD-1, any derivative or variant thereof, any synthetic sequence thereof that has the same functional capability, and any combination thereof.

Examples of the intracellular signaling domain include, without limitation, the (chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma R11a, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2Rbeta, IL2Rgamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Lyl08), SLAM (SLAMFI, CD150, PO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, Fc gamma RI, Fc gamma RIIA, Fc gamma RIIC, Fc gamma RIIIA, FcRL5 (see, e.g., Gillis et al., Front. (2014) Immunol. 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcR gamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). In one embodiment, an intracellular signaling domain suitable for use in a subject CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the costimulatory signaling domains described herein, any of the antigen binding domains described herein, any of the transmembrane domains described herein, or any of the other domains described herein that may be included in the CAR.

Further, variant intracellular signaling domains suitable for use in a subject CAR are known in the art. The YMFM motif is found in ICOS and is a SH2 binding motif that recruits both p85 and p50alpha subunits of PI3K, resulting in enhanced AKT signaling. See, e.g., Simpson et al. (2010) Curr. Opin. Immunol., 22:326-332. In one embodiment, a CD28 intracellular domain variant may be generated to comprise a YMFM motif.

In one embodiment, the intracellular domain of a subject CAR comprises a 4-1BB costimulatory domain comprising the amino acid sequence set forth in SEQ ID NO: 24, which may be encoded by a nucleic acid sequence comprising the nucleotide sequence set forth in SEQ ID NO: 25 or 26. In one embodiment, the intracellular domain of a subject CAR comprises a CD28 costimulatory domain comprising the amino acid sequence set forth in SEQ ID NO: 27, which may be encoded by a nucleic acid sequence comprising the nucleotide sequence set forth in SEQ ID NO: 28. In one embodiment, the intracellular domain of a subject CAR comprises a CD28(YMFM) costimulatory domain comprising the amino acid sequence set forth in SEQ ID NO: 29, which may be encoded by a nucleic acid sequence comprising the nucleotide sequence set forth in SEQ ID NO: 30. In one embodiment, the intracellular domain of a subject CAR comprises an ICOS costimulatory domain comprising the amino acid sequence set forth in SEQ ID NO: 31, which may be encoded by a nucleic acid sequence comprising the nucleotide sequence set forth in SEQ ID NO: 32 or 33. In one embodiment, the intracellular domain of a subject CAR comprises an ICOS(YMNM) costimulatory domain comprising the amino acid sequence set forth in SEQ ID NO: 34, which may be encoded by a nucleic acid sequence comprising the nucleotide sequence set forth in SEQ ID NO: 35. In one embodiment, the intracellular domain of a subject CAR comprises a CD2 costimulatory domain comprising the amino acid sequence set forth in SEQ ID NO: 36, which may be encoded by a nucleic acid sequence comprising the nucleotide sequence set forth in SEQ ID NO: 37. In one embodiment, the intracellular domain of a subject CAR comprises a CD27 costimulatory domain comprising the amino acid sequence set forth in SEQ ID NO: 38, which may be encoded by a nucleic acid sequence comprising the nucleotide sequence set forth in SEQ ID NO: 39. In one embodiment, the intracellular domain of a subject CAR comprises a OX40 costimulatory domain comprising the amino acid sequence set forth in SEQ ID NO: 40, which may be encoded by a nucleic acid sequence comprising the nucleotide sequence set forth in SEQ ID NO: 41.

In one embodiment, the intracellular domain of a subject CAR comprises a CD3 zeta intracellular signaling domain comprising the amino acid sequence set forth in SEQ ID NO: 42 or 44, which may be encoded by a nucleic acid sequence comprising the nucleotide sequence set forth in SEQ ID NO: 43 or 45, respectively.

Tolerable variations of the intracellular domain will be known to those of skill in the art, while maintaining specific activity. For example, in some embodiments the intracellular domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 24, 27, 29, 31, 34, 36, 38, 40, 42, and 44. For example, in some embodiments the intracellular domain is encoded by a nucleic acid sequence comprising a nucleotide sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleotide sequences set forth in SEQ ID NOs: 25, 26, 28, 30, 32, 33, 35, 37, 39, 41, 43, and 45.

In one embodiment, the intracellular domain of a subject CAR comprises an ICOS costimulatory domain and a CD3 zeta intracellular signaling domain. In one embodiment, the intracellular domain of a subject CAR comprises a CD28 costimulatory domain and a CD3 zeta intracellular signaling domain. In one embodiment, the intracellular domain of a subject CAR comprises a CD28 YMFM variant costimulatory domain and a CD3 zeta intracellular signaling domain. In one embodiment, the intracellular domain of a subject CAR comprises a CD27 costimulatory domain and a CD3 zeta intracellular signaling domain. In one embodiment, the intracellular domain of a subject CAR comprises a OX40 costimulatory domain and a CD3 zeta intracellular signaling domain. In one exemplary embodiment, the intracellular domain of a subject CAR comprises a 4-1BB costimulatory domain and a CD3 zeta intracellular signaling domain. In one exemplary embodiment, the intracellular domain of a subject CAR comprises a CD2 costimulatory domain and a CD3 zeta intracellular signaling domain.

Sequences of the domains of the CAR are found in Table 1.

TABLE 1

| SEQ ID | | |
|---|---|---|
| NO: | Description | Sequence |
| 24 | 4-1BB costimulatory | KRGRKKLLYIFKQPFMRPVQTTQEEDGCSCRFPEEEEGGCEL |
| | domain amino acid | |
| | sequence | |
| 25 | 4-1BB costimulatory | AAACGGGGCAGAAAGAAACTCCTGTATATATTCAAACAACCAT |
| | domain nucleic acid | TTATGAGACCAGTACAAACTACTCAAGAGGAAGACGGCTGTAG |
| | sequence | CTGCCGATTTCCAGAAGAAGAAGAAGGAGGATGTGAACTG |
| 26 | 4-1BB costimulatory | AAACGGGGCAGAAAGAAACTCCTGTATATATTCAAACAACCAT |
| | domain nucleic acid | TTATGAGACCAGTACAAACTACTCAAGAGGAAGATGGCTGTAG |
| | sequence | CTGCCGATTTCCAGAAGAAGAAGAAGGAGGATGTGAACTG |
| 27 | CD28 costimulatory | RSKRSRLLHSDYMNMTPRRPGPTRKHYQPYAPPRDFAAYRS |
| | domain amino acid | |
| | sequence | |
| 28 | CD28 costimulatory | aggagtaagaggagcaggctcctgcacagtgactacatgaacatgactccccgccgccc |
| | domain nucleic acid | cgggcccacccgcaagcattaccagccctatgccccaccacgcgacttcgcagcctatcg |
| | sequence | ctcc |
| 29 | CD28(YMFM) | RSKRSRLLHSDYMFMTPRRPGPTRKHYQPYAPPRDFAAYRS |
| | costimulatory domain | |
| | amino acid sequence | |
| 30 | CD28(YMFM) | AGGAGTAAGAGGAGCAGGCTCCTGCACAGTGACTACATGTTC |
| | costimulatory domain | ATGACTCCCCGCCGCCCCGGGCCCACCCGCAAGCATTACCA |
| | nucleic acid sequence | GCCCTATGCCCCACCACGCGACTTCGCAGCCTATCGCTCC |
| 31 | ICOS costimulatory | TKKKYSSSVHDPNGEYMFMRAVNTAKKSRLTDVTL |
| | domain amino acid | |
| | sequence | |
| 32 | ICOS costimulatory | ACAAAAAAGAAGTATTCATCCAGTGTGCACGACCCTAACGGTG |
| | domain nucleic acid | AATACATGTTCATGAGAGCAGTGAACACAGCCAAAAAATCCAG |
| | sequence | ACTCACAGATGTGACCCTA |
| 33 | ICOS costimulatory | ACAAAAAAGAAGTATTCATCCAGTGTGCACGACCCTAACGGTG |
| | domain nucleic acid | AATACATGTTCATGAGAGCAGTGAACACAGCCAAAAAATCTAG |
| | sequence | ACTCACAGATGTGACCCTA |
| 34 | ICOS(YMNM) | TKKKYSSSVHDPNGEYMNMRAVNTAKKSRLTDVTL |
| | costimulatory domain | |
| | amino acid sequence | |
| 35 | ICOS(YMNM) | ACAAAAAAGAAGTATTCATCCAGTGTGCACGACCCTAACGGTG |
| | costimulatory domain | AATACATGAACATGAGAGCAGTGAACACAGCCAAAAAATCCAG |
| | nucleic acid sequence | ACTCACAGATGTGACCCTA |
| 36 | CD2 costimulatory | TKRKKQRSRRNDEELETRAHRVATEERGRKPHQIPASTPQNPAT |
| | domain amino acid | SQHPPPPPGHRSQAPSHRPPPPGHRVQHQPQKRPPAPSGTQV |
| | sequence | HQQKGPPLPRPRVQPKPPHGAAENSLSPSSN |
| 37 | CD2 costimulatory | ACCAAAAGGAAAAAACAGAGGAGTCGGAGAAATGATGAGGAG |
| | domain nucleic acid | CTGGAGACAAGAGCCCACAGAGTAGCTACTGAAGAAAGGGGC |
| | sequence | CGGAAGCCCCACCAAATTCCAGCTTCAACCCCTCAGAATCCA |
| | | GCAACTTCCCAACATCCTCCTCCACCACCTGGTCATCGTTCCC |
| | | AGGCACCTAGTCATCGTCCCCCGCCTCCTGGACACCGTGTTC |
| | | AGCACCAGCCTCAGAAGAGGCCTCCTGCTCCGTCGGGCACAC |
| | | AAGTTCACCAGCAGAAAGGCCCGCCCCTCCCCAGACCTCGAG |
| | | TTCAGCCAAAACCTCCCCATGGGGCAGCAGAAAACTCATTGTC |
| | | CCCTTCCTCTAAT |
| 38 | CD27 costimulatory | QRRKYRSNKGESPVEPAEPCRYSCPREEEGSTIPIQEDYRKPEP |
| | domain amino acid | ACSP |
| | sequence | |
| 39 | CD27 costimulatory | CAACGAAGGAAATATAGATCAAACAAAGGAGAAAGTCCTGTGG |
| | domain nucleic acid | AGCCTGCAGAGCCTTGTCGTTACAGCTGCCCCAGGGAGGAG |
| | sequence | GAGGGCAGCACCATCCCCATCCAGGAGGATTACCGAAAACCG |
| | | GAGCCTGCCTGCTCCCCC |
| 40 | OX40 costimulatory | ALYLLRRDQRLPPDAHKPPGGGSFRTPIQEEQADAHSTLAKI |
| | domain amino acid | |
| | sequence | |
| 41 | OX40 costimulatory | GCCCTGTACCTGCTCCGCAGGGACCAGAGGCTGCCCCCCGA |
| | domain nucleic acid | TGCCCACAAGCCCCCTGGGGGAGGCAGTTTCAGGACCCCCAT |
| | sequence | CCAAGAGGAGCAGGCCGACGCCCACTCCACCCTGGCCAAGA |
| | | TC |
| 42 | CD3 zeta intracellular | RVKFSRSADAPAYQQGQNQLYNELNLGRREEYDVLDKRRGRDP |
| | signaling domain | EMGGKPRRKNPQEGLYNELQKDKMAEAYSEIGMKGERRRGKG |
| | amino acid sequence | HDGLYQGLSTATKDTYDALHMQALPPR |
| 43 | CD3 zeta intracellular | agagtgaagttcagcaggagcgcagacgcccccgcgtaccagcagggccagaaccag |
| | signaling domain | ctctataacgagctcaatctaggacgaagagaggagtacgatgttttggacaagagacgtg |
| | nucleic acid sequence | gccgggaccctgagatggggggaaagccgagaaggaagaaccctcaggaaggcctgt |
| | | acaatgaactgcagaaagataagatggcggaggcctacagtgagattgggatgaaaggc |
| | | gagcgccggaggggcaaggggcacgatggcctttaccagggtctcagtacagccaccaa |
| | | ggacacctacgacgcccttcacatgcaggccctgccccctcgc |
| 44 | CD3 zeta (Q14K) | RVKFSRSADAPAYKQGQNQLYNELNLGRREEYDVLDKRRGRDP |
| | intracellular signaling | EMGGKPRRKNPQEGLYNELQKDKMAEAYSEIGMKGERRRGKG |
| | domain amino acid | HDGLYQGLSTATKDTYDALHMQALPPR |
| | sequence | |
| 45 | CD3 zeta (Q14K) | AGAGTGAAGTTCAGCAGGAGCGCAGACGCCCCCGCGTACAA |
| | intracellular signaling | GCAGGGCCAGAACCAGCTCTATAACGAGCTCAATCTAGGACG |
| | domain nucleic acid | AAGAGAGGAGTACGATGTTTTGGACAAGAGACGTGGCCGGGA |
| | sequence | CCCTGAGATGGGGGGAAAGCCGAGAAGGAAGAACCCTCAGG |
| | | AAGGCCTGTACAATGAACTGCAGAAAGATAAGATGGCGGAGG |
| | | CCTACAGTGAGATTGGGATGAAAGGCGAGCGCCGGAGGGGC |
| | | AAGGGGCACGATGGCCTTTACCAGGGTCTCAGTACAGCCACC |
| | | AAGGACACCTACGACGCCCTTCACATGCAGGCCCTGCCCCCT |
| | | CGC |
| 46 | CD8 alpha | IYIWAPLAGTCGVLLLSLVITLYC |
| | transmembrane | |
| | domain amino acid | |
| | sequence | |
| 47 | CD8 alpha | atctacatctgggcgcccttggccgggacttgtggggtccttctcctgtcactggttatca |
| | transmembrane | ccctttactgc |
| | domain nucleic acid | |
| | sequence | |
| 48 | CD8 alpha hinge | TTTPAPRPPTPAPTIASQPLSLRPEACRPAAGGAVHTRGLDFACD |
| | domain amino acid | |
| | sequence | |
| 49 | CD8 alpha hinge | accacgacgccagcgccgcgaccaccaacaccggcgcccaccatcgcgtcgcagccc |
| | domain nucleic acid | ctgtccctgcgcccagaggcgtgccggccagcggcggggggcgcagtgcacacgaggg |
| | sequence | ggctggacttcgcctgtgat |
| 50 | CD28 transmembrane | FWVLVVVGGVLACYSLLVTVAFIIFWV |
| | domain amino acid | |
| | sequence | |
| 51 | CD28 transmembrane | ttttgggtgctggtggtggttggtggagtcctggcttgctatagcttgctagtaacagtgg |
| | domain nucleic acid | cctttattattttctgggtg |
| | sequence | |

### B. Modified Immune Cells

The present invention provides a modified immune cell or precursor cell thereof (e.g., a modified T cell, a modified NK cell, a modified NT cell), comprising a chimeric antigen receptor (CAR) and/or exogenous T cell receptor (TCR). Accordingly, such modified cells possess the specificity directed by the CAR and/or TCR that is expressed therein. For example, a modified cell of the present disclosure comprises a PSMA CAR and possesses specificity for PSMA on a target cell. For example, a modified cell of the present invention comprises a Tn-MIUC1 CAR and possesses specificity for a truncated glycoepitope of mucin-1 (Tn-MUC1) on a target cell. A modified immune cell comprising a Tn-MUC1 CAR is also referred to herein as CART-TnMUC1.

Any modified cell comprising a CAR comprising any antigen binding domain, any hinge, any transmembrane domain, any intracellular costimulatory domain, and any intracellular signaling domain described herein is envisioned, and can readily be understood and made by a person of skill in the art in view of the disclosure herein. Any modified cell comprising a TCR comprising affinity for any antigen (e.g., solid tumor antigen) is envisioned, and can readily be understood and made by a person of skill in the art in view of the disclosure herein.

In some embodiments, the modified cell is an immune cell or precursor cell thereof. In an exemplary embodiment, the modified cell is a T cell. In an exemplary embodiment, the modified cell is an autologous cell. In an exemplary embodiment, the modified cell is an autologous immune cell or precursor cell thereof. In an exemplary embodiment, the modified cell is an autologous T cell.

The present invention provides compositions and methods for modified immune cells or precursors thereof, e.g., modified T cells, comprising a dominant negative receptor and/or a switch receptor. Thus, in some embodiments, the immune cell has been genetically modified to express the dominant negative receptor and/or switch receptor. As used herein, the term “dominant negative receptor” refers to a molecule designed to reduce the effect of a negative signal transduction molecule, e.g., the effect of a negative signal transduction molecule on a modified immune cell of the present invention. A dominant negative receptor of the present invention may bind a negative signal transduction molecule, e.g., TGF-β or PD-1, by virtue of an extracellular domain associated with the negative signal, and reduce the effect of the negative signal transduction molecule. Such dominant negative receptors and switch receptors are described in U.S. Patent Application Ser. No. 62/639,321, or PCT Patent Application No. PCT/US2019/020729, the disclosures of which are incorporated herein by reference in its entirety. For example, a modified immune cell comprising a dominant negative receptor may bind a negative signal transduction molecule in the microenvironment of the modified immune cell, and reduce the effect the negative signal transduction molecule may have on the modified immune cell.

A switch receptor of the present invention may be designed to, in addition to reducing the effects of a negative signal transduction molecule, to convert the negative signal into a positive signal, by virtue of comprising an intracellular domain associated with the positive signal. Switch receptors designed to convert a negative signal into a positive signal are described herein. Accordingly, switch receptors comprise an extracellular domain associated with a negative signal and/or an intracellular domain associated with a positive signal.

Tumor cells generate an immunosuppressive microenvironment that serves to protect them from immune recognition and elimination. This immunosuppressive microenvironment can limit the effectiveness of immunosuppressive therapies such as CAR-T cell therapy. The secreted cytokine Transforming Growth Factor β (TGFβ) directly inhibits the function of cytotoxic T cells and additionally induces regulatory T cell formation to further suppress immune responses. T cell immunosuppression due to TGFβ in the context of prostate cancers has been previously demonstrated (Donkor et al., 2011; Shalapour et al., 2015). To reduce the immunosuppressive effects of TGFβ, immune cells can be modified to express a dominant negative receptor that is a dominant negative receptor for TGF-β.

In some embodiments, modified cells of the present disclosure comprise a dominant negative receptor comprising a truncated variant of a wild-type protein associated with a negative signal. In some embodiments, the dominant negative receptor is a dominant negative receptor for TGF-β. Accordingly, in some embodiments, the dominant negative receptor for TGF-β is a truncated variant of a wild-type TGF-β receptor. In some embodiments, the dominant negative receptor is a truncated dominant negative variant of the TGF-β receptor type II (TGFβRII-DN). TGFβRII-DN is described in U.S. Patent Application Ser. No. 62/639,321, or PCT Patent Application No. PCT/US2019/020729, the disclosures of which are incorporated herein by reference in its entirety. Accordingly, in certain exemplary embodiments, a modified immune cell of the present disclosure comprises a PSMA-CAR and a truncated dominant negative variant of the TGF-β receptor type II (TGFβRII-DN). A modified cell comprising a PSMA-CAR and a truncated dominant negative variant of the TGF-β receptor type II (TGFβRII-DN) is also referred to herein as CART-PSMA-TGFβRDN.

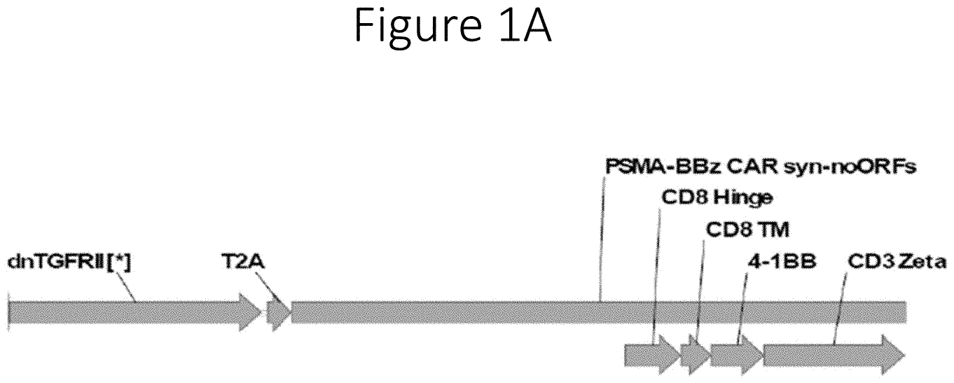

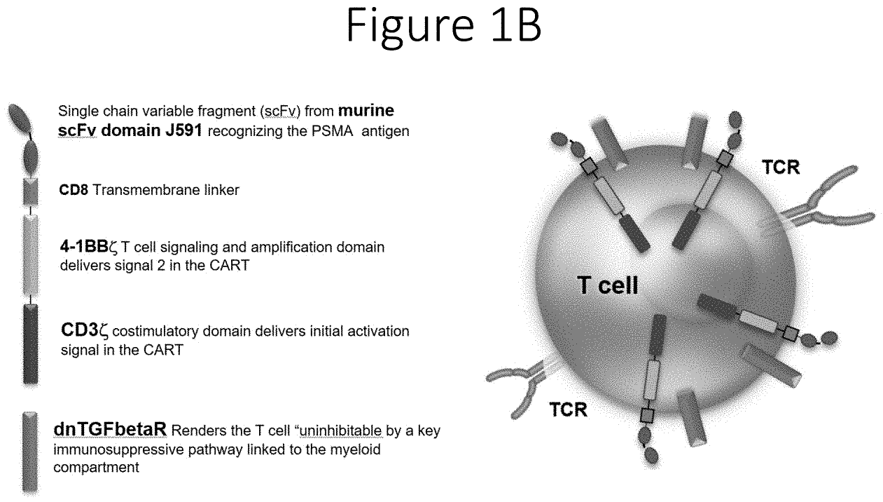

In certain exemplary embodiments a modified cell of the present disclosure is CART-PSMA-TGFβRDN, an autologous T-cell are transduced by lentivirus vector to express a dominant negative variation of the type II transforming growth factor beta receptor (TGFβRDN) and a second-generation chimeric antigen receptor (CAR) specific to the prostate specific membrane antigen (PSMA) protein. The TGFβRDN transgene has been generated by truncation of a cytoplasmic portion of type II transforming growth factor beta receptor (TGFβRII) and the CAR construct has been generated by fusion of a PSMA-specific single-chain variable region fragment (scFv) with human CD3ζ and 4-1BB signaling domains. The anti-PSMA scFv was derived from the murine J591 anti-PSMA antibody. The construct is bicistronic, which allows nearly equivalent expression of both transgenes from the same vector. The CAR construct used for CART-PSMA-TGFβRDN is shown in FIG. 1A and the structure of the CAR of CART-PSMA-TGFβRDN is shown in FIG. 1B.

### C. Nucleic Acids and Expression Vectors

The present invention provides a nucleic acid encoding a CAR (e.g. a PSMA CAR, a Tn-MUC1 CAR), TCR, and/or a truncated dominant negative variant of the TGF-β receptor type II. As described herein, a CAR comprises an antigen binding domain (e.g., a PSMA binding domain, a MUC1 binding domain), a transmembrane domain, and an intracellular domain. Accordingly, the present invention provides a nucleic acid encoding an antigen binding domain (e.g., a PSMA binding domain, a MUC1 binding domain), a transmembrane domain, and an intracellular domain of a CAR.

In some embodiments, a nucleic acid of the present disclosure may be operably linked to a transcriptional control element, e.g., a promoter, and enhancer, etc. Suitable promoter and enhancer elements are known to those of skill in the art.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and P Lambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the EF-1 alpha promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR and/or TCR inducible expression cassette. In one embodiment, the CAR and/or TCR inducible expression cassette is for the production of a transgenic polypeptide product that is released upon CAR and/or TCR signaling. See, e.g., Chmielewski and Abken, Expert Opin. Biol. Ther. (2015) 15(8): 1145-1154; and Abken, Immunotherapy (2015) 7(5): 535-544.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in any way as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., murine leukemia virus, spleen necrosis virus, and vectors derived from retroviruses such as Rous sarcoma virus, Harvey sarcoma virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the CAR, TCR, and/or a truncated dominant negative variant of the TGF-0 receptor type II into an immune cell or precursor thereof (e.g., a T cell; CART-TnMUC1; CART-PSMA-TGFβRDN). Accordingly, an expression vector (e.g., a lentiviral vector) of the present invention may comprise a nucleic acid encoding a CAR, TCR, and/or a truncated dominant negative variant of the TGF-0 receptor type II. In some embodiments, the expression vector (e.g., lentiviral vector) will comprise additional elements that will aid in the functional expression of the CAR, TCR, and/or a truncated dominant negative variant of the TGF-0 receptor type II encoded therein. In some embodiments, an expression vector comprising a nucleic acid encoding a CAR, TCR, and/or a truncated dominant negative variant of the TGF-0 receptor type II further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR, TCR, and/or a truncated dominant negative variant of the TGF-0 receptor type II encoding nucleic acid sequence). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., a lentiviral vector) are known to those of skill in the art and may be incorporated into a vector of the present invention. In some embodiments, the vector (e.g., a lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., a lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR, TCR, and/or a truncated dominant negative variant of the TGF-β receptor type II.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a CAR, TCR, and/or a truncated dominant negative variant of the TGF-β receptor type II of the present disclosure. Methods for introducing RNA into a host cell are known in the art. See, e.g., Zhao et al. Cancer Res. (2010) 15: 9053. Introducing RNA comprising a nucleotide sequence encoding a CAR, TCR, and/or a truncated dominant negative variant of the TGF-β receptor type II of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a CAR, TCR, and/or a truncated dominant negative variant of the TGF-β receptor type II of the present disclosure.

To assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

### D. Methods of Generating Modified Immune Cells

The present invention provides methods for producing/generating a modified immune cell or precursor cell thereof (e.g., a T cell/NK cell/NKT cell). The cells are generally engineered by introducing a nucleic acid encoding a CAR, TCR, and/or a truncated dominant negative variant of the TGF-β receptor type II (e.g., CART-TnMUC1; CART-PSMA-TGFβRDN).

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In some embodiments, a nucleic acid encoding a CAR, TCR, and/or a truncated dominant negative variant of the TGF-β receptor type II of the invention is introduced into a cell by an expression vector. Expression vectors comprising a nucleic acid encoding a CAR, TCR, and/or a truncated dominant negative variant of the TGF-β receptor type II (e.g., CART-TnMUC1; CART-PSMA-TGFβRDN) are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the CAR, TCR, and/or a truncated dominant negative variant of the TGF-β receptor type II in the host cell. In some embodiments, the adenovirus genome is a 36 kb, linear, double stranded DNA, where a foreign DNA sequence (e.g., a nucleic acid encoding a CAR, TCR, and/or a truncated dominant negative variant of the TGF-β receptor type II) may be inserted to substitute large pieces of adenoviral DNA in order to make the expression vector of the present invention (see, e.g., Danthinne and Imperiale, Gene Therapy (2000) 7(20): 1707-1714).

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a CAR, TCR, and/or a truncated dominant negative variant of the TGF-β receptor type II) into the viral genome at certain locations to produce a virus that is replication defective. Though the retrovirus vectors are able to infect a broad variety of cell types, integration and stable expression of the CAR, TCR, and/or a truncated dominant negative variant of the TGF-β receptor type II, requires the division of host cells.

Lentivirus vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the human immunodeficiency viruses (HIV-1, HIV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a CAR, TCR, and/or a truncated dominant negative variant of the TGF-β receptor type II (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof, e.g., a T cell, an NK cell, or an NKT cell.

The present invention also provides genetically engineered cells which include and stably express a CAR, TCR, and/or a truncated dominant negative variant of the TGF-β receptor type II. In some embodiments, the genetically engineered cells are genetically engineered T-lymphocytes (T cells), regulatory T cells (Tregs), naive T cells (TN), memory T cells (for example, central memory T cells (TCM), effector memory cells (TEM)), natural killer cells (NK cells), natural killer T cells (NKT cells) and macrophages capable of giving rise to therapeutically relevant progeny. In one embodiment, the genetically engineered cells are autologous cells.

Modified cells (e.g., comprising a CAR, TCR, and/or a truncated dominant negative variant of the TGF-β receptor type II; CART-TnMUC1; CART-PSMA-TGFβRDN) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a CAR, TCR, and/or a truncated dominant negative variant of the TGF-β receptor type II of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

RNA

In one embodiment, the nucleic acids introduced into the host cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5′ to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA typically has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In certain exemplary embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA is electroporated into the cells, such as in vitro transcribed RNA.

The disclosed methods can be applied to the modulation of host cell activity in basic research and therapy, in the fields of cancer, stem cells, acute and chronic infections, and autoimmune diseases, including the assessment of the ability of the genetically modified host cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of host cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this non-physiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatuses for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

Accordingly, the present invention provides a method for generating a modified immune cell or precursor cell thereof comprising introducing into the cell an isolated nucleic acid (e.g., an expression construct) encoding for a CAR, TCR, and/or a truncated dominant negative variant of the TGF-β receptor type II as described herein, using any of the delivery methods described herein or are known to those of skill in the art.

### E. Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. In certain exemplary embodiments, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells and/or NKT cells. Other exemplary cells include stem cells, such as multipotent and pluripotent stem cells, including induced pluripotent stem cells (iPSCs). In certain aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a natural killer T cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in certain aspects, contain lymphocytes, including T cells, monocytes, granulocytes, B cells, other nucleated white blood cells, red blood cells, and/or platelets, and in certain aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some certain, a washing step is accomplished by tangential flow filtration (TFF) according to the manufacturer's instructions. In certain embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or another saline solution with or without buffer. In some embodiments, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in certain aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In certain aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In certain exemplary embodiments, multiple rounds of separation steps are carried out, where the positively or negatively selected fraction from one step is subjected to another separation step, such as a subsequent positive or negative selection. In certain exemplary embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (markerhigh) of one or more particular markers, such as surface markers, or that are negative for (marker−) or express relatively low levels (markerlow) of one or more markers. For example, in certain aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In certain exemplary embodiments, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In certain aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in certain aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L−CD8+ and/or CD62L+CD8+ fractions, such as using anti-CD8 and anti-CD62L antibodies. In some embodiments, a CD4+ T cell population and/or a CD8+T population is enriched for central memory (TCM) cells. In some embodiments, the enrichment for central memory T (TCM) cells is based on positive or high surface expression of CD45RO, CD62L, CCR7, CD28, CD3, and/or CD 127; in certain aspects, it is based on negative selection for cells expressing or highly expressing CD45RA and/or granzyme B. In certain aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in certain aspects are carried out simultaneously and in other aspects are carried out sequentially, in either order. In some embodiments, the same CD4 expression-based selection step used in preparing the CD8+ cell population or subpopulation, also is used to generate the CD4+ cell population or subpopulation, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L- and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In certain aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. An exemplary method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### F. Expansion of Immune Cells

Whether prior to or after modification of cells to express a CAR, TCR, and/or a truncated dominant negative variant of the TGF-β receptor type II, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the immune cells of the invention may be expanded by contact with a surface having attached thereto an agent that stimulates a CD3/TCR complex associated signal and a ligand that stimulates a co-stimulatory molecule on the surface of the immune cells. In particular, immune cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the immune cells, a ligand that binds the accessory molecule is used. For example, immune cells can be contacted with an anti-CD3 antibody and an anti-CD28 antibody, under conditions appropriate for stimulating proliferation of the immune cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding the immune cells by the methods disclosed herein can be multiplied by about 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700 fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater, and any and all whole or partial integers therebetween. In one embodiment, the immune cells expand in the range of about 20-fold to about 50-fold.

Following culturing, the immune cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. In certain exemplary embodiments, the level of confluence is 70% or greater before passing the cells to another culture apparatus. In particularly exemplary embodiments, the level of confluence is 90% or greater. A period of time can be any time suitable for the culture of cells in vitro. The immune cell medium may be replaced during the culture of the immune cells at any time. In certain exemplary embodiments, the immune cell medium is replaced about every 2 to 3 days. The immune cells are then harvested from the culture apparatus whereupon the immune cells can be used immediately or cryopreserved to be stored for use at a later time. In one embodiment, the invention includes cryopreserving the expanded immune cells. The cryopreserved immune cells are thawed prior to introducing nucleic acids into the immune cell.

In another embodiment, the method comprises isolating immune cells and expanding the immune cells. In another embodiment, the invention further comprises cryopreserving the immune cells prior to expansion. In yet another embodiment, the cryopreserved immune cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of immune cells comprises the addition to the cellular growth factors, such as those described in U.S. Pat. No. 5,199,942, or other factors, such as flt3-L, IL-1, IL-3 and c-kit ligand. In one embodiment, expanding the immune cells comprises culturing the immune cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging. Therefore, the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

The medium used to culture the immune cells may include an agent that can co-stimulate the immune cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater. In one embodiment, the immune cells expand in the range of about 2-fold to about 50-fold, or more by culturing the electroporated population. In one embodiment, human T regulatory cells are expanded via anti-CD3 antibody coated KT64.86 artificial antigen presenting cells (aAPCs). Methods for expanding and activating immune cells can be found in U.S. Pat. Nos. 7,754,482, 8,722,400, and 9,555,105, the contents of which are incorporated herein in their entirety.

In one embodiment, the method of expanding the immune cells can further comprise isolating the expanded immune cells for further applications. In another embodiment, the method of expanding can further comprise a subsequent electroporation of the expanded immune cells followed by culturing. The subsequent electroporation may include introducing a nucleic acid encoding an agent, such as a transducing the expanded immune cells, transfecting the expanded immune cells, or electroporating the expanded immune cells with a nucleic acid, into the expanded population of immune cells, wherein the agent further stimulates the immune cell. The agent may stimulate the immune cells, such as by stimulating further expansion, effector function, or another immune cell function.

### G. Methods of Treatment

Provided are methods and compositions for use in cell therapy, for the treatment of diseases or conditions including various tumors. The methods involve administering engineered cells (e.g., immune cells or precursors thereof; CART-TnMUC1; CART-PSMA-TGFβRDN) expressing recombinant receptors designed to recognize and/or specifically bind to molecules associated with the disease or condition and result in a response, such as an immune response against such molecules upon binding to such molecules.

In some embodiments, the recombinant receptor is a chimeric antigen receptor (CAR) and/or T cell receptor (TCR). In some embodiments, the CAR is directed to PSMA. In some embodiments, the CAR is directed to MUC1 (e.g., Tn-MUC1). In some embodiments, the recombinant receptor is a T cell receptor (TCR).

In some embodiments, the engineered cells comprise a CAR, TCR, and/or a truncated dominant negative variant of the TGF-β receptor type II. In certain embodiments, the engineered cells comprise a PSMA-CAR and/or a truncated dominant negative variant of the TGF-β receptor type II. In certain embodiments, the engineered cells comprise a PSMA-CAR and a truncated dominant negative variant of the TGF-β receptor type II. In certain embodiments, the engineered cell is CART-PSMA-TGFβRDN. In certain embodiments, the engineered cells comprise a TnMUC1-1 CAR. In certain embodiments, the engineered cell is CART-TnMUC1.

In particular, the methods provided herein may employ a fractionated dosing strategy to administer one or more doses of the engineered cells. The doses generally are administered in particular amounts and according to particular timing parameters. In some embodiments, the methods generally involve administering a first dose of cells to assess safety, followed by a consecutive dose of cells. In some embodiments, the number of cells administered and timing of the multiple doses are designed to reduce the likelihood or degree of toxicity to the subject.

Delivering high initial doses of engineered cells does not necessarily increase exposure. Particularly in the context of solid tumors, administering large doses does not necessarily enhance efficacy and can lead to increased or rapid expansion of the cells and result in toxicity. Certain reports have indicated a lack of correlation between dose and toxicity. See Park et al, Molecular Therapy 15(4):825-833 (2007). Higher initial doses can promote toxic outcomes such as cytokine release syndrome (CRS). High doses do not necessarily translate to increased persistence of the administered cells. See Park et al, Molecular Therapy 15(4):825-833 (2007).

The methods provided herein are aimed at addressing the development or degree of toxicity that may occur upon administration of the engineered cells. In some embodiments, the toxicity may be alleviated with the use of agents that target the downstream effects of toxicity, such as cytokine blockade, and/or delivering agents such as high-dose steroids which can also eliminate or impair the function of administered cells. Many of these approaches do not prevent other forms of toxicity such as neurotoxicity, which can be associated with adoptive cell therapy.

Provided herein are methods employing the fractionated administration of a total dose of engineered cells (e.g., CAR-T cells; CART-TnMUC1; CART-PSMA-TGFβRDN) in a way that minimizes risk of toxicity (e.g., on-target off-tumor toxicity). In some embodiments, the fractionated dosing strategy comprises a portion of the total dose administered on day 0, and the remainder of the full dose administered on days 5 to 7.

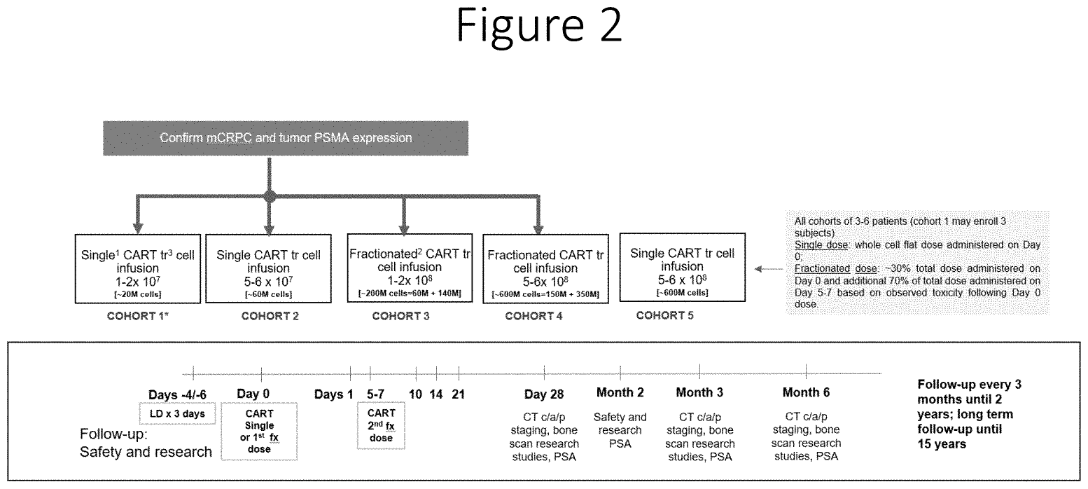

FIG. 2 provides an overview of the engineered cell (e.g., CAR-T; CART-TnMUC1; CART-PSMA-TGFβRDN) dosing schedule together with a lymphodepleting dosing regimen.

In certain embodiments, the present disclosure provides a method of treating a solid tumor in a subject comprising administering to the subject a first dose of cells (e.g., CAR-T cells; CART-TnMUC1; CART-PSMA-TGFβRDN) comprising a fractionated dosing regimen of a total dose of cells. As used herein, “fractionated dosing,” or a “fractionated dose” refers to a dosing regimen comprising a first dose comprising a fraction of the total dose of the cells, and a consecutive dose comprising the remainder of the total dose of the cells. In certain embodiments, the present disclosure provides a method of treating a solid tumor in a subject comprising administering to the subject a first dose of cells (e.g., CAR-T cells; CART-TnMUC1; CART-PSMA-TGFβRDN) comprising about 30% of a total dose of cells; and administered to the subject a consecutive dose of cells comprising about 70% of the total dose of cells. In some embodiments, the method comprises administering to the subject a first dose of cells, wherein the cells comprise a chimeric antigen receptor (CAR) having affinity for a solid tumor antigen, and wherein the first dose comprises about 30% of a total dose of cells; and administering to the subject a consecutive dose of cells comprising the CAR, wherein the consecutive dose comprises about 70% of the total dose of cells.

For all of the method described herein, the first dose of cells can alternatively comprise about 10%, about 15%, about 20%, about 25%, about 30%, about 35%, about 40%, about 45%, or about 50% of a total dose of cells, with the consecutive dose therefore comprising about 90%, about 85%, about 80%, about 75%, about 70%, about 65%, about 60%, about 55%, or about 50%, respectively, of the total dose of cells. In other aspects, the first dose of cells can be in a range, e.g., about 25% to about 35%, or any amount in-between these two values, of a total dose of cells, with the consecutive dose therefore comprising about 75% to about 65%, or any amount in-between these two values.

In some embodiments, the consecutive dose is administered five days after the administration of the first dose. In some embodiments, the consecutive dose is administered six days after the administration of the first dose. In some embodiments, the consecutive dose is administered seven days after the administration of the first dose. In some embodiments, the consecutive dose is administered five to seven after the administration of the first dose. In some embodiments, the consecutive dose is administered at least five days after the administration of the first dose. In some embodiments, the consecutive dose is administered at least six days after the administration of the first dose. In some embodiments, the consecutive dose is administered at least seven days after the administration of the first dose.

As used herein, a “total dose” of cells refers to the total effective amount of engineered cells (e.g., CAR-T cells; CART-TnMUC1; CART-PSMA-TGFβRDN). In the case where the subject is administered a fractionated dose, a first dose may comprise about 30% of the total dose of cells, and the consecutive dose may comprise the remainder of the total dose of cells, i.e., about 70% of the total dose of cells. In some embodiments, the total dose of cells is 1×10⁷/m² to 3×10⁷/m². In some embodiments, the total dose of cells is 1×10⁸/m² to 3×10⁸/m². In some embodiments, the total dose of cells is 1×10⁷/m² to 2×10⁷/m². In some embodiments, the total dose of cells is 5×10⁷/m² to 6×10⁷/m². In some embodiments, the total dose of cells is 1×10⁸/m² to 2×10⁸/m². In some embodiments, the total dose of cells is 5×10⁸/m² to 6×10⁸/m². In some embodiments, the total dose of cells is from about 10,000,000 cells/m² to about 600,000,000 cells/m², e.g., about 8,000,000 cells/m², about 9,000,000 cells/m², about 10,000,000 cells/m², about 11,000,000 cells/m², about 12,000,000 cells/m², about 15,000,000 cells/m², about 20,000,000 cells/m², about 30,000,000 cells/m², about 40,000,000 cells/m², about 50,000,000 cells/m², about 60,000,000 cells/m², about 80,000,000 cells/m², about 90,000,000 cells/m², about 100,000,000 cells/m², about 200,000,000 cells/m², about 300,000,000 cells/m², about 400,000,000 cells/m², about 500,000,000 cells/m², about 600,000,000 cells/m², about 700,000,000 cells/m², or more, and any value in between.

In certain embodiments, a fractionated dosing regimen is employed, comprising a first dose of about 30% of any of the total doses of cells as described herein, and a consecutive dose comprising the remainder (i.e., about 70%) of the total dose of cells. In some embodiments, a first dose comprises about 6×10⁷ cells/m² and a consecutive dose comprises about 1.4×10⁸ cells/m², resulting in a total dose of about 2×10⁸ cells/m². In some embodiments, a first dose comprises about 3×10⁷ cells/m² and a consecutive dose comprises about 7×10⁷ cells/m², resulting in a total dose of about 1×10⁸ cells/m². In some embodiments, a first dose comprises about 1.8×10⁸ cells/m² and a consecutive dose comprises about 4.2×10⁸ cells/m², resulting in a total dose of about 6×10⁸ cells/m². In some embodiments, a first dose comprises about 1.5×10⁸ cells/m² and a consecutive dose comprises about 3.5×10⁸ cells/m², resulting in a total dose of about 5×10⁸ cells/m².

The provided methods generally involve administering multiple doses of cells expressing recombinant receptors, such as CARs, or other antigen receptors, such as transgenic TCRs, to subjects having a disease or condition specifically recognized by the receptors. The administrations generally effect an improvement in one or more symptoms of the disease or condition and/or treat or prevent the disease or condition or symptom thereof.

As used herein, a “subject” may be a mammal, such as a human or other animal, and typically is human. In some embodiments, the subject has been treated with a therapeutic agent targeting the disease or condition, e.g. the tumor, prior to administration of the first dose and/or prior to the administration of the consecutive dose. In some embodiments, the subject is refractory to the other therapeutic agent.

In some embodiments, the subject is responsive to the other therapeutic agent, and treatment with the therapeutic agent ameliorates the disease. In some embodiments, the subject exhibits a relapse of the disease or condition over time (e.g., the subject responds favorable to an initial treatment, and is in remission, but exhibits a relapse in the disease). In some embodiments, the subject has not relapsed. In some embodiments, subjects that have been determined to be at a high risk of relapse, are administered the engineered cells prophylactically, e.g., to reduce the likelihood of relapse, or to prevent relapse.

In some embodiments, the subject has persistent or relapsed disease, e.g., following treatment with another therapeutic intervention. In some embodiments, the therapeutic intervention includes, e.g., radiation, chemotherapy, hematopoietic stem cell transplantation (HSCT), e.g., allogeneic HSCT or autologous HSCT. In some embodiments, the administration effectively treats the subject despite the subject having become resistant to another therapy.

In some embodiments, the subject has not received prior treatment with another therapeutic agent.

Among the diseases, conditions, and disorders are tumors, including solid tumors, melanomas, and hematologic malignancies, and includes metastatic and/or localized tumors, infectious diseases, such as infection with a virus or other pathogen, e.g., HBV, HCV, HIV, CMV, and parasitic disease, and inflammatory and autoimmune diseases. In some embodiments, the disease or condition is a tumor, cancer, neoplasm, malignancy, or other proliferative disease or disorder. Such diseases include but are not limited to, e.g., in the case of a PSMA targeted therapy: prostate cancer, and metastatic castrate resistant prostate cancer; in the case of a Tn-MUC1 targeted therapy: lung cancer, non-small cell lung cancer, breast cancer, triple negative breast cancer, pancreatic cancer, pancreatic adenocarcinoma, ovarian cancer, and fallopian tube cancer.

Methods for administration of cells for adoptive cell therapy are known and may be used in connection with the provided methods and compositions. For example, adoptive T cell therapy methods are described, e.g., in US Patent Application Publication No. 2003/0170238; U.S. Pat. No. 4,690,915; Rosenberg (2011) Nat Rev Clin Oncol. 8(10):577-85. See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some embodiments, the cells are isolated from a subject, and after engineering and processing, the cells are administered to the same subject. In some embodiments, the cell therapy is carries out by allogeneic transfer, in which the cells are isolated and/or otherwise prepared from a different subject than the subject who is to receive the cell therapy. In some embodiments, the subject from which the cells are isolated and the subject receiving the therapy is genetically similar. In some embodiments, the subject from which the cells are isolated and the subject receiving the therapy express the same HLA class or supertype.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, another therapeutic intervention. In some embodiments, the sequential administration can occur in any order. In some embodiment, the other therapeutic intervention may include, e.g., an antibody, a cytokine (e.g., IL-2, IL-15, IL-7, and the like), an agent (e.g., a cytotoxic or therapeutic agent), an engineered cell, or a fusion polypeptide (e.g., chimeric receptor). In some embodiments, the engineered cells are co-administered with the other therapeutic intervention. Such co-administration may be performed close in time such that the engineered cells and other therapeutic intervention enhance the efficacy of each other. In some embodiments, the engineered cells are administered prior to the other therapeutic intervention. In some embodiments, the engineered cells are administered after the other therapeutic intervention.

Preconditioning subjects with immunodepleting (e.g., lymphodepleting) therapies can improve the effects of adoptive cell therapy (ACT). Thus, in some embodiments, the methods include administering a preconditioning agent, such as a lymphodepleting or chemotherapeutic agent, such as cyclophosphamide, fludarabine, or combinations thereof, to a subject prior to the first or subsequent dose. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the first or subsequent dose.

In some embodiments, where the lymphodepleting agent comprises cyclophosphamide, the subject is administered a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day. In an exemplary embodiment, the dosing of cyclophosphamide is 300 mg/m²/day over three days, and the dosing of fludarabine is 30 mg/m²/day over three days.

In some embodiments, the lymphodepleting agent comprises about 300 mg/m²/day cyclophosphamide. In some embodiments, the lymphodepleting agent comprises about 30 mg/m²/day fludarabine. In some embodiments, the lymphodepleting agent comprises about 300 mg/m²/day cyclophosphamide and about 30 mg/m²/day fludarabine. In some embodiments, the lymphodepleting agent is administered to the subject between days −6 to −4. In some embodiments, the lymphodepleting agent is administered to the subject consecutively at days −6, −5, and −4. In some embodiments, the lymphodepleting agent comprises about 300 mg/m²/day cyclophosphamide and about 30 mg/m²/day fludarabine administered to the subject consecutively at days −6, −5, and −4.

In some embodiments, the administration of the preconditioning agent prior to infusion of the first or subsequent dose improves an outcome of the treatment. For example, in some embodiments, preconditioning improves the efficacy of treatment with the first or subsequent dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the first or subsequent dose. In some embodiments, the time to median disease-free survival is increased.

Once the cells are administered to the subject (e.g., human), the biological activity of the engineered cell populations in some embodiments is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107α, IFNγ, IL-2, and TNF. In some embodiments, the biological activity is measured by assessing clinical outcome, such as reduction in tumor burden or load. In some embodiments, toxic outcomes, persistence and/or expansion of the cells, and/or presence or absence of a host immune response, are assessed.

In some embodiments, a fractionated dosing regimen is designed to adapt the treatment to reduce and/or manage the risk of toxicity. In some embodiments, the fractionated dosing regimen is employed to reduce and/or manage the risk and/or development of a non-target mediated toxicity (e.g., cytokine release syndrome (CRS) or CAR-related encephalopathy syndrome (CRES). In some embodiments, the fractionated dosing regimen is employed to reduce and/or manage the risk and/or development of an on-target off-tumor toxicity. In some embodiments, the fractionated dosing regimen is employed to reduce and/or manage the risk and/or development of a non-target mediated toxicity and an on-target off-tumor toxicity. In some embodiments, the fractionated dosing regimen is employed to reduce and/or manage the risk and/or development of cytokine release syndrome (CRS), immune cell-associated neurologic toxicities, and/or on-target off-tumor toxicity.

In certain embodiments, the methods provided herein further comprise monitoring the development of cytokine release syndrome resulting from the administration of a first dose of engineered cells (e.g., CAR-T cells). It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS) (grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the present disclosure provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

A subset of patients with CRS may manifest symptoms similar to macrophage activation syndrome (MAS) or hemophagocytic lymphohistiocytosis (HLH). Features consistent with macrophage activation syndrome (MAS) or hemophagocytic lymphohistiocytosis (HLH) have been observed in patients treated with CAR-T therapy, coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity. See, e.g., Namuduri and Brentjens, Expert Rev. Hematol. (2016) 9(6): 511-513, hereby incorporated by reference in its entirety.

In certain embodiments, the methods provided herein further comprise monitoring the development of immune cell-associated neurological toxicities resulting from the administration of a first dose of engineered cells (e.g., CAR-T cells; CART-TnMUC1; CART-PSMA-TGFβRDN).

In some embodiments, immune cell-associated neurological toxicity includes CAR-related encephalopathy syndrome (CRES). Accordingly, the present disclosure provides for, following the diagnosis of CRES, appropriate CRES management strategies to mitigate the physiological symptoms of CRES. CRES management strategies are known in the art. For example, benzodiazepines (e.g., lorazepam) may be administered to control seizures that may arise from severe CRES-related impairment. Immune effector cell-associated neurotoxicity syndrome (ICANS) may manifest as delirium, encephalopathy, aphasia, lethargy, difficulty concentrating, agitation, tremor, seizures, and, rarely, cerebral edema. In addition, headache is very common and might not represent neurotoxicity per se. Previously considered in aggregate with CRS, neurotoxicity is now treated as a separate entity owing to its distinct timing and response to intervention. Neurologic symptoms may occur during or more commonly after CRS symptoms, vary among patients, and have an unclear pathophysiology, distinct from CRS. See, e.g., Lee et al. Biology of Blood and Marrow Transplantation (2019) 25(4): 625-538, hereby incorporated by reference in its entirety.

Mild to moderate cases of CRES are generally managed with supportive care (e.g., I.V. hydration and limiting oral intake), neurology evaluation and consultation (e.g., EEG, fundoscopic exam, brain/spine MRI), and tocilizumab and early corticosteroid therapy may be considered (e.g., 8 mg/kg I.V. maximum dose 800 mg Q8h of tocilizumab; and/or dexamethasone 10 mg I.V. Q6h). For severe CRES impairment, additional treatment with methylprednisolone (e.g., 1 mg/kg I.V. Q12h), and status epilepticus management may be required. For non-convulsive status epilepticus, lorazepam may be administered to control evidence of seizures (e.g., 0.5 mg I.V., with increased dosage by 0.5 mg increment to 2 mg I.V. total). For convulsive status epilepticus, 2 mg I.V. lorazepam may be administered to control seizures, with additional 2 mg I.V. as required. Maintenance dosing of levetiracetam, lorazepam and/or phenobarbital, and EEG may be considered for all status epilepticus.

On-Target Off-Tumor Toxicity

In certain embodiments, the methods provided herein further comprise monitoring the development of an on-target off-tumor toxicity resulting from the administration of a first dose of engineered cells (e.g., CAR-T cells; CART-TnMUC1; CART-PSMA-TGFβRDN).

The ideal target antigen is restricted to the tumor cell and provides a critical survival signal for the malignant clone. Many targets of redirected immune cells (e.g., CAR-T cells; CART-TnMUC1; CART-PSMA-TGFβRDN) have shared expression on normal tissues and some degree of on-target off-tumor toxicity occurs through engagement of target antigen on nonpathogenic tissues. The severity of reported events has ranged from manageable lineage depletion (B-cell aplasia) to severe toxicity (death). On-target off-tumor recognition is predictably seen in a variety of organ systems, including gastrointestinal, hematologic, and pulmonary. As used herein, the term “on-target off-tumor toxicity” refers to any toxicity that results in the recognition of a nonpathogenic cell by a redirected immune cell (e.g., a CAR-T cell; CART-TnMUC1; CART-PSMA-TGFβRDN) as employed in adoptive cell therapies. In some embodiments, the on-target off-tumor toxicity is a glandular toxicity. In some embodiments, the on-target off-tumor toxicity is pancreatitis. In some embodiments, the on-target off-tumor toxicity is a gastrointestinal toxicity. On-target off-tumor toxicities may be diagnosed and/or assessed by a physical examination of the subject.

In certain embodiments, where the redirected immune cell is a PSMA targeting CAR-T cell, on-target off-tumor toxicity may include parotiditis and/or neurologic toxicity associated with the expression of PSMA in a normal (e.g., non-diseased) tissue. Parotiditis is inflammation of the parotid salivary gland and can be acute, chronic, or chronic with acute exacerbations. Accordingly, the methods herein further comprise monitoring the development of a glandular toxicity (e.g., parotiditis) and/or neurologic toxicity associated with the expression of PSMA in a normal tissue. In certain embodiments, the normal tissue is a salivary gland and/or the hypothalamus. Methods for monitoring the development of a glandular toxicity is known to those of skill in the art (e.g., a physician or clinician). In some embodiments, methods for monitoring the development of a glandular toxicity (e.g., parotiditis) includes a physical examination of the subject. In some embodiments, the physical examination includes examining the subject for pain or glandular dysfunction (e.g., dry mouth).

In certain embodiments, where the redirected immune cell is a Tn-MUC1 targeting CAR-T cell, on-target off-tumor toxicity may include pancreatitis, renal insufficiency, and/or gastrointestinal inflammation. Accordingly, the methods herein further comprise monitoring the development of pancreatitis, renal insufficiency, and/or gastrointestinal inflammation resulting from the administration of an immune cell comprising a Tn-MUC1-CAR. Methods for monitoring the development of renal insufficiency is known to those of skill in the art (e.g., a physician or clinician). In some embodiments, monitoring the development of renal insufficiency includes assessing the subject for elevated levels of creatinine. In some embodiments, monitoring the development of renal insufficiency includes assessing the subject using kidney function tests that are known in the art. Methods for monitoring the development of pancreatitis is known to those of skill in the art (e.g., a physician, or a clinician). In some embodiments, monitoring the development of pancreatitis includes, e.g., a physical examination. In some embodiments, methods for monitoring the development of pancreatitis by a physical examination include examining the subject for abdominal pain. In some embodiments, methods for monitoring the development of pancreatitis include assessing the blood levels of certain markers associated with pancreatitis, e.g., amylase or lipase. In some embodiments, methods for monitoring the development of pancreatitis include assessing the elevation of blood levels of amylase and lipase.

Accordingly, the present disclosure provides a method of treating a solid tumor in a subject, comprising administering to the subject a first dose of cells, wherein the cells comprise a chimeric antigen receptor (CAR) having affinity for a solid tumor antigen, and wherein the first dose comprises about 30% of a total dose of cells; and administering to the subject a consecutive dose of cells comprising the CAR, wherein the consecutive dose comprises about 70% of the total dose of cells, and wherein the consecutive dose is administered at least five days after the administration of the first dose. In certain embodiments, the present disclosure provides a method of treating a solid tumor in a subject in need thereof, comprising: administering to a subject a first dose of cells, wherein the cells comprise a chimeric antigen receptor (CAR) having affinity for a solid tumor antigen, and wherein the first dose comprises about 30% of a total dose of cells; monitoring the development of cytokine release syndrome, immune cell-associated neurologic toxicities, and/or an on-target off-tumor toxicity resulting from the administration of the first dose; and administering to the subject a consecutive dose of cells comprising the CAR, wherein the consecutive dose comprises about 70% of the total dose of cells, and wherein the consecutive dose is administered at least five days after the administration of the first dose.

In certain exemplary embodiments, the solid tumor is metastatic castrate resistant prostate cancer and the solid tumor antigen is prostate-specific membrane antigen (PSMA). In certain exemplary embodiments, the cells comprising a PSMA-CAR further comprise a dominant negative truncated variant of a TGFβ receptor. In certain exemplary embodiments, the method further comprises monitoring the development of a toxicity selected from the group consisting of cytokine release syndrome, immune cell-associated neurologic toxicities, and an on-target off-tumor toxicity (e.g., parotiditis and a neurologic toxicity associated with the expression of PSMA in a normal tissue, e.g., a salivary gland and/or the hypothalamus). In certain embodiments, monitoring the development of a toxicity occurs after the administration of the first dose of cells (e.g., about 30% of total dose of cells), and is performed by assessing one or more criteria (e.g., biomarkers) as compared to the criteria measured in the subject prior to administration of any cells. In certain embodiments, the consecutive dose (e.g., about 70% of total dose of cells) is administered at a time when the toxicity has been treated and/or has subsided.

Accordingly, the present disclosure provides a method of treating metastatic castrate resistant prostate cancer in a subject in need thereof, comprising: administering to a subject a first dose of cells, wherein the cells comprise a chimeric antigen receptor (CAR) having affinity for prostate-specific membrane antigen (PSMA-CAR), and a truncated variant of TGFβ receptor type II (dnTGFβ R2), and wherein the first dose comprises about 30% of a total dose of cells; monitoring the development of parotiditis, and/or a neurologic toxicity associated with the expression of PSMA in a normal tissue resulting from the administration of the first dose; and administering to the subject a consecutive dose of cells comprising the PSMA-CAR and dnTGFβ R2, wherein the consecutive dose comprises about 70% of the total dose of cells, and wherein the consecutive dose is administered at least five days after the administration of the first dose.

In certain exemplary embodiments, the solid tumor is selected from the group consisting of non-small cell lung cancer, triple negative breast cancer, pancreatic adenocarcinoma, and ovarian and fallopian tube cancer, and the solid tumor antigen is a truncated glycoepitope of mucin-1 (Tn-MUC1). In certain exemplary embodiments, the method further comprises monitoring the development of a toxicity selected from the group consisting of cytokine release syndrome, immune cell-associated neurologic toxicities, and an on-target off-tumor toxicity (e.g., pancreatitis, renal insufficiency and/or gastrointestinal inflammation). In certain embodiments, monitoring the development of a toxicity occurs after the administration of the first dose of cells (e.g., about 30% of total dose of cells), and is performed by assessing one or more criteria (e.g., biomarkers) as compared to the criteria measured in the subject prior to administration of any cells. In certain embodiments, the consecutive dose (e.g., about 70% of total dose of cells) is administered at a time when the toxicity has been treated and/or has subsided.

Accordingly, the present disclosure provides a method of treating a cancer in a subject in need thereof, comprising: administering to a subject a first dose of cells, wherein the cells comprise a chimeric antigen receptor (CAR) having affinity for a truncated glycoepitope of mucin-1 (TnMUC1), and wherein the first dose comprises about 30% of a total dose of cells; monitoring the development of pancreatitis, renal insufficiency, and/or gastrointestinal inflammation resulting from the administration of the first dose; and administering to the subject a consecutive dose of cells comprising the TnMUC1-CAR, wherein the consecutive dose comprises about 70% of the total dose of cells, and wherein the consecutive dose is administered at least five days after the administration of the first dose. In certain embodiments, the cancer is selected from the group consisting of non-small cell lung cancer, triple negative breast cancer, pancreatic adenocarcinoma, and ovarian and fallopian tube cancer.

Patient Selection

Methods provided herein involve selecting and treating a subject suitable for treatment. Accordingly, the present disclosure provides inclusion and exclusion criteria for subjects suitable for treatment using a method described herein.

For CART-PSMA-TGFβRDN therapy, in an exemplary embodiment, a suitable subject must have a confirmed diagnosis of metastatic castrate resistant prostate cancer (mCRPC).

In an exemplary embodiment, a suitable subject must have a testosterone level <50 ng/ml.

In an exemplary embodiment, a suitable subject must have received at least 2 prior lines of therapy for metastatic prostate cancer, including at least one second generation androgen receptor inhibitor (e.g. enzalutamide or apalutamide) and/or CYP17α inhibitor (e.g. abiraterone/prednisone). Androgen deprivation therapy (ADT) with GnRH does not count as a line of therapy.

In an exemplary embodiment, a suitable subject must have evidence of progressive castrate resistant prostate adenocarcinoma, as defined by:

a. Castrate levels of testosterone (<50 ng/ml); and

b. Evidence of one of the following measures of progressive disease in the 12 weeks preceding eligibility confirmation by physician:

i. soft tissue progression by RECIST 1.1 criteria;

ii. osseous disease progression with 2 or more new lesions on bone scan; and/or

iii. increase in serum PSA of at least 25% and an absolute increase of 2 ng/ml or more from nadir on at least three consecutive tests a minimum of 1 week apart.

In an exemplary embodiment, a suitable subject must have adequate vital organ function as defined by:

a. Estimated creatinine clearance ≥30 ml/min by MDRD;

b. ALT and AST ≤3× the upper limit of normal and total bilirubin ≤2.0 mg/dL;

c. Serum total bilirubin <1.5×ULN unless patient has known Gilbert's Syndrome, and no other reason for indirect bilirubinemia;

d. Serum albumin ≥3.0 g/dL; and/or

e. Left ventricular ejection fraction (LVEF) ≥45%. LVEF assessment must have been performed within 8 weeks of enrollment.

In an exemplary embodiment, a suitable subject must have adequate hematologic reserve (without the use of supportive transfusion or hematopoietic growth factors within 4 weeks of apheresis), as defined by:

a. Hemoglobin ≥9 g/dL;

b. Absolute neutrophil count ≥1000/μL; and

c. Platelet count ≥100,000/μL.

In an exemplary embodiment, a suitable subject must not be transfusion-dependent to maintain hematologic parameters.

In an exemplary embodiment, a suitable subject must have PSMA+ disease, determined by centrally tested PSMA IHC expression in an archival tumor biopsy. If an archival tumor biopsy sample is not available, then the patient may undergo a biopsy for the purposes of screening eligibility with only non-significant risk biopsy procedures.

In an exemplary embodiment, a suitable subject who has not undergone bilateral orchiectomy must be able to continue GnRH therapy during the study.

In an exemplary embodiment, a suitable subject must have evaluable disease per Prostate Cancer Working Group (PCWG3) criteria.

In an exemplary embodiment, a suitable subject must have an Eastern Cooperative Oncology Group (ECOG) score of 0 or 1.

In an exemplary embodiment, a suitable subject having toxicities from any previous therapy must have recovered to Grade 1 or baseline.

In an exemplary embodiment, a suitable subject must have life expectancy greater than 3 months.

In an exemplary embodiment, a suitable subject of reproductive potential must agree to use approved contraceptive methods per protocol.

For CART-PSMA-TGFβRDN therapy, in an exemplary embodiment, a suitable subject must not have the following:

1. Active invasive cancer, other than the proposed cancer included in the study (e.g., mCRPC), within 2 years prior to screening, unless treated with curative intent, i.e. nonmelanoma skin cancer.

2. Current treatment with systemic steroids (defined as a dose greater than the equivalent of prednisone 10 mg/day). Low-dose physiologic replacement therapy with corticosteroids equivalent to prednisone 10 mg/day or lower, topical steroids, and inhaled steroids are acceptable.

3. Active autoimmune disease (including connective tissue disease, uveitis, sarcoidosis, inflammatory bowel disease or multiple sclerosis) or a history of severe autoimmune disease requiring prolonged immunosuppressive therapy. Patients should have stopped any immunosuppressive therapy within 6 weeks prior to screening visit.

4. Current, active HIV, HCV, HBV infections. Viral testing at Screening is required in all patients to rule out subclinical infections. Patients who are HBcAb positive are excluded, even if HBSAg negative.

5. Other active or uncontrolled medical or psychiatric condition that would preclude participation.

6. Prior allogeneic stem cell transplant.

7. Active and untreated central nervous system (CNS) malignancy. Treated lesions may be considered inactive if they are stable for at least 1 month following definitive treatment. Patient must not require corticosteroid therapy or anti-epileptic medications for the management of brain metastases.

8. History of severe infusion reaction to monoclonal antibodies or biological therapies, or to study product excipients (e.g. human serum albumin, dimethyl sulfoxide (DMSO), dextran 40) that would preclude the patient safely receiving CART-PSMA-TGFβRDN cells.

9. History of being previously treated with a J591 antibody-based therapy.

10. Active or recent (within the past 6 months prior to apheresis) cardiac disease, defined as (1) New York Heart Association (NYHA) Class III or IV heart failure, (2) unstable angina or (3) a history of recent (within 6 months) myocardial infarction or sustained (>30 second) ventricular tachyarrhythmias.

11. Have inadequate venous access for or contraindications for the apheresis procedure.

For CART-PSMA-TGFβRDN therapy, in another exemplary embodiment, a suitable subject must have a confirmed histologic diagnosis of prostate cancer and have metastatic castrate resistant prostate cancer (mCRPC).

In certain exemplary embodiments, a suitable subject has had prior therapies defined as at least two prior lines of systemic therapy for prostate cancer, including at least one second generation androgen receptor inhibitor and/or CYP17α inhibitor. In certain exemplary embodiments, at least one line of prior therapy must be in the mCRPC setting.

In certain exemplary embodiments, a suitable subject has evidence of disease as defined as castrate levels of testosterone (<50 ng/mL); and evidence of one of the following measures of progressive disease in the twelve weeks preceding eligibility confirmation by a physician: (1) soft tissue progression by Response Evaluation Criteria in Solid Tumors (RECIST) 1.1 criteria; (2) osseous disease progression with two or more new lesions on bone scan; (3) increase in serum PSA of at least 25% and an absolute increase of 2 ng/mL or more from nadir on at least three consecutive tests a minimum of one week apart.

In certain exemplary embodiments, a suitable subject has PSMA+ disease determined by centrally tested PSMA expression in prior or archival tumor sample.

In certain exemplary embodiments, a suitable subject has an evaluable disease per Prostate Working Group 3 (PCWG3) criteria.

In certain exemplary embodiments, a suitable subject has an Eastern Cooperative Oncology Group (ECOG) score of 0 or 1.

In certain exemplary embodiments, a suitable subject has a life expectancy of greater than three months.

In certain exemplary embodiments, a suitable subject with toxicities from any previous therapy must have recovered to Grade 1 or baseline.

In certain exemplary embodiments, a suitable subject who has not undergone bilateral orchiectomy must be able to continue gonadotropin-releasing hormone (GnRH) therapy during the therapy.

In certain exemplary embodiments, a suitable subject has estimated creatinine clearance ≥60 mL/min by Modification of Diet in Renal Disease criteria.

In certain exemplary embodiments, a suitable subject has levels of alanine aminotransferase (ALT) and aspartate aminotransferase (AST)≤2.5× the upper limit of normal (ULN).

In certain exemplary embodiments, a suitable subject with hepatic metastases has levels of ALT and AST ≤3.0×ULN.

In certain exemplary embodiments, a suitable subject has serum total bilirubin <1.5 mg/dL unless the subject has known Gilbert's Syndrome, then a serum total bilirubin of ≤3 mg/dL.

In certain exemplary embodiments, a suitable subject has serum albumin ≥3.0 g/dL.

In certain exemplary embodiments, a suitable subject has a left ventricular ejection fraction (LVEF) ≥50%; wherein LVEF assessment must have been performed within eight weeks of start of therapy.

In certain exemplary embodiments, a suitable subject has a hemoglobin level ≥9 g/dL.

In certain exemplary embodiments, a suitable subject has an absolute neutrophil count ≥1,500/uL.

In certain exemplary embodiments, a suitable subject has a platelet count ≥100,000/uL.

In certain exemplary embodiments, a suitable subject of reproductive potential agrees to use of approved highly effective contraceptive methods.

In certain exemplary embodiments, a suitable subject must agree not to participate in a conception process or must agree to a highly effective method of contraceptive.

In certain exemplary embodiments, a suitable subject must not have active invasive cancer, other than the cancer intended for therapy (e.g., mCRPC), within two years prior to screening, unless treated with curative intent.

In certain exemplary embodiments, a suitable subject must not have current treatment with corticosteroids (defined as a dose greater than the equivalent of prednisone 10 mg/day).

In certain exemplary embodiments, a suitable subject must not have active autoimmune disease (including connective tissue disease, uveitis, sarcoidosis, inflammatory bowel disease or multiple sclerosis) or a history of severe autoimmune disease requiring prolonged immunosuppressive therapy (any immunosuppressive therapy within six weeks prior to screening visit).

In certain exemplary embodiments, a suitable subject must not have current, active human immunodeficiency virus (HIV), hepatitis C (HCV), hepatitis B virus (HBV) infections.

In certain exemplary embodiments, a suitable subject must not have had prior allogeneic stem cell transplant.

In certain exemplary embodiments, a suitable subject must not have active and untreated central nervous system (CNS) malignancy.

In certain exemplary embodiments, a suitable subject must not have a history of severe infusion reaction to monoclonal antibodies or biological therapies, or to therapy product excipients that would preclude the subject safely receiving CART-PSMA-TGFβRDN cells.

In certain exemplary embodiments, a suitable subject must not have a history of being previously treated with a J591 antibody-based therapy.

In certain exemplary embodiments, a suitable subject must not have active or recent (within the past 6 months prior to apheresis) cardiac disease, defined as (1) New York Heart Association (NYHA) Class III or IV heart failure, (2) unstable angina or (3) a history of recent (within 6 months) myocardial infarction or sustained (>30 second) ventricular tachyarrhythmias.

In certain exemplary embodiments, a suitable subject must not have inadequate venous access for or contraindications for the apheresis procedure.

For CART-TnMUC1 therapy, in an exemplary embodiment, a suitable subject must have a confirmed diagnosis of metastatic treatment-resistant ovarian cancer (including cancers of the fallopian tube), pancreatic adenocarcinoma, hormone receptor (HR)-negative and HER2-negative (triple negative) breast cancer (TNBC) or non-small cell lung cancer (NSCLC), or relapsed/refractory multiple myeloma.

In some embodiments, a suitable subject has an ECOG score of 0 or 1.

In some embodiments, a suitable subject has received prior therapy for multiple myeloma: relapsed or refractory disease after either one of the following (i) at least 3 prior regimens, which must have contained an alkylating agent, proteasome inhibitor, and thalidomide analog (lenalidomide or pomalidomide), (ii) at least 2 prior regimens if ‘double-refractory’ to a proteasome inhibitor and thalidomide analog, defined as progression on or within 60 days of treatment with these agents, and/or (iii) patients must be at least 90 days since autologous stem cell transplant (ASCT), if performed.

In some embodiments, induction therapy, autologous stem cell transplant (ASCT), and maintenance therapy if given sequentially without intervening progression are considered 1 ‘regimen.’

In some embodiments, a suitable subject has received prior therapy for non-small cell lung cancer (NSCLC). In one embodiment, a suitable subject having had prior therapy for NSCLC has received standard therapy, including both checkpoint inhibition (PD-1/PD-L1 directed therapy) and platinum-based chemotherapy or be intolerant of these standard therapies. In one embodiment, a suitable subject having had prior therapy for NSCLC with EGFR or ALK alterations has received prior targeted therapy directed at the specific identified mutations in addition to the standard therapy classes described above.

In some embodiments, a suitable subject has received prior therapy for pancreatic adenocarcinoma. In one embodiment, a suitable subject having had prior therapy for pancreatic adenocarcinoma has experienced disease progression following at least one standard of care systemic chemotherapy for metastatic or unresectable disease.

In some embodiments, a suitable subject has received prior therapy for triple-negative breast cancer (TNBC). In one embodiment, a suitable subject having had prior therapy for TNBC has experienced disease progression following at least one prior systemic anti-cancer therapy regimen as part of their treatment for management of metastatic breast cancer.

In some embodiments, a suitable subject has received prior therapy for ovarian cancer. In one embodiment, a suitable subject having had prior therapy for ovarian cancer is suitable if considered platinum-resistant (initially sensitive to platinum therapy) and has received at least two prior lines of therapy for metastatic ovarian cancer, including at least one prior line of therapy including a platinum-containing regimen.

In some embodiments, a suitable subject has an evaluable disease.

In one embodiment, a suitable subject having multiple myeloma is suitable if: the subject has measurable disease on treatment (study) entry, which includes at least one of the following: (1) Serum M spike ≥0.5 g/dL; (2) 24-hour urine M-spike ≥200 mg; (3) Involved serum free light chain (FLC) ≥50 mg/L with abnormal ratio; (4) Measurable plasmacytoma on examination or imaging; (5) Bone marrow plasma cells ≥20%.

In some embodiments, subjects with IgA myeloma in whom serum protein electrophoresis is deemed unreliable, due to co-migration of normal serum proteins with the paraprotein in the beta region, may be suitable as long as total serum IgA level is elevated above normal range.

In one embodiment, a suitable subject having a solid tumor will have their disease status assessed as per Response Evaluation Criteria In Solid Tumors Criteria (RECIST v.1.1; see, Eisenhauer et al. (2009) Eur J Cancer, 45(2):228-247). Tumor imaging may be performed at least within 28 days before apheresis. Phase-specific criteria include: Phase 1: subjects must have evaluable disease in Phase 1 per RECIST v.1.1; Phase 1a expansion: subjects must have measurable disease in Phase 1a expansion per RECISTv.1.1.

In some embodiments, suitable subjects have a TnMUC1+ disease, determined by centrally tested TnMUC1 expression in a prior or archival tumor biopsy. If an archival tumor biopsy sample is not available, then the subject may undergo an optional biopsy for the purposes of screening eligibility with only non-significant risk biopsy procedures.

In some embodiments, suitable subjects have completed prior anti-cancer therapy at least 2 weeks prior to Screening and toxicities from any previous therapy must have recovered to grade 1 or 0 (with the exception of alopecia, well controlled electrolyte or endocrine abnormalities, well-controlled peripheral neuropathy, and vitiligo).

In some embodiments, suitable subjects have a life expectancy greater than 3 months.

In some embodiments, suitable subjects have adequate vital organ function as defined by:

(1) Serum creatinine <1.5 mg/dL or estimated creatinine clearance ≥30 ml/min (per Institutional standard calculation);

(2) Alanine aminotransferase (ALT) and aspartate aminotransferase (AST)≤3× the upper limit of normal (ULN) and total bilirubin ≤2.0 mg/dL. No specific exclusions are made for patients with hepatic disease;

(3) Serum total bilirubin <1.5×ULN;

(4) Serum albumin ≥3.0 g/dL (solid tumor patients in Arm 1 and Phase 1a only, not applicable to patients with multiple myeloma);

(5) Left ventricular ejection fraction (LVEF) ≥45%. LVEF assessment must have been performed within 8 weeks of screening.

In some embodiments, suitable subjects have adequate hematologic reserve (without the use of supportive transfusion or hematopoietic growth factors within 4 weeks of apheresis), as defined by:

(1) Hemoglobin ≥9 g/dL;

(2) Absolute neutrophil count ≥1000/μL;

(3) Platelet count ≥50,000/μL (≥30,000/μL if bone marrow plasma cells are ≥50% of cellularity for myeloma patients);

(4) Absolute lymphocyte count of >500/μL. In one embodiment, suitable subjects must not be transfusion-dependent to maintain hematologic parameters.

In some embodiments, suitable subjects of reproductive potential agree to use approved contraceptive methods per protocol.

In some embodiments, suitable subjects considered for treatment using a method described herein must not meet any of the following criteria:

(1) Active invasive cancer other than the proposed cancers included in the treatment (study);

(2) Current treatment with systemic high-dose corticosteroids (defined as a dose greater than the equivalent of prednisone 20 mg/day). Subjects with multiple myeloma at the time of treatment (study) entry must complete prior active high-dose corticosteroid therapy prior to apheresis and be maintained on low-dose corticosteroid therapy or no corticosteroid therapy. Low-dose physiologic replacement therapy with corticosteroids equivalent to prednisone 20 mg/day or lower is acceptable;

(3) Active autoimmune disease (including connective tissue disease, uveitis, sarcoidosis, inflammatory bowel disease or multiple sclerosis) or have a history of severe autoimmune disease requiring prolonged immunosuppressive therapy (any immunosuppressive therapy should have been stopped within 6 weeks prior to screening visit);

(4) Current, active HIV, HCV, HBV infections. Viral testing at Screening is required in all subjects to rule out subclinical infections;

(5) Other active or uncontrolled medical or psychiatric condition that would preclude participation the treatment regimen;

(6) Prior allogeneic stem cell transplant;

(7) Active and untreated central nervous system (CNS) malignancy. Treated lesions may be considered inactive if they are stable for at least 1 month following definitive treatment. Subject must not require corticosteroid therapy or anti-epileptic medications for the management of brain metastases;

(8) History of severe infusion reaction to monoclonal antibodies or biological therapies, or to study product excipients (e.g., human serum albumin, DMSO, dextran 40) that would preclude the patient safely receiving CART-TnMUC1 cells;

(9) Active or recent (within the past 6 months prior to apheresis) cardiac disease, defined as (i) New York Heart Association (NYHA) Class III or IV heart failure, (ii) unstable angina or (iii) a history of recent (within 6 months) myocardial infarction or sustained (>30 second) ventricular tachyarrhythmias;

(10) Have inadequate venous access for or contraindications for the apheresis procedure;

(11) Pregnant or breastfeeding women.

For CART-TnMUC1 therapy, in another exemplary embodiment, a suitable subject must have a confirmed diagnosis of metastatic treatment-resistant ovarian cancer (including cancers of the fallopian tube), pancreatic adenocarcinoma, hormone receptor (HR)-negative and HER2-negative (triple negative) breast cancer (TNBC) or non-small cell lung cancer (NSCLC), or relapsed/refractory multiple myeloma.

In some embodiments, a suitable subject has an Eastern Cooperative Oncology Group (ECOG) score of 0 or 1.

In some embodiments, a suitable subject has had prior therapies as defined by tumor type, as described herein.

In some embodiments, a suitable subject has an evaluable disease as defined by tumor type, as described herein.

In some embodiments, a suitable subject has TnMUC1+ disease, determined by centrally tested TnMUC1 expression in a prior or archival tumor biopsy.

In some embodiments, a suitable subject has completed prior anti-cancer therapy at least 2 weeks prior to Screening and toxicities.

In some embodiments, a suitable subject has a life expectancy greater than 3 months.

In some embodiments, a suitable subject has a level of serum creatinine ≤1.2 mg/dL or calculated creatinine clearance ≥60 ml/min (using the Cockroft & Gault formula).

In some embodiments, a suitable subject has a level of asparatate aminotransferase (AST) or alinine aminotransferase (ALT)≤2.5× upper institutional limit of normal with the following exception: Patients with known hepatic metastases, AST or ALT ≤3× upper institutional limit of normal.

In some embodiments, a suitable subject has a level of serum total bilirubin <1.5 mg/dL with the following exception: patients with known Gilbert's disease, serum total bilirubin <3 mg/dL.

In some embodiments, a suitable subject has a level of serum albumin ≥3.0 g/dL (solid tumor patients in Arm 1 and Phase 1a only, not applicable to patients with multiple myeloma).

In some embodiments, a suitable subject has been assessed with left ventricular ejection fraction (LVEF) ≥50%. LVEF assessment must have been performed within 8 weeks of screening.

In some embodiments, a suitable subject has a level of hemoglobin ≥9 g/dL.

In some embodiments, a suitable subject has a level of absolute neutrophil count ≥1500/μL.

In some embodiments, a suitable subject has a level of platelet count ≥100,000/μL (≥30,000/μL if bone marrow plasma cells are ≥50% of cellularity for myeloma patients).

In some embodiments, a suitable subject has a level of absolute lymphocyte count of >500/μL.

In some embodiments, suitable subjects considered for treatment using a method described herein must not have or be any of the following:

(1) Active invasive cancer other than the proposed cancers included in the study;

(2) Current treatment with systemic high-dose corticosteroids (defined as a dose greater than the equivalent of prednisone 20 mg/day);

(3) Active autoimmune disease (including connective tissue disease, uveitis, sarcoidosis, inflammatory bowel disease or multiple sclerosis) or have a history of severe autoimmune disease requiring prolonged immunosuppressive therapy (any immunosuppressive therapy should have been stopped within 6 weeks prior to screening visit);

(4) Current, active human immunodeficiency virus (HIV), hepatitis C virus (HCV), hepatitis B virus (HBV) infections;

(5) Prior allogeneic stem cell transplant;

(6) Active and untreated central nervous system (CNS) malignancy;

(7) History of severe infusion reaction to monoclonal antibodies or biological therapies, or to study product excipients (eg, human serum albumin, dimethyl sulfoxide [DMSO], dextran 40) that would preclude the patient safely receiving CART-TnMUC1 cells;

(8) Active or recent (within the past 6 months prior to apheresis) cardiac disease, defined as (1) New York Heart Association (NYHA) Class III or IV heart failure, (2) unstable angina or (3) a history of recent (within 6 months) myocardial infarction or sustained (>30 second) ventricular tachyarrhythmias;

(9) Have inadequate venous access for or contraindications for the apheresis procedure; and/or

(10) Pregnant or breastfeeding women.

### H. Articles of Manufacture

The present disclosure also provides articles of manufacture, such as devices and kits, for the administration of engineered cells to subjects. The articles of manufacture allow for the administration of engineered cells to subjects in accordance to the methods provided herein and known in the art.

The articles of manufacture include one or more containers, typically a plurality of containers, packaging material, and a label or package insert on or associated with the container or containers and/or packaging, generally including instructions for administration of the cells to a subject.

The containers generally contain the cells to be administered (e.g., CART-TnMUC1; CART-PSMA-TGFβRDN), e.g., one or more unit doses thereof. The article of manufacture typically includes a plurality of containers, each containing a single unit dose of the cells. The unit dose may be about 30% of the total dose of cells to be administered to the subject in the first dose or about 70% of the total dose of cells to be administered in a consecutive dose.

Suitable containers include, for example, flexible bags, such as infusion bags, vials, bottles, and syringes. In some embodiments, the containers are bags, e.g., flexible bags, such as those suitable for infusion of cells to subjects, e.g., PVC or flexible plastic bags, and/or IV solution bags. The bags in some embodiments are sealable and/or able to be sterilized, so as to provide sterile solution and delivery of the cells and compositions. In some embodiments, the containers have a capacity of from about 10 ml to about 1000 ml capacity, such as from about 10 ml to about 100, or from about 10 ml to about 500 ml capacity. In some embodiments, the containers, e.g., bags, are and/or are made from material which is stable and/or provide stable storage and/or maintenance of cells at one or more of various temperatures, such as in cold temperatures, e.g. below at or about or at or about −20° C., −80° C., −120° C., 135° C. and/or temperatures suitable for cryopreservation, and/or other temperatures, such as temperatures suitable for thawing the cells and body temperature such as at or about 37° C., for example, to permit thawing, e.g., at the subject's location or location of treatment, e.g., at bedside, immediately prior to treatment.

The containers may be formed from a variety of materials such as glass or plastic. In some embodiments, the container has one or more port, e.g., sterile access ports, for example, for connection of tubing or cannulation to one or more tubes, e.g., for intravenous or other infusion and/or for connection for purposes of transfer to and from other containers, such as cell culture and/or storage bags or other containers. Exemplary containers include infusion bags, intravenous solution bags, vials, including those with stoppers pierceable by a needle for injection. The choice of variety of material will be made by those of skill in the art such that the contains can be kept sterile, and/or sterilized.

The article of manufacture may further include a package insert or label with one or more pieces of identifying information and/or instructions for use. In some embodiments, the information or instructions indicates that the contents can or should be used to treat a particular condition or disease, and/or providing instructions therefor. The label or package insert may indicate that the contents of the article of manufacture are to be used for treating the disease or condition. In some embodiments, the label or package insert provides instructions to treat a subject, e.g., the subject from which the cells have been derived, via a method involving the administration of a first and one or more consecutive doses of the cells, e.g., according to any of the embodiments of the provided methods. In some embodiments, the instructions specify administration, in a first dose, of one unit dose, e.g., the contents of a single individual container in the article of manufacture, followed by a consecutive dose at a specified time point or within a specified time window and/or after the detection of the presence or absence or amount or degree of one or more factors or outcomes in the subject. In some embodiments, the instructions specify administering a first administration and a consecutive administration. In some embodiments, the first administration comprises delivering one of said unit doses to the subject and the consecutive administration comprises administering a second unit dose to the subject. In some embodiments, the instructions specify that the consecutive administration is to be carried out at a time between about 5 and about 7 days following the first administration, e.g., following the initiation of the first administration. In some embodiments, the instructions specify that the consecutive dose is to be administered at a time after which it has been determined that development of cytokine release syndrome, immune cell-associated neurologic toxicities, and/or an on-target off-tumor toxicity has been adequately managed.

In some embodiments, the label or package insert or packaging comprises an identifier to indicate the specific identity of the subject from which the cells are derived and/or are to be administered. In the case of autologous transfer, the identity of the subject from which the cells are derived is the same as the identity of the subject to which the cells are to be administered. Thus, the identifying information may specify that the cells are to be administered to a particular patient, such as the one from which the cells were originally derived. Such information may be present in the packaging material and/or label in the form of a bar code or other coded identifier, or may indication the name and/or other identifying characteristics of the subject.

The article of manufacture in some embodiments includes one or more, typically a plurality, of containers containing compositions comprising the cells, e.g., individual unit dose forms thereof, and further include one or more additional containers with a composition contained therein which includes a further agent, such as a cytotoxic or otherwise therapeutic agent, for example, which is to be administered in combination, e.g., simultaneously or sequentially in any order, with the cells. Alternatively, or additionally, the article of manufacture may further include another or the same container comprising a pharmaceutically-acceptable buffer. It may further include other materials such as other buffers, diluents, filters, tubing, needles, and/or syringes.

The term “package insert” as used herein, refers to instructions typically included in commercial packages of therapeutic products, that contain information about the dosage, indication, administration, contraindications, combination therapy, and/or warnings concerning the use of such therapeutic products.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### I. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” or “approximately” as used herein when referring to a measurable value such as an amount, a temporal duration, and the like, is meant to encompass variations of 20% or +10%, +5%, 1%, or 0.1% of a given value or range, as such variations are appropriate to perform the disclosed methods.

As used herein, “administer” or “administration” refers to the act of injecting or otherwise physically delivering a substance as it exists outside the body (e.g., a CAR-T composition) into a patient, such as by, but not limited to, pulmonary (e.g., inhalation), mucosal (e.g., intranasal), intradermal, intravenous, intratumoral, intramuscular delivery and/or any other method of physical delivery described herein or known in the art. In certain embodiments, the substance is delivered systemically. In certain exemplary embodiments, the substance is delivered intravenously. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms. In some embodiments, the substance may be delivered after the disease has been treated and the disease is refractory to the initial treatment. In some embodiments, the substance may be delivered after the disease has been treated and is in remission. In such embodiments, the substance is delivered to prevent and/or treat the onset of a relapse in disease.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

“Allogeneic” refers to any material derived from a different animal of the same species.

As used herein, the term “antibody” refers to such assemblies (e.g., intact antibody molecules, immunoadhesins, or variants thereof) which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

As will be discussed in more detail below, the generic term “antibody” comprises five distinct classes of antibody that can be distinguished biochemically. There are five classes of antibodies, each of which are clearly within the scope of the present disclosure. Focusing the discussion on the IgG class of immunoglobulins, immunoglobulins comprise two identical heavy chains of molecular weight 53,000-70,000, and two identical light chains of molecular weight approximately 23,000 Daltons, joined together by disulfide bonds in a “Y” configuration. The “Y” configuration is made up of the light chains that bracket the heavy chains, where the heavy chains start at the top of the “Y” and continue through the variable region.

Each class of heavy chain may be bound by a light chain. The immunoglobulin light chains are classified as either lambda (k) or kappa (x). Generally, the heavy and light chains are bound together via covalent linkages, and the “tail” portions of the two heavy chains are bound together via disulfide covalent linkages or non-covalent linkages when the immunoglobulins are generated either by genetically engineered host cells, B cells, or hybridomas. The ordinarily skilled person appreciates that heavy chains are classified as alpha (α), delta (δ), epsilon (ε), gamma (γ), or mu (μ), with subclassifications among each (e.g., γ1-γ4). Heavy chain amino acid sequences start at the forked ends of the “Y” configuration (N-terminus) to the bottom of each chain (C-terminus). The nature of the heavy chain determines the antibody class, e.g., IgA, IgE, IgM, or IgG. Immunoglobulin isotype subclasses (e.g., IgG1-G4, IgA1, etc.) are well-characterized and are known to confer functional specialization. The skilled artisan would readily be able to discern modified versions of each of these classes and, accordingly, are within the scope of the present disclosure.

Both the light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarity determining regions” or “CDRs” interspersed among “framework regions,” as defined herein.

As used herein, the term “VH domain” or “VH region” includes the amino terminal variable domain (or region) of an immunoglobulin heavy chain, and the term “VL domain” or “VL region” includes the amino terminal variable domain (or region) of an immunoglobulin light chain.

As indicated above, the variable regions of an antibody confer specificity to the antibody for binding epitopes on antigens. The VH and VL domains of an antibody combine to form the variable region (Fv) that defines a three-dimensional antigen binding site. This quaternary antibody structure forms the antigen binding site present at the end of each arm of the “Y” configuration. The antigen binding site is defined by three CDRs on each of the heavy and light chain variable regions. As used herein, the term “antigen binding site” or “antigen binding domain” includes a site that specifically binds (immunoreacts with) an antigen (e.g., a cell surface or soluble antigen). The antigen binding site may include an immunoglobulin heavy and light chain variable regions, and the formation of the antigen binding site by these variable regions determines antibody specificity. An antigen binding site is formed by variable regions that vary from one antibody to another.

In certain embodiments, antibodies or antigen binding fragments thereof of the present disclosure comprise at least two antigen binding domains that provide for the association of the binding fragment with the selected antigen. The antigen binding domains need not be derived from the same immunoglobulin molecule. In this regard, the variable region may or be derived from any type of animal that can be induced to mount a humoral response and generate immunoglobulins against the desired antigen. As such, the variable region of a binding polypeptide may be, for example, of mammalian origin e.g., may be human, murine, rat, goat, sheep, non-human primate (such as cynomolgus monkeys, macaques, etc.), equine, or camelid (e.g., from camels, llamas and related species).

The six CDRs present on a monomeric antibody are short, non-contiguous sequences of amino acids specifically positioned to form the antigen binding site of the three-dimensional configuration of the antibody in an aqueous environment. The remaining sequences of the heavy and light variable domains show less variability in amino acid sequence between binding polypeptides, and are known as framework regions. Generally, the framework regions have a β-sheet conformation and the CDRs form loops which connect the β-sheet structure. In some cases, the CDRs form loops that are a part of the β-sheet structure. Thus, framework regions act as a scaffold that positions the six CDRs in the proper orientation by inter-chain, non-covalent interactions. The antigen binding domain formed by the correctly positioned CDRs promotes the non-covalent binding of the antibody to an epitope of an immunoreactive antigen.

The antigen binding domain of, e.g., a chimeric antigen receptor, includes antibody variants. As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bi-specific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

As used herein the term “valency” refers to the number of potential target binding sites in a polypeptide. Each target binding site specifically binds one target molecule or specific site on a target molecule. When a polypeptide comprises more than one target binding site, each target binding site may specifically bind the same or different molecules (e.g., may bind to different ligands or different antigens, or different epitopes on the same antigen). The subject binding polypeptides typically has at least one binding site specific for a human antigen molecule.

By the term “synthetic antibody” as used herein, is meant an antibody which is generated using recombinant DNA technology, such as, for example, an antibody expressed by a bacteriophage. The term is also referred to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit a desired immune response. Moreover, the skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it may later to be re-introduced into the individual.

The term “chimeric antigen receptor” or “CAR,” as used herein, refers to an artificial T cell receptor that is engineered to be expressed on an immune effector cell or precursor cell thereof and specifically bind an antigen CARs may be used in adoptive cell therapy with adoptive cell transfer. In some embodiments, adoptive cell transfer (or therapy) comprises removal of T cells from a patient, and modifying the T cells to express the receptors specific to a particular antigen. In some embodiments, the CAR has specificity to a selected target, for example, PSMA, or MUC1. CARs may also comprise an intracellular activation domain, a transmembrane domain and an extracellular domain comprising an antigen binding region.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends.

As used herein, the term “composition” is intended to encompass a product containing the specified ingredients (e.g., a CAR-T provided herein) in, optionally, the specified amounts, as well as any product which results, directly or indirectly, from combination of the specified ingredients in, optionally, the specified amounts.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Donor antigen” refers to an antigen expressed by the donor tissue to be transplanted into the recipient.

“Recipient antigen” refers to a target for the immune response to the donor antigen.

The term “downregulation” as used herein refers to the decrease or elimination of gene expression of one or more genes.

“Effective amount” or “therapeutically effective amount” as used interchangeably herein, refer to an amount of a compound, formulation, material, pharmaceutical agent, or composition, as described herein effective to achieve a desired physiological, therapeutic, or prophylactic outcome in a subject in need thereof. Such results may include, but are not limited to an amount that when administered to a mammal, causes a detectable level of immune response compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like. The effective amount may vary among subjects depending on the health and physical condition of the subject to be treated, the taxonomic group of the subjects to be treated, the formulation of the composition, assessment of the subject's medical condition, and other relevant factors.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. In certain exemplary embodiments, the epitope is about 4-18 amino acids, about 5-16 amino acids, about 6-14 amino acids, about 7-12 amino acids, about 10-12 amino acids, or about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin may be replaced by corresponding non-human residues. Furthermore, humanized antibodies can comprise residues which are found neither in the recipient antibody nor in the imported CDR or framework sequences. Such modifications may be made to refine antibody performance, e.g., optimize specificity, affinity, and capacity. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. See, e.g., Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

The term “immunostimulatory” is used herein to refer to increasing overall immune response.

The term “immunosuppressive” is used herein to refer to reducing overall immune response.

As used herein, “instructional material” refers to a medium of expression that may be used to communicate the utility of a composition and method of the present disclosure. The medium of expression may be in the form of, e.g., a diagram, a recording (e.g., audio or video recording), or a publication. The instructional material may be included in a kit of the present disclosure, and may for example, be included in or attached to a container which contains the composition, or may be transported together with a container that contains the composition. In some embodiments, the instructional material may be transported separately with the intention that the recipient will use the instructional material in conjunction with the container containing the composition.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, e.g., a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “specificity” refers to the ability to specifically bind (e.g., immunoreact with) a given target antigen (e.g., a human target antigen). A chimeric antigen receptor may be monospecific and contain one or more binding sites which specifically bind a target or a chimeric antigen receptor may be multi-specific and contain two or more binding sites which specifically bind the same or different targets. In certain embodiments, a chimeric antigen receptor is specific for two different (e.g., non-overlapping) portions of the same target. In certain embodiments, a chimeric antigen receptor is specific for more than one target.

By the term “specifically binds,” as used herein with respect to an antibody, is meant an antibody or binding fragment thereof (e.g., scFv) which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, a chimeric antigen receptor, or a peptide with a second chemical species, to mean that the interaction is dependent upon the presence of a particular structure (e.g., an antigenic determinant or epitope) on the chemical species; for example, a chimeric antigen receptor recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, the terms “subject” and “patient” are used interchangeably. As used herein, a subject is can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets. Any living organism in which an immune response can be elicited may be a subject or patient. In certain exemplary embodiments, a subject is a human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (p) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

As used herein, the term “therapy” refers to any protocol, method and/or agent (e.g., a CAR-T) that can be used in the prevention, management, treatment and/or amelioration of a disease or a symptom related thereto. In some embodiments, the terms “therapies” and “therapy” refer to a biological therapy (e.g., adoptive cell therapy), supportive therapy (e.g., lymphodepleting therapy), and/or other therapies useful in the prevention, management, treatment and/or amelioration of a disease or a symptom related thereto, known to one of skill in the art such as medical personnel.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the terms “treat,” “treatment” and “treating” refer to the reduction or amelioration of the progression, severity, frequency and/or duration of a disease or a symptom related thereto, resulting from the administration of one or more therapies (including, but not limited to, a CAR-T therapy directed to the treatment of solid tumors). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai virus vectors, adenovirus vectors, adeno-associated virus vectors, retrovirus vectors, lentivirus vectors, and the like.

“Xenogeneic” refers to any material derived from an animal of a different species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLE

The invention is further described by reference to the following examples, which are provided for illustration only. The invention is not limited to the examples, but rather includes all variations that are evident from the teachings provided herein. All publicly available documents referenced herein, including but not limited to U.S. patents and U.S. patent publications, are specifically incorporated by reference.

### Example 1: Phase 1 Study of CART-PSMA-TGFbRDN in Patients with Metastatic Castrate Resistant Prostate Cancer

Methodology:

A multi-center, open-label, Phase 1 study of the safety, tolerability and feasibility of dosing patients harboring metastatic castrate resistant prostate cancer (mCRPC) with genetically modified autologous T cells (CART-PSMA-TGFβRDN cells) engineered to express a chimeric antigen receptor (CAR) capable of recognizing the tumor antigen, PSMA and activating the T cell, is performed.

This is a Phase 1 single-arm trial designed to identify the dose and regimen of CART-PSMA-TGFβRDN cells that can be safely administered intravenously following the lymphodepletion regimen to patients with mCRPC. It uses a modified 3+3 Dose Escalation design to determine the RP2D.

Duration of Study:

The period of patient accrual in the study is anticipated to last 12-16 months. Individual patients are expected to participate for approximately 24 months for treatment, and long-term safety follow-up assessments. Patients will be followed for longer time periods for the final safety and overall survival (OS) analyses.

Study Rationale:

CART-PSMA-TGFβRDN Rationale:

The tumor antigen targeted by CART-PSMA-TGFβRDN is the prostate specific membrane antigen (PSMA). Expression of PSMA on the membrane surface, as well as in the cytoplasm, is observed by immunohistochemistry in most prostate tumors and confers a negative prognosis across patients. This targeting approach is also combined with a dominant negative TGFβ receptor with the goal of modulating the tumor microenvironment and defining the tumor milieu within which the CART-PSMA-TGFβRDN can survive.

The TGFβ/TGFβRI/RII signaling axis is critical to the suppressive function of the myeloid and lymphoid compartments of the immune system, leading to the hypothesis that this signaling axis is critical for the immunosuppressive cells within the tumor microenvironment (TME). With this link, the CART in this program will be armored with the dominant negative TGFβ receptor (TGFβRDN); this receptor lacks the intracellular domain necessary for downstream signaling and prevents the inhibition of the PSMA targeted CART within the TME.

Nonclinical pharmacology data with both in vitro and in vivo models of cancer demonstrate the potential anti-tumor activity that may be achieved in patients with CART-PSMA-TGFβRDN infusion. In addition, nonclinical toxicology data suggest limited to no reactivity is anticipated with normal human tissues. Nonclinical data are described further in the study protocol. Those target tissues deemed as potential risks based on the compilation of non-clinical data are detailed in the study protocol with monitoring and management guidance.

CART-PSMA-TGFβRDN Study Rationale:

This is a Phase 1 dose escalation study using the modified 3+3 design. The study is designed to first identify the optimal dose and regimen (recommended phase 2 dose, RP2D) of the CART-PSMA-TGFβRDN that can be safely administered following the lymphodepletion regimen in patients with mCRPC. The starting dose of CART-PSMA-TGFβRDN is based on prior experience with the CART program in an ongoing First in Human (FIH) study. In the FIH study, two key observations were made: (1) even in the absence of lymphodepletion, the CART-PSMA-TGFβRDN cells demonstrated minor degrees of expansion at the higher dose level tested (1-3×10⁸/m² equivalent to 5-7×10⁸ transduced cells as a flat dose); and (2) more severe toxicity of Grade 4 cytokine release syndrome (CRS) accompanied by neurotoxicity was observed at this dose level when administered following the standard lymphodepletion regimen (fludarabine and cyclophosphamide) in 1/1 patients in cohort 3.

Based on these two key observations, the goal of this Phase 1 study is to explore lower doses of the CART-PSMA-TGFβRDN cells to identify the optimal dose that can be administered as a fractionated dose and lower doses of CART-PSMA-TGFβRDN that could be suitable for additional study.

Study Design:

Study Overview:

The CART-PSMA-TGFβRDN study is an open-label, multi-center Phase 1 study to assess the safety, tolerability, feasibility and preliminary efficacy of the administration of genetically modified autologous T cells engineered to express a CAR capable of recognizing the tumor antigen, PSMA and a dominant negative TGFβ receptor. This study is a Phase 1 study that aims to explore dose ranges and schedules of the CART-PSMATGFβRDN cells in patients with mCRPC.

Overall Patient Pathway:

Following Screening, patients will undergo apheresis, followed by the period of manufacturing of the CART-PSMA-TGFβRDN product (approximately 4-6 weeks). Patients in all cohorts will receive the lymphodepletion (LD) chemotherapy at Day −6 to −4 (with an LD chemotherapy window of −1 day) and CART-PSMA-TGFβRDN cell infusion on Day 0. Follow-up visits will commence per protocol following the CART infusion. After disease progression, patients will continue to be followed for long-term safety assessments and finally for the survival endpoint.

Phase 1 Design:

This study will be a dose escalation study designed to identify the maximum tolerated dose (MTD) and/or the RP2D of CART-PSMA-TGFβRDN cells that can be safely administered to patients in combination with the lymphodepletion regimen of fludarabine and cyclophosphamide in patients with mCRPC. Dose levels to be tested are described below in FIG. 2 and Table 2.

TABLE 2
Dose Levels CART-PSMA-TGFβRDN Study

| Dose | CART-PSMA- | | | |
|---|---|---|---|---|
| Level | TGFβRDN Dose | Regimen | Lymphodepletion | Enrollment |
| 1 | 1-2 × 10⁷ tr cells | Single dose | Days −6 to −4 | 3-6 |
| 2 | 5-6 × 10⁷ tr cells | Single dose | Days −6 to −4 | 4-6 |
| 3 | 1-2 × 10⁸ tr cells | Fractionated dose¹ | Days −6 to −4 | 4-6 |
| 4 | 5-6 × 10⁸ tr cells | Fractionated dose¹ | Days −6 to −4 | 4-6 |
| 5 | 5-6 × 10⁸ tr cells | Single dose | Days −6 to −4 | 4-6 |
| 4a² | 1-2 × 10⁸ tr cells | Single dose | Days −6 to −4 | 4-6 |

tr cells = transduced cells dose
¹Fractionated dose: 30% total dose administered on Day 0 and additional 70% of total dose administered on Day 5-7 based on observed toxicity following Day 0 dose.
²Cohort 4a will open if Cohort 4 is found to be the maximum tolerated dose (MTD), i.e., Cohort 5 does not pass dose limiting toxicity (DLT) criteria.

The DLT observation period for each individual patient is 28 days following the dose of CART cells (administered on Study Day 0). Decisions to escalate, de-escalate or expand an individual dose level cohort will be taken with consideration of all available safety, tolerability and feasibility data for all enrolled cohorts. Patients will be enrolled serially during Phase 1 into an available cohort. All patients will be staggered by at least 7 days from the last day of infusion (i.e. for patients receiving fractionated dosing, the next patient will start 7 days after the last infusion of CART-PSMA-TGFβ DN cells).

Phase 1 Dose Escalation Guidelines:

Escalation or de-escalation will adhere to the following:
A. If 0 DLT of the 3 evaluable patients or 1 DLT out of 6 evaluable patients occurs, the decision can be taken to escalate the dose to the next dose level cohort.
B. If 1 DLT occurs among 3 evaluable patients (1 DLT among 3 patients), then an additional 3 evaluable patients will be enrolled at this dose level. After the cohort is expanded, if 2 DLT occur among the 6 evaluable patients, then the decision should be taken to de-escalate to the prior dose level.
C. If 2 DLT occur among 3 patients, then enrollment in this cohort will be stopped and the dose will be de-escalated to the prior tolerated dose level.
D. Following identification of a potential RP2D, an additional cohort of 3-6 patients can be enrolled in one or both Arms of Phase 1 to confirm the DLT rate and overall safety and tolerability prior to the formal identification of the RP2D.

MTD and RP2D:

The MTD is defined as the highest dose level where the observed DLT rate is acceptable (0 of at least 3 evaluable patients or a maximum of 1 in 6 evaluable with DLT). Considering all available data, the RP2D of the CART-PSMA-TGFβRDN cells to be studied in the future will be identified. The RP2D may be the same dose as the MTD or a lower dose level studied in Phase 1 and will not be a higher dose than the MTD that is identified in the Phase 1.

Definition of DLT:

In this study, adverse events (AEs) are graded according the Common Terminology Criteria for Adverse Events (CTCAE) version 5.0 with the exception of CRS. Grading of CRS is based on published American Society for Blood and Marrow Transplantation (ASBMT) criteria for CRS (Lee et al., Biol Blood Marrow Transplant (2019) 25(4): 625-638) and adapted in Table 3 below. CRS is a recognized complication of CART therapy (Porter et al., N Engl J Med (2011) 365: 725-733; Maude et al. N Engl J Med (2014) 371: 1507-1517; Teachey et al. Cancer Discov (2016) 6(6):664-679).

For the purpose of this trial, a DLT is defined in the CART-PSMA-TGFβRDN study as any CART-treatment related adverse event of Grade 3 or greater with the exception of the criteria outlined in Table 3 below.

TABLE 3
Grading Scale for CRS Events per ASBMT (Lee et al., Biol Blood
Marrow Transplant (2019) 25(4): 625-638)

| Grade | Toxicity |
|---|---|
| Grade 1 | Fever* ≥38° C. |
| Grade 2 | Fever ≥38° C. with either hypotension^{y} not requiring |
| | vasopressors and/or hypoxia^{y} requiring low-flow nasal |
| | cannula ^{z} or blow-by |
| Grade 3 | Fever ≥38° C. with either hypotension^{y} requiring 1 |
| | vasopressor with or without vasopressin and/or hypoxia^{y} |
| | requiring high-flow nasal cannula ^{z}, facemask, |
| | nonrebreather mask, or Venturi mask |
| Grade 4 | Fever ≥38° C. with either hypotension^{y} requiring multiple |
| | vasopressors (excluding vasopressin) and/or hypoxia^{y} |
| | requiring positive pressure (e.g. CPAP, BiPAP, intubation |
| | and mechanical ventilation) |
| Grade 5 | Death |

*Fever is defined as temperature ≥38° C. not attributable to any other cause. In patients who have CRS who then receive antipyretics or anticytokine therapy such as tocilizumab or steroids, fever is no longer required to grade subsequent CRS severity. In this case, CRS grading is driven by hypotension and/or hypoxia.
^{y}CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as Grade 3 CRS.
^{z} Low-flow nasal cannula is defined as oxygen delivered at 6 L/minute. Low flow also includes blow-by oxygen delivery, sometimes used in pediatrics. High-flow nasal cannula is defined as oxygen delivered at >6 L/minute.

Neurotoxicity and encephalopathy (CAR-related Encephalopathy Syndrome (CRES)) will be graded using the specific adverse event term in CTCAE version 5.0. The immune effector cell-associated encephalopathy (ICE) scoring system will be utilized in this study to monitor the following four assessments (orientation, naming, following commands and handwriting/counting):
Orientation to year, month, city, hospital (maximum of 4 points);
Name three objects for example, point to clock, pen, button (maximum of 3 points);
Follow one command, for example ‘hold up two fingers’ (1 point)
Write a standard sentence, for example, ‘our national bird is the bald eagle’ (1 point); count backwards from 100 in tens (1 point).

The ICE score is a useful tool to follow patients with neurologic toxicity to determine progression of neurotoxicity and determine management. Patients with any neurologic findings following the administration of CART-PSMA-TGFβ DN cells will be assessed using the ICE scale at a minimum of every 8-12 hours until resolution of the neurologic findings.

Management guidance is provided in Tables 4-6.

TABLE 4
DLT Criteria per ASBMT (Lee et al., Biol Blood Marrow Transplant
(2019) 25(4): 625-638)

| | |
|---|---|
| Organ System | DLT Criterion |

General
Any adverse event considered possibly or probably related to the
treatment with CART-PSMA-TGFβRDN that leads to the
discontinuation of the treatment will be considered a DLT.
Non-hematologic DLT criteria

| | |
|---|---|
| Neurologic | Any Grade 2 or higher neurotoxicity that does not |
| | resolve to Grade 1 within 72 hours considered |
| | possibly or probably related to treatment. |
| CRS | Any Grade 3 which does not resolve in 7 days to |
| | rade 0 or 1 and is considered possibly or probably |
| | related to treatment |

Hematologic toxicity
A DLT will consist of any Grade 4 adverse event possibly or probably
related to treatment with CART-PSMA-TGFβRDN that does not
resolve within 7 days of supportive treatment.

| | |
|---|---|
| Thrombocytopenia | Grade 3 or higher thrombocytopenia possibly or |
| | probably related to treatment with hemorrhage |
| | will be considered a DLT. |

CART = chimeric antigen receptor;
DLT = dose limiting toxicity

TABLE 5
Management Guidance for CRS

| CRS Grade | Toxicity | Management guidance |
|---|---|---|
| Mild | Fever, no | Supportive care indicated and frequent |
| (Grade 1) | severe | monitoring of vital signs per institutional |
| | organ | standard |
| | toxicity | NSAID for treatment of fever |
| | | Evaluation for infectious cause of fever and |
| | | initiate empiric broad-spectrum antibiotics and |
| | | filgrastim if indicated (e.g., neutropenia) |
| | | Maintenance intravenous fluid therapy |
| | | Re-assess for possible progression of grade of |
| | | CRS and need for immunosuppression (e.g., |
| | | tocilizumab) |
| Moderate | Fever and | Supportive care indicated and frequent |
| (Grade 2) | hypoxia | monitoring of vital signs per institutional |
| | | standard |
| | | NSAID for treatment of fever |
| | | Evaluation for infectious cause of fever and |
| | | initiate empiric broad-spectrum antibiotics and |
| | | filgrastim if indicated (e.g., neutropenia) |
| | | Supplemental oxygen as needed for hypoxia |
| | Hypotension | IV fluid bolus (×2 as needed for management |
| | | of hypotension) of 500-1,000 ml of normal |
| | | saline |
| | | Tocilizumab 8 mg/kg IV Q6 h for the treatment |
| | | of hypotension (maximum dose 800 mg). If |
| | | hypotension persists after fluid boluses and |
| | | tocilizumab, start vasopressors, and consider |
| | | additional monitoring needs |
| | | Consider adding corticosteroid therapy |
| | | especially with any associated neurologic |
| | | toxicity (dexamethasone 10 mg IV Q6 h). |
| Severe | Fever and | Supportive care indicated and frequent |
| (Grade 3) | hypoxia | monitoring of vital signs per institutional |
| | | standard |
| | | NSAID for treatment of fever |
| | | Evaluation for infectious cause of fever and |
| | | initiate empiric broad-spectrum antibiotics and |
| | | filgrastim if indicated (e.g., neutropenia) |
| | | Supplemental oxygen as needed for hypoxia |
| | | Supplemental oxygen including high-flow |
| | | oxygen delivery and non-invasive positive |
| | | pressure ventilation |
| | Hypotension | IV fluids, tocilizumab, vasopressors as |
| | | indicated for management of hypotension, and |
| | | hemodynamic monitoring |
| | | Consider high-dose corticosteroid therapy (e.g., |
| | | methylprednisolone 1 g/day IV, hydrocortisone |
| | | 100 mg IV) |
| | | Consider further immunosuppression with |
| | | siltuximab or anakinra as indicated |
| Life- | Fever and | Supportive care indicated and frequent |
| threatening | hypoxia | monitoring of vital signs per institutional |
| (Grade 4) | | standard |
| | | NSAID for treatment of fever |
| | | Evaluation for infectious cause of fever and |
| | | initiate empiric broad-spectrum antibiotics and |
| | | filgrastim if indicated (e.g., neutropenia) |
| | | Supplemental oxygen as needed for hypoxia |
| | | Mechanical ventilation indicated for hypoxia |
| | Hypotension | IV fluids, tocilizumab, high-dose vasopressors |
| | | as indicated for management of hypotension, |
| | | and hemodynamic monitoring |
| | | Initiate high-dose corticosteroid therapy (e.g., |
| | | methylprednisolone 1 g/day IV, hydrocortisone |
| | | 100 mg IV) |
| | | Consider further immunosuppression with |
| | | siltuximab or anakinra as indicated |

CRS = cytokine release syndrome;
IV = intravenous;
NSAID = non-steroidal anti-inflammatory drug;
Grade 5 = death.
Adapted from Neelapu et al. (2018) Nat Rev Clin Oncology, 15:47.

TABLE 6
CAR-related Encephalopathy Syndrome (CRES) Management Guidance

| Severity | |
|---|---|
| Observed | Management Guidance |
| Mild impairment | Supportive care, i.v. hydration and limiting oral |
| (ICE score 7-9) | intake |
| | Neurology evaluation and consultation (consider |
| | EEG, fundoscopic exam, brain/spine MRI) |
| | Consider early tocilizumab therapy if associated |
| | with CRS symptoms (8 mg/kg i.v. maximum dose |
| | 800 mg Q8 h as indicated) per Institutional standard. |
| | Consider early corticosteroid therapy |
| | (dexamethasone 10 mg IV Q6 h) especially in cases |
| | presenting late (weeks following CART therapy) or |
| | cases without associated CRS symptoms |
| Moderate | Supportive care, i.v. hydration and limiting oral |
| impairment | intake |
| (ICE score 3-6) | Neurology evaluation and consultation (consider |
| | EEG, fundoscopic exam, brain/spine MRI) |
| | Tocilizumab (8 mg/kg i.v. maximum dose 800 mg |
| | Q8 h as indicated) if associated with concurrent |
| | CRS. |
| | In addition to tocilizumab, add dexamethasone 10 |
| | mg i.v. Q6 h or methylprednisolone 1 mg/kg IV |
| | Q12 h per Institutional standard. |
| Severe impairment | Supportive care, IV hydration and limiting oral |
| Low ICE score, | intake |
| papilledema | Neurology evaluation and consultation (consider |
| and/or evidence of | EEG, fundoscopic exam, brain/spine MRI) |
| seizure activity | Tocilizumab (8 mg/kg i.v. maximum dose 800 mg |
| | Q8 h as indicated) if associated with concurrent |
| | CRS. In addition to tocilizumab, add dexamethasone |
| | 10 mg i.v. Q6 h or methylprednisolone 1 mg/kg i.v. |
| | Q12 h per Institutional standard. |
| | Status epilepticus management |
| | Non-convulsive: Lorazepam 0.5 mg i.v., increase |
| | dose by 0.5 mg increment to 2 mg i.v. total to |
| | control evidence of seizures. Consider maintenance |
| | dosing of levetiracetam, lorazepam and/or |
| | phenobarbital per Institutional standard. EEG |
| | monitoring |
| | Convulsive: Lorazepam 2 mg i.v. to control seizures |
| | with additional 2 mg i.v. as indicated. Consider |
| | maintenance dosing of levetiracetam, lorazepam |
| | and/or phenobarbital per Institutional standard. EEG |
| | monitoring |

ICE = Immune effector cell associated encephalopathy (modified CARTOX-10);
CRS = cytokine release syndrome;
EEG = electroencephalogram;
IV = intravenous;
MRI = magnetic resonance imaging;
Q6 h = every 6 hours;
Q12 h = every 12 hours.
Adapted from Neelapu et al. (2018) Nat Rev Clin Oncology, 15:47 and Lee et al., Biol Blood Marrow Transplant (2019) 25(4): 625-638.

Study Procedures:

Apheresis Procedures:

A large volume apheresis procedure is carried out. If the manufacturing of T cell product is not successful because it does not meet release criteria or if the target dose is not reached, patients will have the choice of a second apheresis for a second manufacturing process.

Apheresis Collection process: Apheresis will be completed according to standard clinical procedures. All patients will undergo a steady-state peripheral blood mononuclear cell leukapheresis to collect total nucleated cells (TNC). The leukapheresis collection should be approximately 10-15 liters. Collection parameters should be set to focus on lymphocytes and minimize collection of granulocytes and red blood cells. The target range of the cell collection is ≥7×10⁹ TNC, with a minimum acceptable number of 5×10⁹ TNC. From a single leukapheresis, the intention is to harvest at least 5×10⁹ white blood cells to manufacture CAR T cells. The Apheresis product is then cryopreserved.

ALC recommendation: It is recommended that the patient have an absolute lymphocyte count (ALC) ≥500/μl prior to undergoing apheresis. If the ALC is <500/μl, it is recommended that the leukapheresis be delayed until the ALC recovers. In patients with an ALC <500/μl at the time of leukapheresis, the minimum acceptable number of 5×10⁹ TNC is required as the minimum cell number to be collected.

Historical Apheresis Sample: Cryopreserved historical apheresis products collected from the patient prior to study entry can only be utilized for CART-PSMA-TGFβRDN cell product manufacturing if patients cannot undergo apheresis.

Lymphodepletion Chemotherapy:

Patients are treated with the defined lymphodepletion regimen on Days −6 to −4 (with a −1 day window) for the LD chemotherapy regimen. Prior to administration of the LD regimen, the pre-LD checklist must be assessed in each patient, set forth in Table 7.

TABLE 7
Lymphodepletion Checklist to be Completed Prior to Lymphodepletion
Regimen

| No. | Category | Description | |
|---|---|---|---|
| 1 | Disease | Patients must not have experienced new disease | |
| | response | complications, tumor response or symptoms that | |
| | | would render it unsafe to receive the LD Regimen | |
| | | and CART infusion | |
| 2 | Anti-tumor | Patients must not have received anti-tumor therapy | |
| | therapy | except lymphodepletion chemotherapy and androgen | |
| | | deprivation therapy (ADT) within 2 weeks prior to | |
| | | the administration of the CART cells. Bridging anti- | |
| | | cancer therapy must be completed at least 2 weeks | |
| | | prior to CART cell infusion | |
| 3 | ECOG | Patients must not have experienced a significant | |
| | status | change in performance or clinical status when | |
| | | compared to the status at Screening that would | |
| | | increase the risk of the CART cell infusion | |
| 4 | Respiratory | Patients must undergo the respiratory virus panel and | |
| | viral panel | results obtained within 10 days prior to the planned | |
| | | CART infusion. If the patient is positive for any virus | |
| | | on the viral panel, then anti-viral treatment should be | |
| | | commenced, and the CART cell infusion delayed at | |
| | | least 7 days following treatment. If clinical symptoms | |
| | | are present, then the LD chemotherapy and CART cell | |
| | | infusion should be delayed until resolution. If patients | |
| | | cannot receive the CART infusion within 4 weeks | |
| | | from receiving the LD regimen, then the LD | |
| | | chemotherapy should be repeated. | |
| 5 | Interval | Patients must not have any of the following toxicities | |
| | toxicity | (as a result of the bridging chemotherapy): | |
| | assessment | a. | Pulmonary: Requirement for supplemental oxygen |
| | | | to maintain the oxygen saturation of greater than |
| | | | 95% or presence of new findings on pulmonary |
| | | | imaging studies (imaging studies are not required |
| | | | prior to LD or CART infusion unless needed to |
| | | | manage toxicity) |
| | | b. | Cardiac: no new cardiac arrhythmia; congestive |
| | | | heart failure, or myocardial infarction. |
| | | c. | Hypotension requiring medical (inotropic) support |
| | | d. | Active infection defined as positive cultures for |
| | | | bacteria, virus or fungus requiring antibiotics. |
| | | | Patients who require antibiotic therapy during the |
| | | | pre-infusion, pre-LD timeframe should complete |
| | | | the antibiotic therapy prior to LD chemotherapy |
| | | | and CART infusion |

CART = chimeric antigen receptor t cells;
ECOG = Eastern Cooperative Oncology Group;
LD = lymphodepletion

CART-PSMA-TGFβRDN Infusion:

Prior to CART-PSMA-TGFβRDN administration, patients must be assessed against the Pre-CART infusion checklist (Table 8). CART-PSMA-TGFβRDN cells are infused via i.v. catheter without an in-line filter. Patients' vital signs monitored Q2 hours for the first 12 hours following infusion and Q4 hours until discharge. The total admission is approximately 3-10 days (if LD regimen administered inpatient). Additional inpatient time may be needed for treatment-related toxicities.

Following discharge, patients will remain within the local geographic region of the treating center/clinical trial site for 4 weeks following the infusion and return for specified follow-up visits. The local geographic region is defined as driving distance to the site of less than one hour. Patients will be instructed to contact the center immediately with fever greater than 38′C, nausea and vomiting, dyspnea or malaise, or any neurologic changes and may be admitted for evaluation and management of possible CRS or CRES.

TABLE 8
CART-PSMA-TGFβRDN Infusion Checklist

| No. | Category | Description | |
|---|---|---|---|
| 1 | Disease | Patients must not have experienced new disease | |
| | response | complications, tumor response or symptoms that | |
| | | would, in the opinion of the investigator, render it | |
| | | unsafe to receive the CART infusion. | |
| 2 | Anti-tumor | Patients must not have received anti-tumor therapy | |
| | therapy | except LD chemotherapy or ADT within 2 weeks prior | |
| | | to the administration of the CART cells. Bridging | |
| | | therapy must be completed at least 2 weeks prior to | |
| | | CART cell infusion. | |
| 3 | ECOG | Patients must not have experienced a significant | |
| | status | change in performance or clinical status since LD | |
| | | chemotherapy when compared to the status at | |
| | | Screening that would in the opinion of the investigator | |
| | | increase the risk of the CART cell infusion. | |
| 4 | Respiratory | Patients must undergo the RVP and results obtained | |
| | viral panel | within 10 days prior to the planned CART infusion. If | |
| | | assessed prior to LD chemotherapy, the RVP does not | |
| | | need to be re-assessed following LD chemotherapy. If | |
| | | the patient is positive for any virus on the viral panel, | |
| | | then treatment should be commenced, and the CART | |
| | | cell infusion delayed at least 7 days following | |
| | | treatment. If clinical symptoms are present, then the | |
| | | LD chemotherapy and CART cell infusion should be | |
| | | delayed until resolution. If patients cannot receive the | |
| | | CART infusion within 4 weeks from receiving the LD | |
| | | regimen, then the LD chemotherapy should be | |
| | | repeated. Consult with the Sponsor medical monitor in | |
| | | this setting. | |
| 5 | LD | Patients must not have experienced the following | |
| | regimen | toxicities during LD chemotherapy: | |
| | toxicities | e. | Pulmonary: Requirement for supplemental oxygen |
| | | | to maintain the oxygen saturation of greater than |
| | | | 95% or presence of new findings on pulmonary |
| | | | imaging studies (no imaging studies are required |
| | | | prior to lymphodepletion or CART infusion) |
| | | f. | Cardiac: no new cardiac arrhythmia, congestive |
| | | | heart failure, or myocardial infarction |
| | | g. | Hypotension requiring medical support |
| | | h. | Active infection defined as positive cultures for |
| | | | bacteria, virus or fungus requiring antibiotics. |
| | | | Patients requiring antibiotic therapy during the |
| | | | pre-infusion time period should complete therapy |
| | | | before lymphodepletion and CART infusion |

Patients experiencing toxicities from LD chemotherapy
should have these toxicities managed and resolved to
the investigator's discretion prior to the infusion of the
CART cells. If patients cannot receive the CART
infusion within 4 weeks, then LD chemotherapy
should be repeated. Consult with the Sponsor medical
monitor in this setting

CART = chimeric antigen receptor t cells;
ECOG = Eastern Cooperative Oncology Group;
LD = lymphodepletion;
RVP = respiratory viral panel

Adverse event reporting: Adverse event (AE) reporting will begin in all patients in Phase 1 at the earlier event of (1) Optional protocol-specified biopsy for inclusion during Screening; or (2) the leukapheresis procedure. Adverse event reporting for all AEs will continue through 2 years after the infusion or until patients begin an alternative cancer-related treatment, whichever comes first. While on study, patients will be continually reassessed for evidence of acute and cumulative toxicities. Specific AEs will be followed during the long-term follow up (LTFU) period as detailed below.

Periods of the Study:
A. Pre-screening: Patients without an archival sample can undergo biopsy using only non-significant risk procedures for Pre-screening sample to be collected. Patients with PSMA+ tumors by central immunohistochemistry (IHC) assay can advance to Screening.
B. Screening: Patients who meet all inclusion criteria and have none of the exclusion criteria may enter the study at the time of assignment of a dose cohort.
C. Leukapheresis: Patients meeting all trial enrollment criteria and assignment to a dosing cohort will undergo the trial and main leukapheresis procedure.
D. Bridging chemotherapy: It may be appropriate to offer a regimen of anti-cancer therapy following the leukapheresis procedure and before the LD chemotherapy regimen is administered. In general, regimens that can be administered with no long-term toxicities interfering with the administration of the LD regimen can be considered for maintenance therapy post-apheresis and pre-LD regimen. Patients must have at least 2 weeks without any therapy prior to the LD regimen administration.
E. Lymphodepletion chemotherapy: All patients will receive the LD chemotherapy regimen beginning on Day −6 through Day −4 prior to CART infusion (the window of time to administer the LD chemotherapy regimen can be extended to Day −7 with CART infusion at Day 0). Prior to LD regimen being administered, patients must be assessed against the Pre-LD Administration checklist (Table 7).
F. CART Infusion: CART infusion will begin 3-4 days after completion of LD chemotherapy (with the exception of patients in Cohort 1 who will not receive LD chemotherapy). Patients will be admitted for the CART infusion and managed as an inpatient following the CART cell infusion for at least 2 overnights observation period. Additional inpatient management may be required for toxicities observed following the CART infusion.
G. Disease Follow-up: Patients will return to the clinic on Days 3, 7, 10 (+1 day), 14, 21 and 28 and a minimum of every 8 weeks following the CART cell infusion (defined as Day 0) during the Disease follow-up period. Following the visit at 8 weeks post-CART infusion, patients will return to the treating center every 8 weeks for disease assessments or earlier to evaluate any AEs; disease assessments will continue until documented disease progression has occurred or the patient has commenced new anti-tumor treatment. Disease Imaging and PCWG3 assessments will be completed.
H. Long-term and survival follow-up: Long-term follow-up visits are determined based on the time the patient discontinues from the active Disease Follow-up period. The purpose of LTFU is to monitor all patients exposed to CART-PSMA-TGFβRDN cells to assess safety parameters such as risk of delayed CART-related AEs and monitoring for vector/CART persistence and replication competent lentivirus. Collection of long-term effects will help to better define the risk-benefit profile of PSMA and TGFbeta directed CAR T-cell therapy. Patients entering LTFU will begin these visits based on the timing of disease progression and timing of entering the LTFU period, determined from the infusion Day 0. LTFU visit frequency is set by the timing since CART infusion: from 24 months to 5 years post-CART, patients are assessed every 6 months and after 5 years patients are assessed every year to 15 years. Long-term safety follow-up visits will continue for a period of at least 15 years following administration of the CART cells. Survival follow-up will continue until the date of death is recorded. During the LTFU, patients are assessed for safety endpoints: (1) development of replication competent lentivirus (RCL); (2) persistence of the CART-PSMA-TGFβRDN in the peripheral blood; and (3) new adverse events considered potentially related to the administration of CART-PSMA-TGFβRDN. In addition to safety, patients are also assessed for OS.

Primary Objective:

Identify the recommended RP2D for further study of CART-PSMA-TGFβRDN cells that can be administered safely in patients with mCRPC with the LD chemotherapy regimen in a fixed dose regimen.

Secondary and Exploratory Objectives:

Secondary:
Assess the safety, tolerability and feasibility of CART-PSMA-TGFβRDN in mCRPC
Determine the preliminary anti-tumor efficacy of CART-PSMA-TGFβRDN in mCRPC
Correlation of the expression level of tumor PSMA expression with efficacy and safety parameters
Correlation of peripheral expansion and persistence of CART-PSMA-TGFβRDN cells with related efficacy and safety parameters

Exploratory:
Characterize the clinical pharmacology of the CART-PSMA-TGFβRDN cells with respect to their expansion, persistence, trafficking, effector status and function (e.g. cytokine release) within the periphery and the tumor microenvironment
Evaluate the pattern of tumor markers (e.g. tumor microenvironment [TME] markers of inflammation and immunosuppression) and PSA
Evaluate tumor antigen immune escape mechanisms in tumor biopsies and peripheral blood
Evaluate the development of anti-CAR immune responses
Evaluate Gallium-68 PET/CT for response assessment of PSMA+ tumor

Long Term Follow Up (15 Years Following CART Infusion):
Evaluate the incidence of new malignancies and pre-malignant conditions in all patients
Evaluate the de novo incidence of or exacerbation of pre-existing neurologic disorders in all patients
Evaluate the de novo incidence of or exacerbation of prior rheumatologic or other autoimmune disorders
Evaluate the de novo incidence of or exacerbation of prior hematologic disorders
Evaluate vector persistence by assessing the detectable CART-PSMA-TGFβRDN transgene levels in peripheral blood by q-PCR at pre-specified post CART infusion time points
Evaluate patients for the presence of detectable replication competent lentivirus (RCL) by vesicular stomatitis virus G (VSV-G)

Inclusion and Exclusion Criteria:

Enrollment of patients who have progressed on second generation androgen receptor blockers and CYP17α inhibitors, in either the hormone sensitive or hormone refractory mCRPC setting will be allowed, regardless of prior treatment with chemotherapy. All patients must have had at least 2 lines of prior therapy for metastatic prostate cancer, not including androgen deprivation therapy (GnRH agonist).

The key inclusion and exclusion criteria for this study are provided in Table 9. In addition, since PSMA-CART-TGFβDN is a targeted therapy, there will be a requirement for expression of PSMA on an archival biopsy, or fresh tissue tumor biopsy, taken only if the procedure is considered to be a nonsignificant risk for the patient.

TABLE 9
Inclusion and Exclusion Criteria

| Population | Criteria | | |
|---|---|---|---|
| Key | The inclusion criteria are described below. | | |
| Inclusion | 1. | Patients must be adults over 18 years of age and sign | |
| Criteria | | the main study informed consent form | |
| | 2. | Patients must have a confirmed diagnosis of metastatic | |
| | | CRPC | |
| | 3. | Patients must have a testosterone level <50 ng/ml | |
| | 4. | Patients must have received at least 2 prior lines of | |
| | | therapy for metastatic prostate cancer, including at least | |
| | | one second generation androgen receptor inhibitor (e.g. | |
| | | enzalutamide or apalutamide) and/or CYP17α inhibitor | |
| | | (e.g. abiraterone/prednisone). Androgen deprivation | |
| | | therapy (ADT) with GnRH does not count as a line of | |
| | | therapy. | |
| | 5. | Patients must have evidence of progressive castrate | |
| | | resistant prostate adenocarcinoma, as defined by: | |
| | | a. Castrate levels of testosterone (<50 ng/ml) AND | |
| | | b. Evidence of one of the following measures of | |
| | | progressive disease in the 12 weeks preceding eligibility | |
| | | confirmation by physician: | |
| | | | i. soft tissue progression by RECIST 1.1 criteria |
| | | | ii. osseous disease progression with 2 or more new |
| | | | lesions on bone scan |
| | | | iii. increase in serum PSA of at least 25% and an |
| | | | absolute increase of 2 ng/ml or more from nadir on |
| | | | at least three consecutive tests a minimum of 1 week |
| | | | apart |
| | 6. | Adequate vital organ function as defined by | |
| | | a. | Estimated creatinine clearance 30 ml/min by MDRD |
| | | b. | ALT and AST ≤3× the upper limit of normal and |
| | | | total bilirubin ≤2.0 mg/dL. |
| | | c. | Serum total bilirubin <1.5 × ULN unless patient has |
| | | | known Gilbert's Syndrome, and no other reason for |
| | | | indirect bilirubinemia |
| | | d. | Serum albumin ≥_3.0 g/dL |
| | | e. | Left ventricular ejection fraction (LVEF) ≥45%. |
| | | | LVEF assessment must have been performed within |
| | | | 8 weeks of enrollment |
| | 7. | Adequate hematologic reserve (without the use of | |
| | | supportive transfusion or hematopoietic growth factors | |
| | | within 4 weeks of apheresis), as defined by | |
| | | a. | Hemoglobin ≥9 g/dL |
| | | b. | Absolute neutrophil count ≥1000/μL |
| | | c. | Platelet count ≥100,000/μL |
| | | | Note: patients must not be transfusion-dependent to |
| | | | maintain hematologic parameters |

| | 8. | All patients must have PSMA+ disease, determined by |
|---|---|---|
| | | centrally tested PSMA IHC expression in an archival |
| | | tumor biopsy. If an archival tumor biopsy sample is not |
| | | available, then the patient may undergo a biopsy for |
| | | the purposes of screening eligibility with only non- |
| | | significant risk biopsy procedures. |
| | 9. | Patients who have not undergone bilateral orchiectomy |
| | | must be able to continue GnRH therapy during the |
| | | study. |
| | 10. | All patients must have evaluable disease per Prostate |
| | | Cancer Working Group (PCWG3) criteria. |
| | 11. | Patients must have an Easter Cooperative Oncology |
| | | Group (ECOG) score of 0 or 1 |
| | 12. | Toxicities from any previous therapy must have |
| | | recovered to Grade 1 or baseline. |
| | 13. | Life expectancy greater than 3 months |
| | 14. | Patients of reproductive potential agree to use approved |
| | | contraceptive methods per protocol. |
| Key | The exclusion criteria are described below. | |
| Exclusion | 1. | Active invasive cancer, other than the proposed cancer |
| Criteria | | included in the study, within 2 years prior to screening, |
| | | unless treated with curative intent, i.e. nonmelanoma |
| | | skin cancer. |
| | 2. | Current treatment with systemic steroids (defined as a |
| | | dose greater than the equivalent of prednisone 10 |
| | | mg/day). Low-dose physiologic replacement therapy |
| | | with corticosteroids equivalent to prednisone 10 mg/day |
| | | or lower, topical steroids, and inhaled steroids are |
| | | acceptable. |
| | 3. | Active autoimmune disease (including connective tissue |
| | | disease, uveitis, sarcoidosis, inflammatory bowel |
| | | disease or multiple sclerosis) or a history of severe |
| | | autoimmune disease requiring prolonged |
| | | immunosuppressive therapy. Patients should have |
| | | stopped any immunosuppressive therapy within 6 |
| | | weeks prior to screening visit. |
| | 4. | Current, active HIV, HCV, HBV infections. Viral |
| | | testing at Screening is required in all patients to rule out |
| | | subclinical infections. Patients who are HBcAb positive |
| | | are excluded, even if HBSAg negative. |
| | 5. | Other active or uncontrolled medical or psychiatric |
| | | condition that would preclude participation. |
| | 6. | Prior allogeneic stem cell transplant. |
| | 7. | Active and untreated central nervous system (CNS) |
| | | malignancy. Treated lesions may be considered inactive |
| | | if they are stable for at least 1 month following |
| | | definitive treatment. Patient must not require |
| | | corticosteroid therapy or anti-epileptic medications for |
| | | the management of brain metastases. |
| | 8. | History of severe infusion reaction to monoclonal |
| | | antibodies or biological therapies, or to study product |
| | | excipients (e.g. human serum albumin, dimethyl |
| | | sulfoxide (DMSO), dextran 40) that would preclude the |
| | | patient safely receiving CART-PSMA-TGFβRDN cells. |
| | 9. | History of being previously treated with a J591 |
| | | antibody-based therapy |
| | 10. | Active or recent (within the past 6 months prior to |
| | | apheresis) cardiac disease, defined as (1) New York |
| | | Heart Association (NYHA) Class III or IV heart failure, |
| | | (2) unstable angina or (3) a history of recent (within 6 |
| | | months) myocardial infarction or sustained (>30 |
| | | second) ventricular tachyarrhythmias. |
| | 11. | Have inadequate venous access for or contraindications |
| | | for the apheresis procedure. |

Clinical Trial Assay PSMA Expression Analysis:

Patients will be screened via an investigational immunohistochemistry (IHC) assay for enrollment to the CART-PSMA-TGFβRDN clinical trial. Archival tissue, or fresh biopsy, taken only if the procedure is considered to be a nonsignificant risk for the patient, will be used for the IHC test of PSMA-expression. An analytically validated clinical trial assay (CTA) will be used for the pre-screening of patients for PSMA-positive status at a central CAP-CLIA certified laboratory. This IHC assay utilizes a PSMA specific antibody for the assessment of PSMA expression in formalin-fixed paraffin-embedded tumor samples.

Manufacturing:

The CART-PSMA-TGFβRDN investigational product is a genetically modified autologous T-cell immunotherapy. The autologous T-cells are transduced by lentivirus vector to express a dominant negative variation of the type II transforming growth factor beta receptor (TGFβRDN) and a second-generation chimeric antigen receptor (CAR) specific to the prostate specific membrane antigen (PSMA) protein. The TGFβRIIDN transgene has been generated by truncation of a cytoplasmic portion of type II transforming growth factor beta receptor (TGFβRII) and the CAR construct has been generated by fusion of a PSMA-specific single-chain variable region fragment (scFv) with human CD3ζ and 4-1BB signaling domains. The anti-PSMA scFv was derived from publicly available sequences of the murine J591 anti-PSMA antibody. The construct is bicistronic, which allows nearly equivalent expression of both transgenes from the same vector. The CAR construct used for CART-PSMA-TGFβRDN is shown in FIG. 1A and the structure of the CAR of CART-PSMA-TGFβRDN is shown in FIG. 1B.

CART-PSMA-TGFβRDN is prepared from patient peripheral blood mononuclear cells, which are obtained via a standard leukapheresis procedure. Based on the constitution of the leukapheresis product, the following processes may occur: depletion of monocytes via counterflow centrifugal elutriation, washing step, and/or Ficoll separation of the PBMCs. T-cells are enriched from the autologous leukapheresis, activated with anti-CD3 and anti-CD28 antibody coated magnetic beads followed by transduction with the lentiviral vector containing the PSMA-TGFbRIIDN CAR transgene. The transduced T-cells are expanded in cell culture, beads removed, washed, and formulated into a suspension according to the assigned cohort based on the clinical protocol, and then cryopreserved. The manufacturing process for CART-PSMA-TGFβRDN is a continuous process with no holding step from drug substance to drug product.

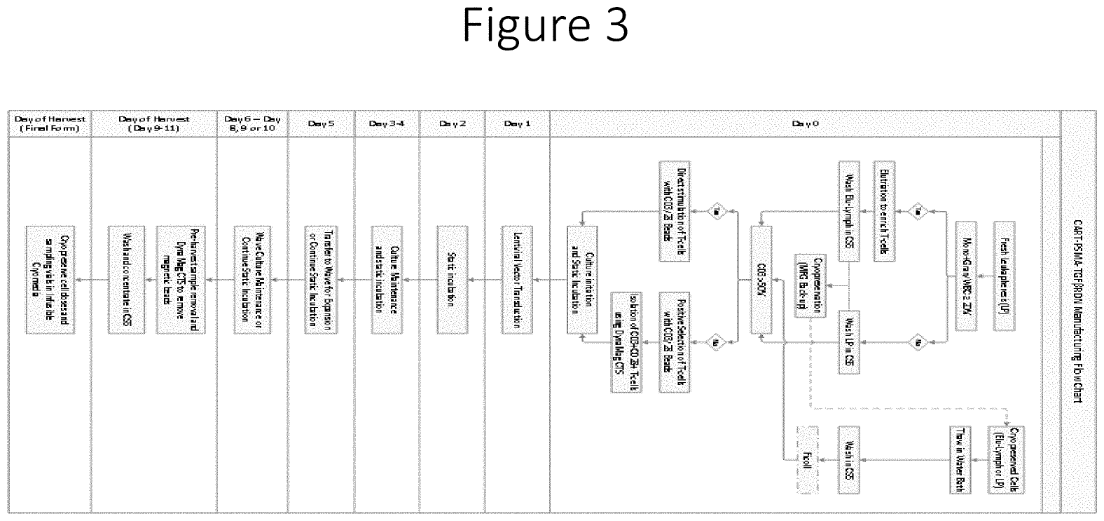

FIG. 3 shows an overview of the CART-PSMA-TGFβRDN manufacturing process. The key equipment per unit operation is listed in Table 10. The critical raw materials and excipients are listed in Table 11. The investigational product is stored frozen and thawed prior to administration.

TABLE 10
Key Equipment Used in the CART-PSMA-TGFβRDN
Manufacturing Process

| Key Equipment | Unit Operation(s) |
|---|---|
| Water bath | Apheresis thaw |
| TerumoBCT Elutra ® | Elutriation |
| Cell Saver ® 5 Plus | Apheresis product wash |
| | Harvest wash and concentrate |
| CTS ™ DynaMag ™ | T-cell enrichment |
| Magnet | Bead removal |
| CO2 Incubator | Culture expansion |
| GE Cellbag | Culture expansion |
| Controlled-rate freezer | Cryopreservation |

TABLE 11
Critical Raw Materials and Excipients

| Critical Raw Materials/Excipients | Use |
|---|---|
| Plasma-Lyte A | Elutriation |
| | Harvest |
| | Infusible cryomedia |
| 5% Human Serum Albumin | Elutriation |
| 5% Dextrose in 0.45% sodium chloride | Elutriation |
| | Harvest |
| | Infusible cryomedia |
| N-Acetylcysteine | Elutriation |
| | Culture expansion |
| XVIVO ™ 15 serum-free hematopoietic | Culture expansion |
| cell medium | |
| HEPES | Culture expansion |
| L-GlutaMAX ™ | Culture expansion |
| Human AB serum, heat inactivated | Culture expansion |
| Sodium pyruvate | Culture expansion |
| MEM vitamin solution | Culture expansion |
| Pluronic F-168 | Culture expansion |
| IL-2 (recombinant) | Culture expansion |
| CTS ™ Dynabeads ™ CD3/CD28 | Cell stimulation & |
| | activation |
| 0.9% Sodium chloride | Harvest |
| 10% Dextran 40 in 5% Dextrose | Infusible cryomedia |
| 25% Human serum albumin | Infusible cryomedia |
| Dimethylsulfoxide (DMSO) | Infusible cryomedia |

Table 12 lists the proposed specifications for the CART-PSMA-TGFβRDN investigational product.

TABLE 12
Specifications for CART-PSMA-TGFβRDN Investigational Product

| Test | Method | Acceptance Criteria |
|---|---|---|
| Cell viability on | Membrane integrity | ≥70% |
| sentinel vial | | |
| % CD3 + CD45 + | Flow cytometry | ≥80% |
| Positive T-cells | | |
| Residual bead number | Visual | ≤100 beads/3 × |
| | | 10⁶ cells |
| Endotoxin | Endosafe ® | 3.5 EU/mL |
| Mycoplasma | MycoAlert ™ PLUS | Negative |
| | Assay | |
| Transduction | Flow cytometry | ≥2% |
| efficiency | | |
| (% scFv + CD3 + | | |
| CD45+) | | |
| Sterility | BACTEC ™ culture assay | No growth at 7 |
| | | days |
| Fungal culture | Brain heart infusion agar | No growth at 7 |
| | fungal assay | days |
| Vector DNA Sequence | Q-PCR^{a} | 0.02-5 average |
| (Vector Copy Number) | | copies/cell |
| RCL (VSV-G DNA) | Q-PCR | <50 average copies |
| | | VSV-G/μg DNA |

^{a}Q-PCR: quantitative polymerase chain reaction

The CART-PSMA-TGFβRDN investigational product is resuspended in infusible cryopreservation media which contains the following:
31.25% (v/v) of Plasma-Lyte A
31.25% (v/v) of 5% Dextrose in 0.45% Sodium Chloride
10% (v/v) of 10% Dextran 40 in 5% Dextrose
20% (v/v) of 25% Human Serum Albumin (HSA)
7.5% (v/v) Dimethylsulfoxide (DMSO)

The drug product is supplied as a frozen suspension of genetically modified autologous T-cells in ethylene vinyl acetate (EVA) infusion bag(s) labeled for the specific recipient. Dosing is formulated based on anti-PSMA CAR expression on the T cells, i.e. the number of transduced cells (tr cells). The total dose of tr cells will be formulated according to the cohort assignment in the protocol. The drug product is frozen in cryopreservation bag(s) using a controlled-rate freezer at an ideal final concentration of 1×10⁷ to 1×10⁸ total nucleated cells per mL, or with a minimum of 10 mL cell suspension per bag. The drug product should be stored frozen in a cryogenic freezer at less than or equal to minus 130° C. in a temperature-monitored system until the day of patient infusion. In addition to T-cells, other cell populations, including monocytes, NK cells, and B cells, may be present.

### Example 2: A Study of CART-PSMA-TGFβRDN in Patients with Metastatic Castrate Resistant Prostate Cancer

Brief Summary:

A multi-center, open-label, Phase 1 study of the safety, tolerability and feasibility of dosing patients harboring metastatic castrate resistant prostate cancer (mCRPC) with genetically modified autologous T cells (CART-PSMA-TGFβRDN cells) engineered to express a chimeric antigen receptor (CAR) capable of recognizing the tumor antigen prostate-specific membrane antigen (PSMA) and activating the T cell.

Detailed Description:

This is a Phase 1 single-arm study designed to identify the dose and regimen of CART-PSMA-TGFβRDN cells that can be safely administered intravenously following the lymphodepletion (LD) regimen to patients with mCRPC. It is anticipated that approximately 18 patients will enroll in this study.

Table 13 lists the arms and interventions of the study.

TABLE 13
Arms and Interventions

| Arm | Intervention/treatment |
|---|---|
| Experimental: Dose Escalation | Biological/Vaccine: CART-PSMA- |
| Dose escalation of intravenous | TGFβRDN |
| CART-PSMATGFβRDN cells | Intravenous administration of |
| for patients with metastatic | genetically modified autologous T cells |
| castration resistant prostate | engineered to express a CAR capable of |
| cancer | recognizing the tumor antigen prostate- |
| | specific membrane antigen (PSMA) and |
| | a dominant negative TGFβ receptor |
| | Drug: Cyclophosphamide |
| | Patients will receive cyclophosphamide |
| | and fludarabine lymphodepletion |
| | chemotherapy followed by the |
| | investigational product, CART-PSMA- |
| | TGFβRDN |
| | Drug: Fludarabine |
| | Patients will receive cyclophosphamide |
| | and fludarabine lymphodepletion |
| | chemotherapy followed by the |
| | investigational product, CART- |
| | PSMATGFβRDN |

Outcome Measures:

Primary Outcome Measure:
1. Dose identification of CART-PSMA-TGFβRDN
   Incidence of dose limiting toxicity (DLT).

Secondary Outcome Measure:
1. Safety of CART-PSMA-TGFβRDN
   Percentage of patients experience adverse events (AEs), including serious and severe AEs overall, by dose level, and severity grade.
2. Tolerability of CART-PSMA-TGFβRDN
   Frequency of treatment emergent AEs, frequency of clinical changes from baseline in vital signs, changes in electrocardiogram, and hematology and chemistry laboratory shifts from baseline.
   3. Preliminary efficacy of CART-PSMA-TGFβRDN as assessed by biochemical Objective Response Rate (ORR)
   ORR defined as the proportion of patients with maximal prostate-specific antigen (PSA) decline of greater than or equal to 5000 at 12 weeks post infusion.
4. Feasibility of CART-PSMA-TGFβRDN
   Proportion of patients who did not receive CART-PSMA-TGFβRDN cells.
5. Peripheral expansion and persistence of CART-PSMA-TGFβRDN
   Correlation with related efficacy and safety parameters.

Table 14 outlines the patient eligibility criteria of the study.

TABLE 14
Eligibility Criteria
Ages eligible for study: 18 years or older
Sexes eligible for study: Male
Gender based: No
Accepts healthy volunteers: No

| | |
|---|---|
| Inclusion | Confirmed histologic diagnosis of prostate cancer and have |
| Criteria | mCRPC |
| | Prior therapies defined as at least 2 prior lines of systemic |
| | therapy for prostate cancer, including at least one second |
| | generation androgen receptor inhibitor and/or CYP17α |
| | inhibitor. At least one line of prior therapy must be in the |
| | mCRPC setting |
| | Evidence of disease as defined as castrate levels of |
| | testosterone (<50 ng/mL) AND Evidence of one of the |
| | following measures of progressive disease in the 12 weeks |
| | preceding eligibility confirmation by physician: i.) Soft |
| | tissue progression by Response Evaluation Criteria in Solid |
| | Tumors (RECIST) 1.1 criteria; ii.) Osseous disease |
| | progression with 2 or more new lesions on bone scan; iii.) |
| | Increase in serum PSA of at least 25% and an absolute |
| | increase of 2 ng/mL or more from nadir on at least three |
| | consecutive tests a minimum of 1 week apart |
| | PSMA+ disease determined by centrally tested PSMA |
| | expression in prior or archival tumor sample |
| | Evaluable disease per Prostate Working Group 3 (PCWG3) |
| | criteria |
| | Eastern Cooperative Oncology Group (ECOG) score of 0 |
| | or 1 |
| | Life expectancy of greater than 3 months |
| | Toxicities from any previous therapy must have recovered to |
| | Grade 1 or baseline |
| | Patients who have not undergone bilateral orchiectomy must |
| | be able to continue gonadotropin-releasing hormone (GnRH) |
| | therapy during the study |
| | Estimated creatinine clearance ≥60 mL/min by Modification |
| | of Diet in Renal Disease criteria |
| | Alanine aminotransferase (ALT) and aspartate |
| | aminotransferase (AST) ≤2.5× the upper limit of normal |
| | (ULN); patients with hepatic metastases ALT and AST |
| | ≤3.0× ULN |
| | Serum total bilirubin <1.5 mg/dL unless patient has known |
| | Gilbert's Syndrome, then serum bilirubin ≤3 mg/dL |
| | Serum albumin ≥3.0 g/dL |
| | Left ventricular ejection fraction (LVEF) ≥50%. LVEF |
| | assessment must have been performed within 8 weeks of |
| | enrollment |
| | Hemoglobin ≥9 g/dL |
| | Absolute neutrophil count ≥1500/μL |
| | Platelet count ≥100,000/μL |
| | Patients of reproductive potential agree to use of approved |
| | highly effective contraceptive methods |
| Exclusion | Active invasive cancer, other than the proposed cancer |
| Criteria | included in the study, within 2 years prior to screening, |
| | unless treated with curative intent |
| | Current treatment with systemic corticosteroids (defined as a |
| | dose greater than the equivalent of prednisone 10 mg/day) |
| | Active autoimmune disease (including connective tissue |
| | disease, uveitis, sarcoidosis, inflammatory bowel disease or |
| | multiple sclerosis) or a history of severe autoimmune |
| | disease requiring prolonged immunosuppressive therapy |
| | (any immunosuppressive therapy within 6 weeks prior to |
| | screening visit) |
| | Current, active human immunodeficiency virus (HIV), |
| | hepatits C virus, hepatitis B virus infections |
| | Prior allogeneic stem cell transplant |
| | Active and untreated central nervous system malignancy |
| | History of severe infusion reaction to monoclonal |
| | antibodies or biological therapies, or to study product |
| | excipients that would preclude the patient safely receiving |
| | CART-PSMA-TGFβRDN cells |
| | History of being previously treated with a J591 antibody- |
| | based therapy |
| | Active or recent (within the past 6 months prior to |
| | apheresis) cardiac disease, defined as (1) New York Heart |
| | Association Class III or IV heart failure, (2) unstable |
| | angina or (3) a history of recent (within 6 months) |
| | myocardial infarction or sustained (>30 second) ventricular |
| | tachyarrhythmias |
| | Have inadequate venous access for or contraindications for |
| | the apheresis procedure |
| | Must agree not to participate in a conception process or |
| | must agree to a highly effective method of contraception |

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A method of treating a solid tumor in a subject in need thereof, comprising:
(a) administering to a subject a first dose of cells, wherein the cells comprise a chimeric antigen receptor (CAR) having affinity for a solid tumor antigen, and wherein the first dose comprises about 30% of a total dose of cells; and
(b) administering to the subject a consecutive dose of cells comprising the CAR, wherein the consecutive dose comprises about 70% of the total dose of cells, and wherein the consecutive dose is administered at least five days after the administration of the first dose.

2. The method of claim 1, wherein:
(a) the first dose comprises from about 3×10⁷ cells to about 6×10⁷ cells, and the second dose comprises from about 7×10⁷ cells to about 1.4×10⁸ cells; and/or
(b) the first dose comprises from about 1.5×10⁸ cells to about 1.8×10⁸ cells, and the second dose comprises from about 3.5×10⁸ cells to about 4.2×10⁸ cells.

3. The method of claim 1, further comprising administering to the subject a lymphodepleting chemotherapy.

4. The method of claim 3, wherein the lymphodepleting chemotherapy comprises:
(a) a therapeutically effective amount of cyclophosphamide and/or fludarabine; and/or
(b) a therapeutically effective amount of cyclophosphamide and fludarabine; and/or
(c) a therapeutically effective amount of cyclophosphamide and/or fludarabine, wherein the therapeutically effective amount of cyclophosphamide is 300 mg/m²/day; and/or
(d) a therapeutically effective amount of cyclophosphamide and/or fludarabine, wherein the therapeutically effective amount of fludarabine is 30 mg/m²/day.

5. The method of claim 3, wherein the lymphodepleting chemotherapy:
(a) is administered to the subject prior to administering the first dose of cells;
(b) is administered to the subject four to six days prior to administering the first dose of cells; and/or
(c) is administered to the subject consecutively for three days.

6. The method of claim 1, wherein:
(a) the solid tumor is a prostate cancer; and/or
(b) the solid tumor is a prostate cancer and the prostate cancer is metastatic castrate resistant prostate cancer; and/or
(c) the solid tumor is a lung cancer; and/or
(d) the solid tumor is a lung cancer and wherein the lung cancer is non-small cell lung cancer; and/or
(e) the solid tumor is a breast cancer; and/or
(f) the solid tumor is a breast cancer, wherein the breast cancer is triple negative breast cancer; and/or
(g) the solid tumor is a pancreatic cancer; and/or
(h) the solid tumor is a pancreatic cancer, wherein the pancreatic cancer is pancreatic adenocarcinoma; and/or
(i) the solid tumor is an ovarian and fallopian tube cancer.

7. The method of claim 1, wherein the solid tumor antigen is:
(a) mucin-1 (MUC1); or
(b) a truncated glycoepitope of MUC1; or
(c) prostate-specific membrane antigen (PSMA).

8. The method of claim 1, wherein:
(a) the cells comprising the CAR further comprise a dominant negative receptor;
and/or
(b) the cells comprising the CAR further comprise a dominant negative receptor and the dominant negative receptor is a truncated variant of a wild-type protein associated with an immunosuppressive signal; and/or
(c) the cells comprising the CAR further comprise a dominant negative receptor and the dominant negative receptor is a truncated variant of a TGFβ receptor; and/or
(d) the cells comprising the CAR further comprise a dominant negative receptor and the dominant negative receptor is a truncated variant of a TGFβ receptor, wherein the TGFβ receptor is TGFβ receptor type II.

9. The method of claim 1 further comprising monitoring the development of cytokine release syndrome, immune cell-associated neurologic toxicities, and/or an on-target off-tumor toxicity resulting from the administration of the first dose.

10. The method of claim 9, wherein:
(a) the on-target off-tumor toxicity is parotiditis, and/or a neurologic toxicity associated with the expression of PSMA in a normal tissue; and/or
(b) the on-target off-tumor toxicity is parotiditis and/or the neurologic toxicity associated with the expression of PSMA in a normal tissue is monitored by a physical examination of the subject, optionally wherein the physical examination comprises assessing the subject for pain or glandular dysfunction; and/or
(c) the on-target off-tumor toxicity is parotiditis and the consecutive dose is administered at a time when the parotiditis and/or the neurologic toxicity associated with the expression of PSMA in a normal tissue has been treated; and/or
(d) the on-target off-tumor toxicity is parotiditis and the consecutive dose is administered at a time when the parotiditis and/or the neurologic toxicity associated with the expression of PSMA in a normal tissue has subsided; and/or
(e) the on-target off-tumor toxicity is parotiditis, and/or a neurologic toxicity associated with the expression of PSMA in a normal tissue, and wherein the normal tissue is a salivary gland and/or the hypothalamus:
(f) the on-target off-tumor toxicity is pancreatitis, renal insufficiency, and/or gastrointestinal inflammation; and/or
(g) the on-target off-tumor toxicity is pancreatitis, renal insufficiency, and/or gastrointestinal inflammation, wherein the pancreatitis, renal insufficiency, and/or gastrointestinal inflammation is monitored by a physical examination or by assessing the blood levels of amylase and/or lipase in the subject after receiving the first dose of cells, compared to the blood levels of amylase and/or lipase of the subject prior to receiving the first dose of cells, optionally wherein the physical examination comprises assessing the subject for abdominal pain; and/or
(h) the on-target off-tumor toxicity is pancreatitis, renal insufficiency, and/or gastrointestinal inflammation, wherein the consecutive dose is administered at a time when the pancreatitis, renal insufficiency, and/or gastrointestinal inflammation has been treated; and/or
(i) the on-target off-tumor toxicity is pancreatitis, renal insufficiency, and/or gastrointestinal inflammation, wherein the consecutive dose is administered at a time when the pancreatitis, renal insufficiency, and/or gastrointestinal inflammation has subsided.

11. A method of treating metastatic castrate resistant prostate cancer in a subject in need thereof, comprising:
(a) administering to a subject a first dose of cells, wherein the cells comprise a chimeric antigen receptor (CAR) having affinity for prostate-specific membrane antigen (PSMA-CAR), and a truncated variant of TGFβ receptor type II (dnTGFβR2), and wherein the first dose comprises about 30% of a total dose of cells; and administering to the subject a consecutive dose of cells comprising the PSMA-CAR and dnTGFβR2, wherein the consecutive dose comprises about 70% of the total dose of cells, and wherein the consecutive dose is administered at least five days after the administration of the first dose; and/or
(b) administering to a subject a first dose of T cells, wherein the T cells comprise a chimeric antigen receptor (CAR) having affinity for prostate-specific membrane antigen (PSMA-CAR), and a truncated variant of TGFβ receptor type II (dnTGFβR2), and wherein the first dose comprises from about 3×10⁷ cells to about 6×10⁷ cells; and administering to the subject a consecutive dose of T cells comprising the PSMA-CAR and dnTGFβR2, wherein the consecutive dose comprises from about 7×10⁷ cells to about 1.4×10⁸ cells, and wherein the consecutive dose is administered at least five days after the administration of the first dose;
and/or
(c) administering to a subject a first dose of cells, wherein the cells comprise a chimeric antigen receptor (CAR) having affinity for prostate-specific membrane antigen (PSMA-CAR), and a truncated variant of TGFβ receptor type II (dnTGFβR2), and wherein the first dose comprises from about 1.5×10⁸ cells to about 1.8×10⁸ cells; and administering to the subject a consecutive dose of cells comprising the PSMA-CAR and dnTGFβR2, wherein the consecutive dose comprises from about 3.5×10⁸ cells to about 4.2×10⁸, and wherein the consecutive dose is administered at least five days after the administration of the first dose.

12. The method of claim 11, further comprising monitoring the development of cytokine release syndrome, immune cell-associated neurologic toxicities, and/or an on-target off-tumor toxicity resulting from the administration of the first dose.

13. The method of claim 12, wherein:
(a) the on-target off-tumor toxicity is parotiditis, and/or a neurologic toxicity associated with the expression of PSMA in a normal tissue; and/or
(b) the on-target off-tumor toxicity is parotiditis, and/or a neurologic toxicity associated with the expression of PSMA in a normal tissue, wherein the parotiditis and/or a neurologic toxicity associated with the expression of PSMA in a normal tissue is monitored by a physical examination of the subject, optionally wherein the physical examination comprises assessing the subject for pain or glandular dysfunction; and/or
(c) the on-target off-tumor toxicity is parotiditis, and/or a neurologic toxicity associated with the expression of PSMA in a normal tissue, wherein the normal tissue is a salivary gland and/or hypothalamus.

14. A method of treating a solid tumor in a subject in need thereof, comprising:
(a) administering to a subject a first dose of cells, wherein the cells comprise a chimeric antigen receptor (CAR) having affinity for a solid tumor antigen, and wherein the first dose comprises about 30% of a total dose of cells;
(b) monitoring the development of cytokine release syndrome, immune cell-associated neurologic toxicities, and/or an on-target off-tumor toxicity resulting from the administration of the first dose; and
(c) administering to the subject a consecutive dose of cells comprising the CAR, wherein the consecutive dose comprises about 70% of the total dose of cells, and wherein the consecutive dose is administered at least five days after the administration of the first dose.

15. The method of claim 14, wherein:
(a) the solid tumor is a prostate cancer; and/or
(b) the solid tumor is a prostate cancer, and the prostate cancer is metastatic castrate-resistant prostate cancer; and/or
(c) the solid tumor is a prostate cancer, and the solid tumor antigen is prostate-specific membrane antigen (PSMA); and/or
(d) the solid tumor is a pancreatic cancer; and/or
(e) the solid tumor is a pancreatic cancer, wherein the pancreatic cancer is pancreatic adenocarcinoma; and/or
(c) the solid tumor is a pancreatic cancer and the solid tumor antigen is mucin-1 (MUC1); and/or
(d) the solid tumor is a pancreatic cancer and the solid tumor antigen is a truncated glycoepitope of MUC1.

16. The method of claim 15, wherein:
(e) the solid tumor is a pancreatic cancer and the on-target off-tumor toxicity is pancreatitis, renal insufficiency, and/or gastrointestinal inflammation; and/or
(b) the solid tumor is a pancreatic cancer and the on-target off-tumor toxicity is pancreatitis, renal insufficiency, and/or gastrointestinal inflammation, wherein the pancreatitis, renal insufficiency, and/or gastrointestinal inflammation is monitored by a physical examination or by assessing the blood levels of amylase and/or lipase in the subject after receiving the first dose of cells, compared to the blood levels of amylase and/or lipase of the subject prior to receiving the first dose of cells, optionally wherein the physical examination comprises assessing the subject for abdominal pain.

17. The method of claim 14, wherein:
(a) the cells comprising the CAR further comprise a dominant negative receptor; and/or
(b) the cells comprising the CAR further comprise a dominant negative receptor, wherein the dominant negative receptor is a truncated variant of a wild-type protein associated with an immunosuppressive signal; and/or
(c) the cells comprising the CAR further comprise a dominant negative receptor, wherein the dominant negative receptor is a truncated variant of a TGFβ receptor; and/or
(d) the cells comprising the CAR further comprise a dominant negative receptor, wherein the dominant negative receptor is a truncated variant of a TGFβ receptor which is TGFβ receptor type II.

18. The method of claim 14, wherein:
(a) the on-target off-tumor toxicity is parotiditis, and/or a neurologic toxicity associated with the expression of PSMA in a normal tissue; and/or
(b) the on-target off-tumor toxicity is parotiditis, and/or a neurologic toxicity associated with the expression of PSMA in a normal tissue, wherein the parotiditis and/or a neurologic toxicity associated with the expression of PSMA in a normal tissue is monitored by a physical examination of the subject, optionally wherein the physical examination comprises assessing the subject for pain or glandular dysfunction; and/or
(c) the on-target off-tumor toxicity is parotiditis, and/or a neurologic toxicity associated with the expression of PSMA in a normal tissue, wherein the normal tissue is a salivary gland and/or hypothalamus.

19. The method of claim 14, wherein:
(a) the consecutive dose is administered at a time when the on-target off-tumor toxicity has been treated; and/or
(b) wherein the consecutive dose is administered at a time when the on-target off-tumor toxicity has subsided.

20. A method of treating metastatic castrate resistant prostate cancer in a subject in need thereof, comprising:
(a) (i) administering to a subject a first dose of cells, wherein the cells comprise a chimeric antigen receptor (CAR) having affinity for prostate-specific membrane antigen (PSMA-CAR), and a truncated variant of TGFβ receptor type II (dnTGFβR2), and wherein the first dose comprises about 30% of a total dose of cells; (ii) monitoring the development of parotiditis, and/or a neurologic toxicity associated with the expression of PSMA in a normal tissue resulting from the administration of the first dose; and (iii) administering to the subject a consecutive dose of cells comprising the PSMA-CAR and dnTGFβR2, wherein the consecutive dose comprises about 70% of the total dose of cells, and wherein the consecutive dose is administered at least five days after the administration of the first dose; and/or
(b) (i) administering to a subject a first dose of T cells, wherein the T cells comprise a chimeric antigen receptor (CAR) having affinity for prostate-specific membrane antigen (PSMA-CAR), and a truncated variant of TGFβ receptor type II (dnTGFβR2), and wherein the first dose comprises from about 3×10⁷ cells to about 6×10⁷ cells; (ii) monitoring the development of parotiditis, and/or a neurologic toxicity associated with the expression of PSMA in a normal tissue resulting from the administration of the first dose; and (iii) administering to the subject a consecutive dose of T cells comprising the PSMA-CAR and dnTGFβR2, wherein the consecutive dose comprises from about 7×10⁷ cells to about 1.4×10⁸ cells, and wherein the consecutive dose is administered at least five days after the administration of the first dose; and/or
(c) (i) administering to a subject a first dose of cells, wherein the cells comprise a chimeric antigen receptor (CAR) having affinity for prostate-specific membrane antigen (PSMA-CAR), and a truncated variant of TGFβ receptor type II (dnTGFβR2), and wherein the first dose comprises from about 1.5×10⁸ cells to about 1.8×10⁸ cells; (ii) monitoring the development of parotiditis, and/or a neurologic toxicity associated with the expression of PSMA in a normal tissue resulting from the administration of the first dose; and (iii) administering to the subject a consecutive dose of cells comprising the PSMA-CAR and dnTGFβR2, wherein the consecutive dose comprises from about 3.5×10⁸ cells to about 4.2×10⁸, and wherein the consecutive dose is administered at least five days after the administration of the first dose.

21. The method of claim 20, wherein the consecutive dose:
(a) is administered at a time when the parotiditis and/or a neurologic toxicity associated with the expression of PSMA in a normal tissue has been treated; and/or
(b) is administered at a time when the parotiditis and/or a neurologic toxicity associated with the expression of PSMA in a normal tissue has subsided.

22. A method of treating a cancer in a subject in need thereof, comprising:
(a) administering to a subject a first dose of cells, wherein the cells comprise a chimeric antigen receptor (CAR) having affinity for a truncated glycoepitope of mucin-1 (TnMUC1), and wherein the first dose comprises 30% of a total dose of cells;
(b) monitoring the development of pancreatitis, renal insufficiency, and/or gastrointestinal inflammation resulting from the administration of the first dose; and
(c) administering to the subject a consecutive dose of cells comprising the TnMUC1-CAR, wherein the consecutive dose comprises 70% of the total dose of cells, and wherein the consecutive dose is administered at least five days after the administration of the first dose.

23. The method of claim 22, wherein:
(a) the consecutive dose is administered at a time when the pancreatitis, renal insufficiency, and/or gastrointestinal inflammation has been treated; and/or
(b) the consecutive dose is administered at a time when the pancreatitis, renal insufficiency, and/or gastrointestinal inflammation has subsided.

24. The method of claim 22, further comprising administering to the subject a lymphodepleting chemotherapy.

25. The method of claim 24, wherein the lymphodepleting chemotherapy:
(a) comprises a therapeutically effective amount of cyclophosphamide and/or fludarabine;
(b) comprises a therapeutically effective amount of cyclophosphamide and fludarabine; and/or
(c) comprises a therapeutically effective amount of cyclophosphamide, wherein the therapeutically effective amount of cyclophosphamide is 300 mg/m²/day; and/or
(d) comprises a therapeutically effective amount of fludarabine, wherein the therapeutically effective amount of fludarabine is 30 mg/m²/day; and/or
(e) is administered to the subject prior to administering the first dose of cells; and/or
(f) is administered to the subject four to six days prior to administering the first dose of cells; and/or
(g) is administered to the subject consecutively for three days.
